(19) United States
(12) Patent Application Publication
(10) Pub. No.: US 2024/0369564 A1
(43) Pub. Date: Nov. 7, 2024

(54) METHODS OF DISEASE DIAGNOSTICS UTILIZING MICROBIAL EXTRACELLULAR VESICLE (MEV) ANALYTES

(71) Applicant: Micronoma, Inc., San Diego, CA (US)

(72) Inventors: Serena Fraraccio, San Diego, CA (US); Eddie Adams, San Diego, CA (US); Stephen Wandro, San Diego, CA (US)

(21) Appl. No.: 18/580,801

(22) PCT Filed: Jul. 19, 2022
(86) PCT No.: PCT/US2022/037647

§ 371 Date: Jan. 19, 2024

Related U.S. Application Data
(60) Provisional application No. 63/223,725, filed on Jul. 20, 2021.

(51) Int. Cl. G01N 33/574; G01N 33/569; G16H 10/40; G16H 50/20

(52) U.S. Cl. CPC G01N 33/57488; G01N 33/569; G16H 10/40; G16H 50/20; G01N 2800/52

(57) ABSTRACT

Methods and systems are presented herein for predicting a disease of a subject through a combination of fungal and non-fungal molecular analyte features of a biological sample.

### CROSS-REFERENCE

This application claims the benefit of U.S. Provisional Application No. 63/223,725 filed Jul. 20, 2021, which application is incorporated herein by reference.

### SUMMARY

The disclosure of the present invention provides an affinity-based method of isolating microbial extracellular vesicles (MEVs) for molecular analysis and using the results of said molecular analysis to form a diagnosis of a subject's health or disease status. Specifically, the present invention provides methods for affinity-based isolation of MEVs and non-microbial extracellular vesicles (non-microbial EVs) present in a liquid biopsy sample and methods for using the presence or abundance of vesicle-associated analytes (VAA) to diagnose and classify disease in a subject. In some cases, the subject may be a mammal or a non-mammal.

The methods of the present invention disclosed herein generate a diagnostic method capable of diagnosing and classifying disease based on the disease-specific presence and or abundance of VAA. In some embodiments, characterization of MEVs present in a liquid biological sample (e.g., liquid biopsy sample) may enable diagnosis of conditions that may otherwise be intractable to non-invasive or minimally invasive procedures. For example, 16S rDNA sequencing analysis of blood-derived MEVs in an Alzheimer disease mouse model revealed significant taxonomic differences between the Alzheimer's mice and wild-type controls (PMID: 29302204), suggesting that MEV-derived DNA can provide disease-specific microbial signatures for a pathology that is traditionally only fully diagnosed via post-mortem analysis of brain tissue. Similarly, MEVs isolated from urine samples have facilitated bacterial metagenomic analyses of individuals with autism-spectrum disorder (PMID: 29093639).

Aspects disclosed herein provide a method of diagnosing a disease in a subject based on disease-associated MEVs in a liquid biological sample comprising: (a) enriching the MEVs with one or more affinity capture reagents; and (b) detecting the disease-associated abundance of molecular analytes from the MEVs. In some embodiments, (a) may result in the separation of MEVs from non-microbial EVs. In some embodiments, (b) may further involve quantifying the disease-associated abundance of molecular analytes. In some embodiments, the MEVs may comprise vesicle-associated cell-free microbial DNA, microbial RNA, non-microbial DNA, non-microbial RNA, microbial proteins, non-microbial proteins, microbial metabolites, or non-microbial metabolites, microbial lipids, non-microbial lipids, microbial glycans, non-microbial glycans or any combinations thereof.

In some embodiments, the liquid biological sample may be a liquid biopsy sample. In some embodiments the liquid biopsy sample may comprise plasma, serum, whole blood, urine, cerebral spinal fluid, saliva, lymphatic fluid, sweat, tears, exhaled breath condensate or any dilution, or processed fraction thereof. In some embodiments, the affinity-based enrichment (a) comprises concentrating microbial or non-microbial cell wall molecular motifs. In some embodiments, the microbial or non-microbial cell wall molecular motifs may comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof. In some embodiments, one or more affinity capture reagents may comprise recombinant innate immunity pattern recognition receptors or polyclonal antibodies, monoclonal antibodies, recombinant antibodies, aptamers, single-chain variable fragment (scFv), single-chain antibodies or any combination thereof. In some embodiments, the recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Dectin-1, Dectin-2, Mannose Receptor (CD206), DC-SIGN, SIGNR1, Langerin, mannose binding lectin, ficolin-1, ficolin-2, ficolin-3 or any derivatives thereof. In some embodiments, the derivatives thereof may comprise an epitope tag and the full-length recombinant innate immunity pattern recognition receptors or recombinant soluble ectodomains thereof. In some embodiments, the epitope tag comprises a N- or C-terminal 6×-histidine tag, green fluorescent protein (GFP), myc, hemagglutinin (HA), Fc fusion, biotin, or any combination thereof. In some embodiments, the antibodies, aptamers, single-chain variable fragments (scFv) or single-chain antibodies may contain an epitope tag. In some embodiments, the epitope tag may comprise a N- or C-terminal 6×-histidine tag, green fluorescent protein (GFP), myc, hemagglutinin (HA), Fc fusion, biotin, or any combination thereof. In some embodiments, the affinity reagents may comprise a region to interact with the microbial or non-microbial cell wall molecular motifs. In some cases, this region may bind the microbial or non-microbial cell wall molecular motifs. In some embodiments, the affinity-based enrichment (a) may comprise contacting the liquid biopsy sample with a support comprising covalently immobilized affinity agents. In some cases, the support may comprise a plurality of covalently immobilized affinity agents. In some embodiments, covalently immobilized affinity agents may comprise a region that may interact with the microbial or non-microbial cell wall molecular motifs. In some embodiments, supports may comprise a magnetic bead, an agarose bead, non-magnetic latex, functionalized Sepharose, pH-sensitive polymers or any combinations thereof.

In some embodiments, MEV enrichment from a liquid biological sample may comprise (a.) contacting said liquid biological sample with one or more affinity capture reagents to form capture reagent-molecular motif interaction complexes; (b.) contacting the capture reagent-molecular motif interaction complexes with a support; and (c.) separating the support from the liquid biological sample to concentrate capture reagent-molecular motif interaction complexes. In some embodiments, the one or more affinity capture reagents may comprise an epitope tag. In some embodiments, the support may comprise an epitope tag recognition surface. In some embodiments, the epitope tag recognition surface of the support may interact with the epitope tag of one or more affinity capture reagents. In some embodiments, the epitope tag recognition surface may comprise streptavidin or antibodies specific for 6×-histidine tag, green fluorescent protein (GFP), myc, hemagglutinin (HA), biotin, or any combination thereof. In some embodiments, the epitope tag recognition surface may comprise an anti-species antibody.

In some embodiments, detecting the disease-associated abundance of molecular analytes of the MEVs may comprise nucleic acid sequencing, polymerase chain reaction (PCR)-based, or hybridization-based analysis for nucleic acid analytes. In some embodiments, the disease-associated abundance of molecular analytes may further be quantified. In some cases, the molecular analytes may be on the MEVs or may be within the MEVs. In some embodiments, detecting and/or quantifying molecular analytes may comprise performing mass spectrometry analyses, liquid chromatography-mass spectrometry (LC-MS), high-performance liquid chromatography (HPLC), or any combination thereof. In some embodiments, detecting and/or quantifying molecular analytes may comprise performing immunoassay analysis, such as enzyme-linked immunosorbent assay (ELISA), radioimmunoassay (RIA), fluoroimmunoassay (FIA), chemiluminescent immunoassay (CIA), real-time immunoquantitative PCR (iqPCR), or any combination thereof.

Aspects disclosed herein provide a method of creating a diagnostic for detecting disease in a subject based on disease-associated MEVs in a liquid biological sample comprising: (a) providing a liquid biological sample comprising microbial extracellular vesicles and non-microbial extracellular vesicles; (b) enriching said microbial extracellular vesicles by one or more first affinity capture reagents; (c) enriching said non-microbial extracellular vesicles by one or more second affinity capture reagents; (d) detecting disease-associated abundance of molecular analytes from the microbial extracellular vesicles; (e) detecting disease-associated abundance of molecular analytes from the non-microbial extracellular vesicles; and (f) identifying the disease state in the subject by combining the first and second disease-associated abundances of molecular analytes from the microbial extracellular vesicles and the non-microbial extracellular vesicles to form a signature of disease state. In some embodiments, the microbial extracellular vesicles may comprise vesicle-associated cell-free microbial DNA, microbial RNA, non-microbial DNA, non-microbial RNA, microbial proteins, non-microbial proteins, microbial metabolites, non-microbial metabolites, microbial lipids, non-microbial lipids, microbial glycans, non-microbial glycans or any combination thereof. In some embodiments, the liquid biological sample may comprise plasma, serum, whole blood, urine, cerebral spinal fluid, saliva, lymphatic fluid, sweat, tears, exhaled breath condensate or any dilution, or processed fraction thereof. In some embodiments, the affinity-based enrichment (b) and (c) may comprise concentrating microbial or non-microbial cell wall molecular motifs. In some embodiments, microbial or non-microbial cell wall molecular motifs may comprise canonical cell wall components, such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combination thereof. In some embodiments, the first and second one or more affinity capture reagents may comprise recombinant innate immunity pattern recognition receptors or polyclonal antibodies, monoclonal antibodies, recombinant antibodies, aptamers, single-chain variable fragment (scFv), single-chain antibodies or any combination thereof. In some embodiments, the recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3 or any derivatives thereof. In some embodiments, the recombinant innate immunity pattern recognition receptor derivatives may comprise an epitope tag and the full-length recombinant innate immunity pattern recognition receptors or recombinant soluble ectodomains thereof. In some embodiments, the epitope tag may comprise a N- or C-terminal 6×-histidine tag, green fluorescent protein (GFP), myc, hemagglutinin (HA), Fc fusion, biotin, or any combination thereof. In some embodiments, the antibodies, aptamers, single-chain variable fragments (scFv) and single-chain antibodies may contain an epitope tag. In some embodiments, the epitope tag may comprise a N- or C-terminal 6×-histidine tag, green fluorescent protein (GFP), myc, hemagglutinin (HA), Fc fusion, biotin, or any combination thereof. In some embodiments, the first and second one or more affinity reagents may comprise a region to interact with the microbial or non-microbial cell wall molecular motifs. In some cases, the region may bind to the microbial or non-microbial cell wall molecular motifs. In some embodiments, microbial extracellular vesicle enrichment may comprise incubating said liquid biopsy sample with a support comprising covalently immobilized affinity agents. In some cases, the support may comprise a plurality of covalently immobilized affinity agents. In some embodiments, covalently immobilized affinity agents may comprise a region that interacts with the microbial or non-microbial cell wall molecular motifs. In some cases, the region may bind to the microbial or non-microbial cell wall molecular motifs. In some embodiments, the supports comprise a magnetic bead, an agarose bead, non-magnetic latex, functionalized Sepharose, pH-sensitive polymers or any combinations thereof. In some embodiments, the non-microbial extracellular vesicle affinity capture reagents may comprise polyclonal, monoclonal, recombinant antibodies, aptamers, single-chain variable fragment (scFv), single-chain antibodies or any combination thereof. In some embodiments, the first and second one or more affinity capture reagents may be specific to mammalian antigens CD9, CD63, CD81, glypican 1 (GPC1), Mart-1, TYRP2, Human epidermal growth factor receptors (HER) family members, and EpCAM. In some embodiments, non-microbial extracellular vesicle enrichment may comprise incubating said liquid biological sample with a support comprising covalently immobilized affinity agents. In some embodiments, covalently immobilized affinity agents may comprise a region that may interact with the non-microbial molecular motifs. In some cases, the region may bind to the non-microbial molecular motifs. In some embodiments, the supports may comprise a magnetic bead, an agarose bead, non-magnetic latex, functionalized Sepharose, pH-sensitive polymers or any combinations thereof. In some embodiments, detecting molecular analytes may comprise nucleic acid sequencing or polymerase chain reaction (PCR) analysis for nucleic acid analytes. In some cases, the molecular analytes may further be quantified. In some embodiments, detecting and/or quantifying molecular analytes may comprises performing immunoassay analysis. In some cases, the immunoassay analysis may be, by way of non-limiting examples, enzyme-linked immunosorbent assay (ELISA), radioimmunoassay (RIA), fluoroimmunoassay (FIA), chemiluminescent immunoassay (CIA), real-time immunoquantitative PCR (iqPCR), or any combination thereof.

Aspects disclosed herein provide a method of identifying a disease of a subject, comprising: providing a biological sample from a subject comprising one or more microbial extracellular vesicles; enriching said one or more microbial extracellular vesicles with one or more affinity reagents; detecting one or more molecular analytes from said one or more microbial extracellular vesicles; and identifying said disease of said subject based on said one or more molecular analytes from said one or more microbial extracellular vesicles. In some embodiments, the biological sample comprises non-microbial extracellular vesicles. In some embodiments, the method further comprises quantifying an abundance of said one or more molecular analytes. In some embodiments, the one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. In some embodiments, the one or more molecular analytes of said one or more non-microbial extracellular comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. In some embodiments, the biological sample comprises a liquid biological sample, tissue biological sample, or any combination thereof. In some embodiments, the liquid biological sample comprises plasma, serum, whole blood, urine, cerebral spinal fluid, saliva, sweat, tears, lymphatic fluid, exhaled breath condensate or any dilution, or processed fraction thereof. In some embodiments, the whole blood comprises plasma, white blood cells, red blood cells, platelets, or any combination thereof. In some embodiments, the tissue biological sample comprises tissue homogenate. In some embodiments, the one or more affinity reagents are configured to couple to one or more microbial or one or more non-microbial cell wall molecular motifs. In some embodiments, the one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof. In some embodiments, the one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. In some embodiments, the recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. In some embodiments, the derivatives thereof comprise an epitope tag and full-length recombinant innate immunity pattern recognition receptors or recombinant soluble ectodomains thereof. In some embodiments, the epitope tag comprises a N- or C-terminal 6×-histidine tag, green fluorescent protein (GFP), myc, hemagglutinin (HA), Fc fusion, biotin or any combination thereof. In some embodiments, the one or more antibodies, aptamers, single-chain variable fragments (scFv), or said single-chain antibodies comprise an epitope tag. In some embodiments, the epitope tag comprises a N- or C-terminal 6×-histidine tag, green fluorescent protein (GFP), myc, hemagglutinin (HA), Fc fusion, biotin or any combination thereof. In some embodiments, the one or more affinity reagents comprise a region to interact with said one or more microbial cell wall molecular motifs or said one or more non-microbial cell wall molecular motifs. In some embodiments, the enriching comprises contacting said biological sample with a support comprising covalently immobilized affinity agents. In some embodiments, the covalently immobilized affinity agents comprise a region that interacts with said one or more microbial cell wall molecular motifs or said one or more non-microbial cell wall molecular motifs. In some embodiments, the supports comprise a magnetic bead, an agarose bead, non-magnetic latex, functionalized Sepharose, pH-sensitive polymers or any combination thereof. In some embodiments, enriching comprises: contacting said biological sample with said one or more affinity reagents to form capture reagent-molecular motif interaction complexes; contacting said capture reagent-molecular motif interaction complexes with a support; and separating said support from said biological sample to concentrate said capture reagent-molecular motif interaction complexes. In some embodiments, the one or more affinity reagents comprise an epitope tag. In some embodiments, the support comprises an epitope tag recognition surface. In some embodiments, the epitope tag recognition surface comprises streptavidin, antibodies specific for 6×-histidine tag, green fluorescent protein (GFP), myc, hemagglutinin (HA), biotin, or any combination thereof. In some embodiments, the epitope tag recognition surface comprises an anti-species antibody. In some embodiments, detecting comprises nucleic acid sequencing, polymerase chain reaction (PCR), or hybridization-based analysis of the one or more molecular analytes. In some embodiments, the nucleic acid sequencing comprises whole genome sequencing, shotgun sequencing, next generations sequencing, targeted sequencing, RNA sequencing, methylation sequencing, or any combination thereof. In some embodiments, the detecting comprises performing mass spectrometry analyses, liquid chromatography-mass spectrometry (LC-MS), or high-performance liquid chromatography (HPLC). In some embodiments, the detecting comprises performing immunoassay analysis. In some embodiments, the disease comprises cancer. In some embodiments, the cancer comprises a stage I or stage II cancer. In some embodiments, the cancer comprises bone, breast, lung, colon, brain, skin. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, identifying said cancer comprises predicting said cancer by a predictive model, wherein said predictive model is trained with one or more subjects' one or more molecular analytes obtained from one or more microbial extracellular vesicles and an associated disease of said one more subjects. In some embodiments, the predictive model is configured to receive said one or more molecular analytes of said subject as an input, and output said disease of said subject. In some embodiments, the predictive model is configured to predict one or more cancers, one or more subtypes of cancer, the anatomical locations of one or more cancers, or any combination thereof of said subject. In some embodiments, the predictive model is configured to predict a stage of said cancer, prognosis of said cancer, mutation status of said cancer, future immunotherapy response of said cancer, an optimal therapy to treat said cancer, or any combination thereof. In some embodiments, the predictive model is configured to predict said cancer among one or more cancer types of said subject to identify a specific cancer type of said one or more cancer types. In some embodiments, the predictive model comprises a machine learning model, wherein said machine learning model comprises a regularized machine learning model, ensemble of machine learning models, or any combination thereof. In some embodiments, the predictive model comprises a random forest, neural network, naïve bayes, support vector machines, learning regression, k-nearest neighbors, k-means, decision tree, logistic regression, gradient boosting, or any combination thereof. In some embodiments, enriching said microbial extracellular vesicles improves an accuracy of said predictive model by at least 1%, at least 5%, at least 10%, at least 15%, or at least 20% when predicting said cancer of said subject. In some embodiments, the subject comprises a non-human mammal or a human subject.

Aspects of the disclosure provide a method of identifying a disease of a subject comprising: providing biological sample of a subject comprising one or more microbial extracellular vesicles and one or more non-microbial extracellular vesicles; enriching said one or more microbial extracellular vesicles with one or more first affinity reagents and said one or more non-microbial extracellular vesicles with one or more second affinity reagents, thereby generating one or more enriched microbial extracellular vesicles and one or more enriched non-microbial extracellular vesicles; detecting a first abundance of one or more molecular analytes from said enriched one or more microbial extracellular vesicles and a second abundance of one or more molecular analytes from said enriched one or more non-microbial extracellular vesicles; and identifying said disease of said subject from an association between a combination of said first abundance of one or more molecular analytes and said second abundances of one or more molecular analytes, and a model of diseases associated with a third abundance of one or more molecular analytes from one or more microbial extracellular vesicles and a fourth abundance of one or more molecular analytes from one or more non-microbial extracellular vesicles. In some embodiments, detecting comprises quantifying said first abundance of one or more molecular analytes and said second abundance of one or more molecular analytes. In some embodiments, the one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. In some embodiments, the one or more molecular analytes of said one or more non-microbial extracellular vesicles comprises cell-free non-microbial DNA, cell-free non-microbial RNA, non-microbial DNA, non-microbial RNA, non-microbial proteins, non-microbial metabolites, non-microbial lipids, non-microbial glycans, or any combination thereof. In some embodiments, the biological sample comprises a liquid biological sample, tissue biological sample, or any combination thereof. In some embodiments, the liquid biological sample comprises plasma, serum, whole blood, urine, cerebral spinal fluid, saliva, sweat, tears, lymphatic fluid, exhaled breath condensate, or any combination, any dilution, or processed fraction thereof. In some embodiments, the whole blood comprises white blood cells, plasma, red blood cells, platelets, or any combination thereof. In some embodiments, the tissue biological sample comprises tissue homogenate. In some embodiments, enriching comprises concentrating one or more microbial cell wall molecular motifs or one or more non-microbial cell wall molecular motifs. In some embodiments, the one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof. In some embodiments, the one or more first affinity reagents or said one or more second affinity reagents comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof. In some embodiments, the recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof. In some embodiments, the derivatives thereof comprise an epitope tag, full-length recombinant innate immunity pattern recognition receptors, or recombinant soluble ectodomains thereof. In some embodiments, the one or more antibodies, said aptamers, said single-chain variable fragments (scFv), and said single-chain antibodies comprise an epitope tag. In some embodiments, the epitope tag comprises a N- or C-terminal 6×-histidine tag, green fluorescent protein (GFP), myc, hemagglutinin (HA), Fc fusion, biotin or any combination thereof. In some embodiments, the one or more first affinity reagents and said one or more second affinity reagents comprise a region to interact with said one or more microbial cell wall molecular motifs or said one or more non-microbial cell wall molecular motifs. In some embodiments, enriching comprises incubating said biological sample with a support comprising covalently immobilized affinity agents. In some embodiments, the covalently immobilized affinity agents comprise a region that interacts with said one or more microbial cell wall molecular motifs or said one or more non-microbial cell wall molecular motifs. In some embodiments, the supports comprise a magnetic bead, an agarose bead, non-magnetic latex, functionalized Sepharose, pH-sensitive polymers or any combinations thereof. In some embodiments, the one or more first affinity reagents or said one or more second affinity reagents are specific to mammalian antigens CD9, CD63, CD81, glypican 1 (GPC1), Mart-1, TYRP2, Human epidermal growth factor receptors (HER) family members, EpCAM, or any combination thereof. In some embodiments, detecting comprises performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with said one or more molecular analytes of said one or more microbial extracellular vesicles or said one or more molecular analytes from said one or more non-microbial extracellular vesicles. In some embodiments, the nucleic acid sequencing comprises whole genome sequencing, shotgun sequencing, next generations sequencing, targeted sequencing, RNA sequencing, methylation sequencing, or any combination thereof. In some embodiments, the model comprises a predictive model, wherein the predictive model is trained with one or more subjects' said third abundance of one or more molecular analytes from said one or more microbial extracellular vesicles, said fourth abundance of one or more molecular analytes from said one or more non-microbial extracellular vesicles, and a corresponding disease of said one or more subjects. In some embodiments, the predictive model is configured to receive said first abundance of one or more molecular analytes and said second abundance of one or more molecular analytes as an input and output a prediction of said disease of said subject. In some embodiments, the said disease comprises cancer. In some embodiments, the cancer comprises a stage I or stage II cancer. In some embodiments, the cancer comprises bone, breast, lung, colon, brain, skin, ovary, pancreas, or any combination thereof type of cancer. In some embodiments, the predictive model is configured to predict one or more cancers, one or more subtypes of cancer, anatomical locations of one or more cancers, or any combination thereof in said subject. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, the predictive model comprises a machine learning model. In some embodiments, the machine learning model comprises one or more machine learning models, a regularized machine learning model, an ensemble of machine learning models, or any combination thereof. In some embodiments, the predictive model comprises a random forest, neural network, naïve bayes, support vector machines, linear regression, k-nearest neighbors, k-means, decision tree, logistic regression, gradient boosting, or any combination thereof predictive model. In some embodiments, the subject comprises a human or a non-human mammal. In some embodiments, enriching said one or more microbial extracellular vesicles and said one or more non-microbial extracellular vesicles improves an accuracy of said predictive model by at least 1%, at least 5%, at least 10%, at least 15%, or at least 20% when predicting said cancer of said subject. In some embodiments, an area under a receiver operating characteristic curve of said predictive model when predicting said disease of said subject is increase by at least 1%, at least 2%, at least 4%, at least 5%, or at least 10%, when said combination of said first abundance of one or more molecular analytes and said second abundance of one or more molecular analytes are provided as said input to said predictive model.

Aspects of the disclosure provide a method of identifying a treatment for a disease of a subject, comprising: providing a biological sample of a subject comprising one or more microbial extracellular vesicles; enriching said one or more microbial extracellular vesicles with one or more affinity reagents, thereby generating one or more enriched microbial extracellular vesicles; detecting one or more molecular analytes from said one or more enriched microbial extracellular vesicles; and identifying said treatment for said disease of said subject based on said one or more molecular analytes. In some embodiments, the biological sample comprises non-microbial extracellular vesicles and said one or more microbial extracellular vesicles. In some embodiments, the method further comprises quantifying an abundance of said one or more molecular analytes. In some embodiments, the one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. In some embodiments, the one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. In some embodiments, the biological sample comprises a liquid biological sample, tissue biological sample, or any combination thereof. In some embodiments, the liquid biological sample comprises plasma, serum, whole blood, urine, cerebral spinal fluid, saliva, sweat, tears, lymphatic fluid, exhaled breath condensate or any dilution, or processed fraction thereof. In some embodiments, the whole blood comprises plasma, white blood cells, red blood cells, platelets, or any combination thereof. In some embodiments, the tissue biological sample comprises tissue homogenate. In some embodiments, the one or more affinity reagents are configured to couple to one or more microbial or one or more non-microbial cell wall molecular motifs. In some embodiments, the one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof. In some embodiments, the one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. In some embodiments, the recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. In some embodiments, the derivatives thereof comprise an epitope tag and full-length recombinant innate immunity pattern recognition receptors or recombinant soluble ectodomains thereof. In some embodiments, the epitope tag comprises a N- or C-terminal 6×-histidine tag, green fluorescent protein (GFP), myc, hemagglutinin (HA), Fc fusion, biotin, or any combination thereof. In some embodiments, the one or more antibodies, aptamers, single-chain variable fragments (scFv), or said single-chain antibodies comprise an epitope tag. In some embodiments, the epitope tag comprises a N- or C-terminal 6×-histidine tag, green fluorescent protein (GFP), myc, hemagglutinin (HA), Fc fusion, biotin or any combination thereof. In some embodiments, the one or more affinity reagents comprise a region to interact with said one or more microbial cell wall molecular motifs or said one or more non-microbial cell wall molecular motifs. In some embodiments, enriching comprises contacting said biological sample with a support comprising covalently immobilized affinity agents. In some embodiments, the covalently immobilized affinity agents comprise a region that interacts with said one or more microbial cell wall molecular motifs or said one or more non-microbial cell wall molecular motifs. In some embodiments, the supports comprise a magnetic bead, an agarose bead, non-magnetic latex, functionalized Sepharose, pH-sensitive polymers or any combinations thereof. In some embodiments, enriching comprises: contacting said biological sample with said one or more affinity reagents to form capture reagent-molecular motif interaction complexes; contacting said capture reagent-molecular motif interaction complexes with a support; and separating said support from said biological sample to concentrate said capture reagent-molecular motif interaction complexes. In some embodiments, the one or more affinity reagents comprise an epitope tag. In some embodiments, the support comprises an epitope tag recognition surface. In some embodiments, the epitope tag recognition surface comprises streptavidin, antibodies specific for 6×-histidine tag, green fluorescent protein (GFP), myc, hemagglutinin (HA), biotin, or any combination thereof. In some embodiments, the epitope tag recognition surface comprises an anti-species antibody. In some embodiments, detecting comprises nucleic acid sequencing, polymerase chain reaction (PCR)-based, or hybridization-based analysis for nucleic acid analytes. In some embodiments, the nucleic acid sequencing comprises whole genome sequencing, shotgun sequencing, next generations sequencing, targeted sequencing, RNA sequencing, methylation sequencing, or any combination thereof. In some embodiments, detecting comprises performing mass spectrometry analyses, liquid chromatography-mass spectrometry (LC-MS), or high-performance liquid chromatography (HPLC). In some embodiments, detecting comprises performing immunoassay analysis. In some embodiments, the disease comprises cancer. In some embodiments, the cancer comprises a stage I or stage II cancer. In some embodiments, the cancer comprises bone, breast, lung, colon, brain, skin, ovary, pancreas, or any combination thereof type of cancer. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, identifying said treatment for said disease comprises predicting said treatment by a predictive model, wherein said predictive model is trained with one or more subjects' one or more molecular analytes obtained from one or more microbial extracellular vesicles and an associated treatment for said disease of said one more subjects. In some embodiments, the predictive model is configured to receive said one or more molecular analytes of said subject an input, and output said treatment for said disease of said subject. In some embodiments, the predictive model comprises a machine learning model, wherein said machine learning model comprises a regularized machine learning model, ensemble of machine learning models, or any combination thereof. In some embodiments, the predictive model comprises a random forest, neural network, naïve bayes, support vector machines, learning regression, k-nearest neighbors, k-means, decision tree, logistic regression, gradient boosting, or any combination thereof. In some embodiments, enriching said one or more microbial extracellular vesicles improves an accuracy of said predictive model by at least 1%, at least 5%, at least 10%, at least 15%, or at least 20% when predicting said treatment of said subject. In some embodiments, the subject comprises a non-human mammal or a human subject. In some embodiments, the treatment comprises a repurposed treatment, which may or may not have been originally approved for targeting cancer. In some embodiments, the treatment comprises a small molecule, a biologic, a probiotic, a virus, a bacteriophage, an immunotherapy, a broad-spectrum antibiotic, or any combination thereof. In some embodiments, the probiotic comprises an engineered bacterium strain or ensemble of engineered bacteria. In some embodiments, the treatment comprises an adjuvant given in combination with a primary treatment against said cancer to improve an efficacy of said primary treatment. In some embodiments, the treatment comprises adoptive cell transfer to target microbial antigens associated with said cancer or cancer microenvironment. In some embodiments, the treatment comprises a cancer vaccine that exploits microbial antigens associated with said cancer or cancer microenvironment. In some embodiments, the treatment comprises a monoclonal antibody against microbial antigens associated with said cancer or cancer microenvironment. In some embodiments, the treatment comprises an antibody-drug conjugate designed to at least partially target microbial antigens associated with said cancer or cancer microenvironment. In some embodiments, the treatment comprises a multi-valent antibody, antibody fragment, or antibody derivative thereof designed to at least partially target one or more microbial antigens associated with said cancer or cancer microenvironment. In some embodiments, the treatment comprises a targeted antibiotic against a particular kind of microbe or class of functionally or biologically similar microbes. In some embodiments, two or more of the following treatment types are combined such that at least one type exploits said cancer microbial presence or abundance to enhance overall therapeutic efficacy: small molecules, biologics, engineered host-derived cell types, probiotics, engineered bacteria, natural-but-selective viruses, engineered viruses, and bacteriophages.

Aspects of the disclosure provide a method of identifying a treatment for a disease of a subject, comprising: providing a biological sample of a subject comprising one or more microbial extracellular vesicles and one or more non-microbial extracellular vesicles; enriching said one or more microbial extracellular vesicles with one or more first affinity reagents and said one or more non-microbial extracellular vesicles with one or more second affinity reagents, thereby generating one or more enriched microbial extracellular vesicles and one or more enriched non-microbial extracellular vesicles; detecting a first abundance of one or more molecular analytes from said enriched one or more microbial extracellular vesicles and a second abundance of one or more molecular analytes from said enriched one or more non-microbial extracellular vesicles; and identifying said treatment for said disease of said subject from an association between a combination of said first abundance of one or more molecular analytes and said second abundance of one or more molecular analytes, and a model of disease treatments associated with a third abundance of one or more molecular analytes from one or more microbial extracellular vesicles and a fourth abundance of one or more molecular analytes from one or more non-microbial extracellular vesicles. In some embodiments, detecting comprises quantifying said first abundance of one or more molecular analytes and said second abundance of one or more molecular analytes. In some embodiments, the one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. In some embodiments, the one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. In some embodiments, the biological sample comprises a liquid biological sample, a tissue biological sample, or any combination thereof. In some embodiments, the liquid biological sample comprises plasma, serum, whole blood, urine, cerebral spinal fluid, saliva, sweat, tears, lymphatic fluid, exhaled breath condensate, or any combination, any dilution, or processed fraction thereof. In some embodiments, the whole blood comprises white blood cells, plasma, red blood cells, platelets, or any combination thereof. In some embodiments, the tissue biological sample comprises tissue homogenate. In some embodiments, enriching comprises concentrating one or more microbial or one or more non-microbial cell wall molecular motifs. In some embodiments, the one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof. In some embodiments, the one or more first affinity reagents or said one or more second affinity reagents comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof. In some embodiments, the recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof. In some embodiments, the derivatives thereof comprise an epitope tag, full-length recombinant innate immunity pattern recognition receptors, or recombinant soluble ectodomains thereof. In some embodiments, the one or more antibodies, said aptamers, said single-chain variable fragments (scFv), and said single-chain antibodies comprise an epitope tag. In some embodiments, the epitope tag comprises a N- or C-terminal 6×-histidine tag, green fluorescent protein (GFP), myc, hemagglutinin (HA), Fc fusion, biotin, or any combination thereof. In some embodiments, the one or more first affinity reagents and said one or more second affinity reagents comprise a region to interact with said one or more microbial cell wall molecular motifs or said one or more non-microbial cell wall molecular motifs. In some embodiments, enriching comprises incubating said liquid biological sample with a support comprising covalently immobilized affinity agents. In some embodiments, the covalently immobilized affinity agents comprise a region that interacts with said one or more microbial cell wall molecular motifs or said one or more non-microbial cell wall molecular motifs. In some embodiments, the supports comprise a magnetic bead, an agarose bead, non-magnetic latex, functionalized Sepharose, pH-sensitive polymers or any combinations thereof. In some embodiments, the one or more second affinity reagents comprise polyclonal, monoclonal, recombinant antibodies, aptamers, single-chain variable fragment (scFv), single-chain antibodies, or any combination thereof. In some embodiments, the one or more first affinity reagents or said one or more second affinity reagents are specific to mammalian antigens CD9, CD63, CD81, glypican 1 (GPC1), Mart-1, TYRP2, Human epidermal growth factor receptors (HER) family members, EpCAM, or any combination thereof. In some embodiments, detecting comprises performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with said one or more molecular analytes of said one or more microbial extracellular vesicles or said one or more molecular analytes from said one or more non-microbial extracellular vesicles. In some embodiments, the nucleic acid sequencing comprises whole genome sequencing, shotgun sequencing, next generations sequencing, targeted sequencing, RNA sequencing, methylation sequencing, or any combination thereof. In some embodiments, the model comprises a predictive model, wherein said predictive model is trained with one or more subjects' said third abundance of one or more molecular analytes from said one or more microbial extracellular vesicles, said fourth abundance of one or more molecular analytes from said one or more non-microbial extracellular vesicles, and a corresponding treatment for said disease of said one or more subjects. In some embodiments, the predictive model is configured to receive said first abundance of one or more molecular analytes and said second abundance of one or more molecular analytes as an input and output a prediction of said treatment for said disease of said subject. In some embodiments, the disease comprises cancer. In some embodiments, the cancer comprises a stage I or stage II cancer. In some embodiments, the cancer comprises bone, breast, lung, colon, brain, skin, ovary, pancreas, or any combination thereof type of cancer. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, the predictive model comprises a machine learning model. In some embodiments, the machine learning model comprises one or more machine learning models, a regularized machine learning model, an ensemble of machine learning models, or any combination thereof. In some embodiments, the predictive model comprises a random forest, neural network, naïve bayes, support vector machines, linear regression, k-nearest neighbors, k-means, decision tree, logistic regression, gradient boosting, or any combination thereof predictive model. In some embodiments, the subject comprises a human or a non-human mammal. In some embodiments, enriching said one or more microbial extracellular vesicles and said one or more non-microbial extracellular vesicles improves an accuracy of said predictive model by at least 1%, at least 5%, at least 10%, at least 15%, or at least 20% when predicting said treatment for said cancer of said subject. In some embodiments, an area under a receiver operating characteristic curve of said predictive model predicting said treatment for said disease of said subject is increase by at least 1%, at least 2%, at least 4%, at least 5%, or at least 10%, when said combination of said first abundance of one or more molecular analytes and said second abundance of one or more molecular analytes are provided as said input to said predictive model. In some embodiments, the treatment comprises a repurposed treatment, which may or may not have been originally approved for targeting cancer. In some embodiments, the treatment comprises a small molecule, a biologic, a probiotic, a virus, a bacteriophage, an immunotherapy, a broad-spectrum antibiotic, or any combination thereof. In some embodiments, the probiotic comprises an engineered bacterium strain or ensemble of engineered bacteria. In some embodiments, the treatment comprises an adjuvant given in combination with a primary treatment against said cancer to improve an efficacy of said primary treatment. In some embodiments, the treatment comprises adoptive cell transfer to target microbial antigens associated with said cancer or cancer microenvironment. In some embodiments, the treatment comprises a cancer vaccine that exploits microbial antigens associated with said cancer or cancer microenvironment. In some embodiments, the treatment comprises a monoclonal antibody against microbial antigens associated with said cancer or cancer microenvironment. In some embodiments, the treatment comprises an antibody-drug conjugate designed to at least partially target microbial antigens associated with said cancer or cancer microenvironment. In some embodiments, the treatment comprises a multi-valent antibody, antibody fragment, or antibody derivative thereof designed to at least partially target one or more microbial antigens associated with said cancer or cancer microenvironment. In some embodiments, the treatment comprises a targeted antibiotic against a particular kind of microbe or class of functionally or biologically similar microbes. In some embodiments, two or more of the following treatment types are combined such that at least one type exploits said cancer microbial presence or abundance to enhance overall therapeutic efficacy: small molecules, biologics, engineered host-derived cell types, probiotics, engineered bacteria, natural-but-selective viruses, engineered viruses, and bacteriophages.

Aspects of the disclosure provide a method of training a predictive model, comprising: providing a biological sample of one or more subjects comprising one or more microbial extracellular vesicles, and an associated health classification of said one or more subjects; enriching said one or more microbial extracellular vesicles with one or more affinity reagents, thereby generating one or more enriched microbial extracellular vesicles; detecting one or more molecular analytes from said enriched one or more microbial extracellular vesicles; and training said predictive model with said one or more molecular analytes and said associated health classification of said one or more subjects. In some embodiments, the biological sample comprises non-microbial extracellular vesicles. In some embodiments, the method further comprises quantifying an abundance of said one or more molecular analytes. In some embodiments, the one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. In some embodiments, the one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. In some embodiments, the biological sample comprises a liquid biological sample, tissue biological sample, or any combination thereof. In some embodiments, the liquid biological sample comprises plasma, serum, whole blood, urine, cerebral spinal fluid, saliva, sweat, tears, lymphatic fluid, exhaled breath condensate or any dilution, or processed fraction thereof. In some embodiments, the whole blood comprises plasma, white blood cells, red blood cells, platelets, or any combination thereof. In some embodiments, the tissue biological sample comprises tissue homogenate. In some embodiments, the one or more affinity reagents are configured to couple to one or more microbial cell wall molecular motifs or one or more non-microbial cell wall molecular motifs. In some embodiments, the one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof. In some embodiments, the one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. In some embodiments, the recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. In some embodiments, the derivatives thereof comprise an epitope tag and full-length recombinant innate immunity pattern recognition receptors or recombinant soluble ectodomains thereof. In some embodiments, the epitope tag comprises a N- or C-terminal 6×-histidine tag, green fluorescent protein (GFP), myc, hemagglutinin (HA), Fc fusion, biotin, or any combination thereof.

The method of claim 191, wherein said one or more antibodies, aptamers, single-chain variable fragments (scFv), or said single-chain antibodies comprise an epitope tag. In some embodiments, the epitope tag comprises a N- or C-terminal 6×-histidine tag, green fluorescent protein (GFP), myc, hemagglutinin (HA), Fc fusion, biotin or any combination thereof. In some embodiments, the one or more affinity reagents comprise a region to interact with said one or more microbial cell wall molecular motifs or said one or more non-microbial cell wall molecular motifs. In some embodiments, enriching comprises contacting said liquid biological sample with a support comprising covalently immobilized affinity agents. In some embodiments, the covalently immobilized affinity agents comprise a region that interacts with said one or more microbial cell wall molecular motifs or said one or more non-microbial cell wall molecular motifs. In some embodiments, the supports comprise a magnetic bead, an agarose bead, non-magnetic latex, functionalized Sepharose, pH-sensitive polymers or any combinations thereof. In some embodiments, enriching comprises: contacting said liquid biological sample with said one or more affinity reagents to form capture reagent-molecular motif interaction complexes; contacting said capture reagent-molecular motif interaction complexes with a support; and separating said support from said liquid biological sample to concentrate said capture reagent-molecular motif interaction complexes. In some embodiments, the one or more affinity reagents comprise an epitope tag. In some embodiments, the support comprises an epitope tag recognition surface. In some embodiments, the epitope tag recognition surface comprises streptavidin, antibodies specific for 6×-histidine tag, green fluorescent protein (GFP), myc, hemagglutinin (HA), biotin, or any combination thereof. In some embodiments, the epitope tag recognition surface comprises an anti-species antibody. In some embodiments, detecting comprises nucleic acid sequencing, polymerase chain reaction (PCR)-based, or hybridization-based analysis for nucleic acid analytes. In some embodiments, nucleic acid sequencing comprises whole genome sequencing, shotgun sequencing, next generations sequencing, targeted sequencing, RNA sequencing, methylation sequencing, or any combination thereof. In some embodiments, the detecting comprises performing mass spectrometry analyses, liquid chromatography-mass spectrometry (LC-MS), or high-performance liquid chromatography (HPLC). In some embodiments, the detecting comprises performing immunoassay analysis. In some embodiments, the trained predictive model is configured to receive one or more molecular analytes of a subject as an input and output a predicted disease of said subject, a treatment for said disease of said subject, or any combination thereof. In some embodiments, the disease comprises cancer. In some embodiments, the cancer comprises a stage I or stage II cancer. In some embodiments, the cancer comprises bone, breast, lung, colon, brain, skin, ovary, pancreas, or any combination thereof type of cancer. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, the trained predictive model is configured to predict one or more cancers, one or more subtypes of cancer, the anatomical locations of one or more cancers, or any combination thereof of said subject. In some embodiments, the trained predictive model is configured to predict a stage of said cancer, prognosis of said cancer, mutation status of said cancer, future immunotherapy response of said cancer, an optimal therapy to treat said cancer, or any combination thereof. In some embodiments, the trained predictive model is configured to predict said cancer among one or more cancer types of said subject to identify a specific cancer type of said one or more cancer types. In some embodiments, the trained predictive model comprises a machine learning model, wherein said machine learning model comprises a regularized machine learning model, ensemble of machine learning models, or any combination thereof. In some embodiments, the trained predictive model comprises a random forest, neural network, naïve bayes, support vector machines, learning regression, k-nearest neighbors, k-means, decision tree, logistic regression, gradient boosting, or any combination thereof. In some embodiments, enriching said microbial extracellular vesicles improves an accuracy of said trained predictive model by at least 1%, at least 5%, at least 10%, at least 15%, or at least 20% when predicting said cancer of said subject. In some embodiments, the subject comprises a non-human mammal or a human subject. In some embodiments, the health classification comprises cancerous, non-cancerous disease, or non-cancerous non-disease.

Aspects of the disclosure provide a computer-implemented method of training a predictive model, comprising: receiving one or more subjects' biological sample sequencing data and corresponding health classifications from a database, wherein said biological sample sequencing data comprises sequences of one or more analytes of one or more microbial extracellular vesicles; and training said predictive model with said biological sample sequencing data and said corresponding health classification of said one or more subjects.

A computer system configured to identify a disease of a subject, comprising: one or more processors; and a non-transient computer readable storage medium including software, wherein said software comprises executable instruction that, as a result of execution, cause said one or more processors of said computer system to: receive a biological sample of a subject comprising one or more microbial extracellular vesicles; (ii) enrich said one or more microbial extracellular vesicles with one or more affinity reagents; (iii) detect one or more molecular analytes from said one or more microbial extracellular vesicles; and (iv) identify said disease of said subject based on said one or more molecular analytes obtained from said one or more microbial extracellular vesicles.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example liquid biopsy diagnostic development workflow, as described in embodiments herein.

FIG. 2 shows an example MEV enrichment workflow, as described in embodiments herein.

FIG. 3 shows an exemplary MEV enrichment workflow, as described in embodiments herein.

FIG. 4 shows an exemplary diagnostic workflow, as described in embodiments herein.

FIG. 5 shows an exemplary predictive model training scheme, as described in embodiments herein.

FIG. 6 shows an exemplary trained predictive model scheme for providing a diagnosis or prediction based on one or more feature data of subjects and optionally subjects' clinical metadata, as described in embodiments herein.

FIG. 7 shows a workflow diagram for a method of identifying a disease of a subject with one or more molecular analytes of one or more MEVs, as described in embodiments herein.

FIG. 8 shows a workflow diagram for a method of identifying a disease of a subject with one or more molecular analytes of one or more MEVs and/or non-microbial EVs, as described in embodiments herein.

FIG. 9 shows a workflow diagram for a method of identifying a treatment to treat a disease of a subject with one or more analytes of one or more MEVs, as described in embodiments herein.

FIG. 10 shows a workflow diagram for a method of identifying a treatment to treat a disease of a subject with one or more analytes of one or more MEVs and/or non-microbial EVs, as described in embodiments herein.

FIG. 11 shows a workflow diagram for a method of training a predictive model with one or more analytes of one or more MEVs, as described in embodiments herein.

FIG. 12 shows a workflow diagram for a method of training a predictive model with one or more analytes of one or more MEVs and/or non-microbial EVs, as described in embodiments herein.

FIG. 13 shows a workflow diagram for a method of training a predictive model with one or more subjects' sequencing data of one or more analytes from one or more MEVs and corresponding health classification from a database, as described in embodiments herein.

FIG. 14 shows a workflow diagram for a method of training a predictive model with one or more subjects' sequencing data of one or more analytes from one or more MEVs and/or non-microbial EV's, and corresponding health classification from a database, as described in embodiments herein.

FIG. 15 shows a diagram of a system configured to execute the methods described elsewhere herein, as described in embodiments herein.

FIG. 16 shows a workflow diagram for a method of isolating fraction components of plasma from small cell lung cancer patient, as described in embodiments herein.

FIGS. 17A-17D show graphs of an analysis of the microbiome of the fractions of a plasma from small cell lung cancer patients, as described in embodiments herein.

FIGS. 18A-18C show graphs of an analysis of the microbiome between two independent sets of SCLC patient groups.

### DETAILED DESCRIPTION

The invention provides, in some embodiments, a method to diagnose disease in an animal or human subject using microbial extracellular vesicles from a liquid biological sample (e.g., liquid biopsy sample). This may be accomplished, in some embodiments, by providing a novel, high throughput method of MEV isolation that does not require high speed density gradient centrifugation or size exclusion chromatography, which are low-throughput and time-consuming. Furthermore, high speed density gradient centrifugation and size exclusion chromatography may not be amenable to clinical implementation. In some embodiments, MEV enrichment may be accomplished using agents capable of interacting with molecular features of the MEV. In some cases, the agents may be capable of recognizing or binding with molecular features of the MEV. In some cases, the molecular features of the MEV may be present on the exterior, solvent-accessible, surface of the MEV. Tethering these agents, in some embodiments, to a solid phase, may either directly or indirectly, facilitates retrieval of formed agent-MEV complexes. In some examples, the retrieved agent-MEV complexes may be isolated and may be made available for molecular analysis. Once isolated, the molecular content (e.g., linear nucleic acids, plasmids, circular ribonucleic acids, metabolites, proteins, glycoproteins, lipids, glycolipids, etc.) can be detected and/or quantified. In some embodiments, this detection and/or quantification of the molecular content may be used to establish a diagnostic relationship between the presence and/or abundance of the analyte(s) and a disease. It should be noted that the methods as described herein may be presented in the context of disease diagnostics, but other uses for such methods may be reasonably imaginable and readily implementable to those skilled in the art.

The invention disclosed herein, in some embodiments, may use MEV-derived analytes to diagnose a condition (e.g., cancer). In some embodiments, the disclosed invention may provide better clinical outcomes compared to a pathology report that may include one or more of observed tissue structure, cellular atypia, or other subjective measures, which may be used to diagnose disease. In some embodiments, the disclosed method may focus on the presence of microbial analytes. In some cases, the disclosed method may provide a higher degree of sensitivity by focusing on microbial analytes rather than modified mammalian (e.g., cancer-derived) analytes, which may be present at low frequencies in a background of ‘normal’ (i.e., non-cancerous) mammalian sources. In some embodiments, the methods disclosed herein may achieve such outcomes in liquid biological samples (e.g., liquid biopsy samples), which may require minimal sample preparation and may be minimally invasive.

### Methods

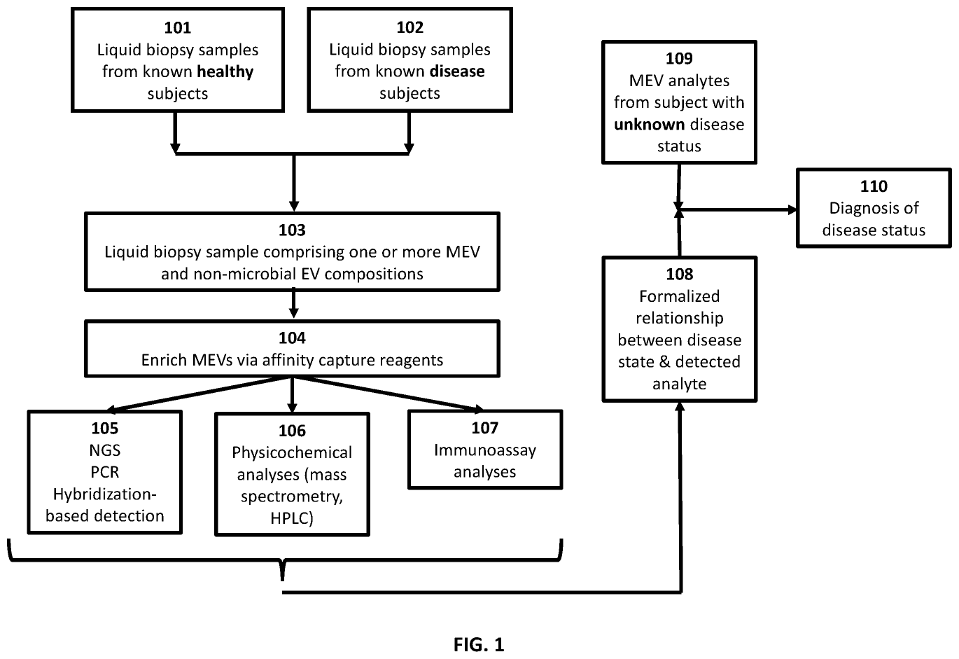

In some embodiments, the disclosure herein provides a disease-specific diagnostic method, as can be seen in FIG. 1, that may comprise: (a) assembling a collection of liquid biopsy samples from known healthy subjects 101 and known disease subjects 102, wherein the liquid biopsy samples may contain a mixture of MEVs and non-microbial EVs 103; (b) performing affinity-based enrichment of MEVs 104 from the collection of liquid biopsy samples; (c) detecting or quantifying MEV-derived analytes 105-107; and (d) formalizing the relationship between the presence and/or abundance of the detected analytes and the disease state being investigated 108. In some embodiments, the formalized relationship may comprise a mathematical representation of an analyte's abundance (e.g., its concentration) that may comprise a relationship with the disease state. In some embodiments, the relationship may be a correlation. In some embodiments, the formalized relationship may comprise a set of features derived from one or more methods of analysis 105-107 that may be computationally ranked. In some cases, the features may be ranked via statistical analysis or statistical learning (e.g., machine learning). In some cases, statistical analysis, or statistical learning (e.g., machine learning) may be used to form a diagnostic feature set indicative of healthy subjects 101 and a diagnostic feature set indicative of disease subjects 102. In some embodiments, MEV analytes from subjects of unknown disease status 109 may be analyzed with respect to the formalized relationship to arrive at a diagnosis of disease state 110.

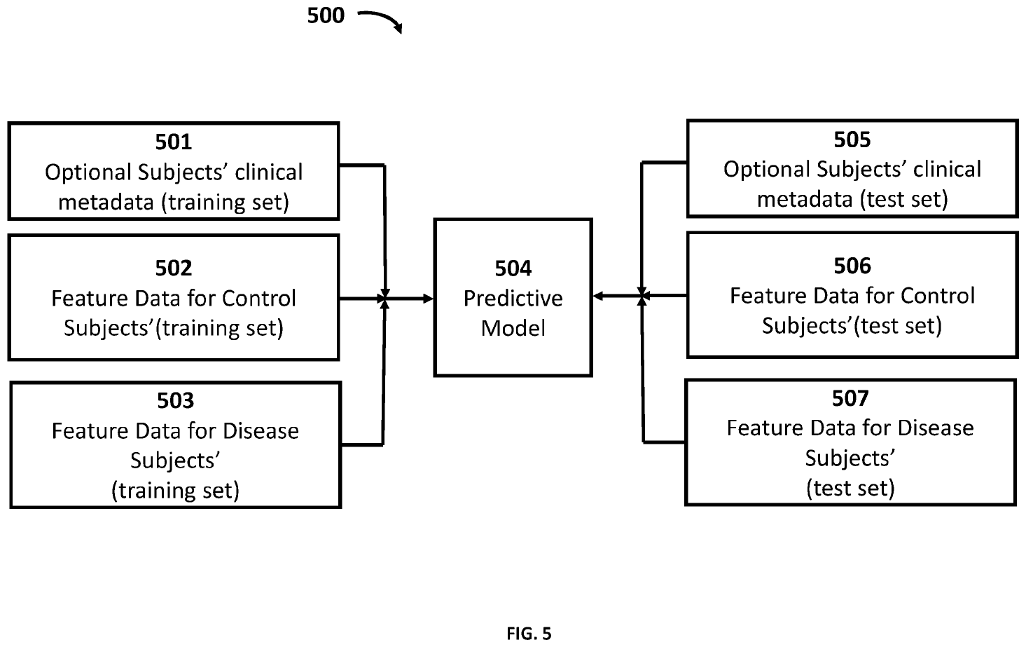

In some cases, the statistical analysis or statistical learning may comprise training a predictive model 500, as seen in FIG. 5. In some cases, the predictive model 504 may comprise one or more machine learning models. In some instances, the one or more machine learning models may comprise a linear regression, logistic regression, decision tree, support vector machine (SVM), naïve bayes, k-nearest neighbors (kNN), k-Means, random forest machine learning model or any combination thereof. In some cases, the machine learning model may be trained using a supervised, unsupervised, or any combination thereof training method. In some instances, the predictive model may be trained to provide a diagnosis or prediction of a disease or condition by one or more features of a liquid biopsy sample, described elsewhere herein. In some cases, the predictive model 504 may be trained with one or more training and test features (501-503, and 505-507) that may comprise MEV-derived nucleic acid analytes data, MEV-derived non-nucleic acid data, or any combination thereof described elsewhere herein. In some cases, the one or more training and test features may comprise training and test features for control (e.g., healthy, disease free, etc.) subjects (502, 506), diseased subjects (503, 507), or any combination thereof of features. In some instances, the training and test features may be labeled by the disease or lack thereof disease state of the subject. In some cases, subjects' clinical metadata (501, 505) may be used as training and test set training data. In some instances, subjects' clinical metadata may comprise subjects' height, age, weight, sex, past medical history, current prescribed medication taken, or any combination thereof. In some cases, the training of the predictive model may comprise supervised or unsupervised training. In some instances, a test and training split ratio of 10:90, 20:80, 30:70, 40:60, 50:50, or any combination thereof training, and test split data may be utilized when training the one or more predictive models. In some instances, the predictive model may be trained with at least one k-fold. In some cases, the predictive model may comprise a previously trained predictive model that may be re-trained (e.g., via transfer learning). In some instances, re-training the predictive model may permit re-training the predictive model with a quantity of data less than what would be required for training an un-trained predictive model.

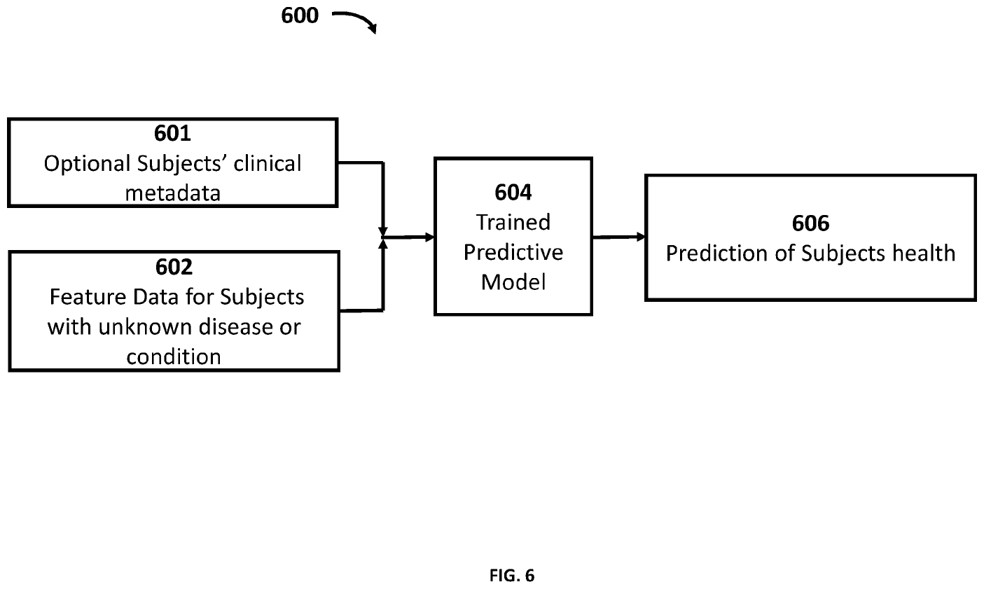

In some embodiments, after the predictive model 504 has been trained, the trained predictive model 604 may predict and/or diagnose whether the one or more subjects possess a presence or lack thereof a disease 600, as can be seen in FIG. 6. In some cases, the input features of the trained predictive model may comprise feature data for subjects with unknown diseases or conditions 602, optionally corresponding subjects' metadata described elsewhere herein 601, or any combination thereof. In some cases, the feature data for subjects with unknown disease or conditions may comprise MEV-derived nucleic acid analytes data, MEV-derived non-nucleic acid data, or any combination thereof, the analysis of which is described elsewhere herein. In some cases, the trained predictive model may process feature data for subjects with unknown diseases or conditions 602, optionally corresponding subjects' metadata 601, and provide a prediction and/or diagnosis 606 of the presence or lack thereof a disease for one or more subjects.

In some embodiments, MEV-derived nucleic acid analytes may be analyzed by sequencing 105, such as next-generation sequencing (NGS), long-read sequencing (e.g., nanopore sequencing), polymerase chain reaction (PCR), nucleic acid hybridization-based methods, or any combination thereof. In some embodiments, MEV-derived non-nucleic acid analytes may be quantified via physicochemical analyses 106 or via immunoassay 107.

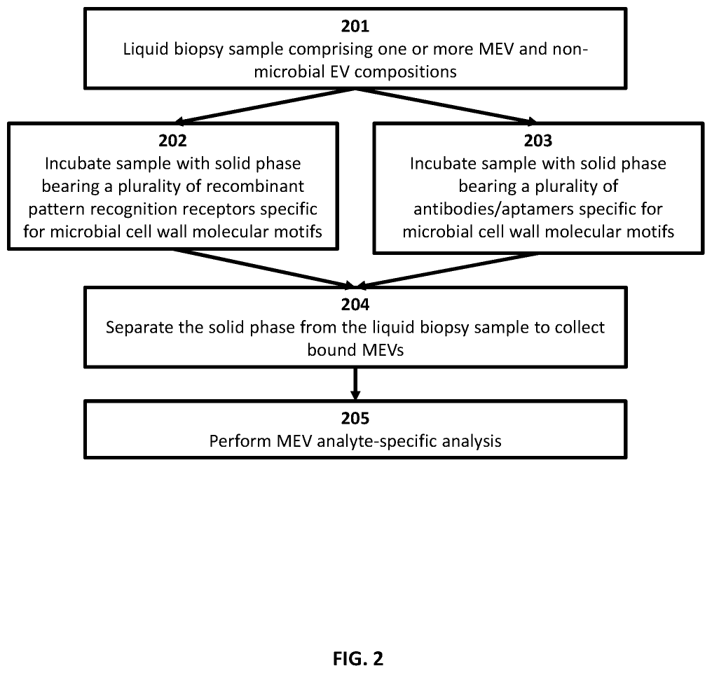

In some embodiments, the affinity-based enrichment of MEVs 104, as shown in FIG. 1, may be performed by incubating a liquid biological sample containing one or more MEV and non-microbial EV compositions 201 with a solid phase bearing one or more MEV capture reagents, as shown in FIG. 2. In some embodiments, a solid phase may be functionalized with recombinant pattern recognition receptors that interact with microbial or non-microbial cell wall molecular motifs 202, which may generate an MEV affinity matrix. In some cases, the recombinant pattern recognition receptors may be immobilized on the solid phase by non-covalent binding interactions, e.g., passive adsorption or via electrostatic interactions, or through covalent binding interactions between solid phase and receptor functional groups. In some embodiments, the solid phase may comprise a mixture of non-covalently immobilized and/or covalently immobilized recombinant pattern recognition receptors. In some embodiments, the solid phase may be functionalized with antibodies and/or aptamers with specificity for microbial or non-microbial cell wall molecular motifs 203, which may generate an MEV affinity matrix. In some embodiments, after incubation of the liquid biological sample 201 with the MEV affinity matrix, the MEV affinity matrix may be separated from the liquid biological sample 204 to collect some or all bound MEVs. In some embodiments, collection of MEV affinity matrix-bound MEVs may be followed by MEV analyte analysis 205 per the methods indicated in FIG. 1, 105-107.

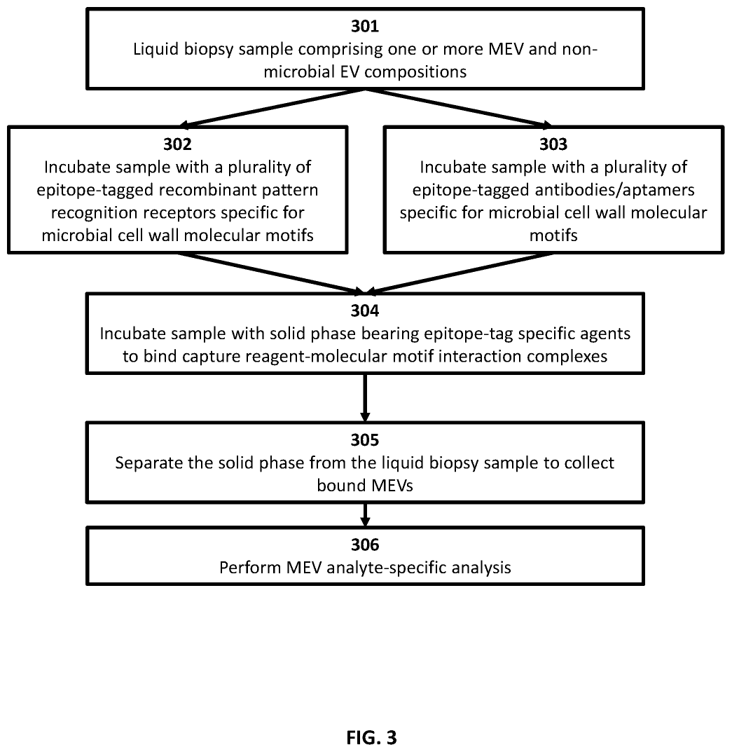

In some embodiments, the affinity-based enrichment of MEVs 104, as shown in FIG. 1 may be performed by incubating a liquid biological sample containing one or more MEV and non-microbial EV compositions 301 with MEV capture reagents, as shown in FIG. 3. In some embodiments, the MEV capture reagents may be solution phase epitope-tags bearing MEV capture reagents. In some embodiments, the epitope-tag bearing MEV capture reagents may comprise recombinant pattern recognition receptors that interact with the microbial or non-microbial cell wall molecular motifs 302. In some cases, the recombinant pattern recognition receptors may be specific to the microbial or non-microbial cell wall molecular motifs 302. In some embodiments, the epitope-tag bearing MEV capture reagents may comprise antibodies and/or aptamers that interact with microbial or non-microbial cell wall molecular motifs 303. In some cases, the antibodies and/or aptamers may have specificity for the microbial or non-microbial cell wall molecular motifs 303. In some embodiments, incubation of the liquid biological sample with one or more epitope-tag bearing MEV capture reagents 302 and/or 303 may be followed by incubation of the sample with a solid phase epitope tag affinity matrix to bind capture reagent-molecular motif interaction complexes 304. In some embodiments, after incubation of the liquid biological sample 304 with the epitope tag affinity matrix, the epitope tag affinity matrix may be separated from the liquid biological sample 305 to collect some or all bound MEVs. In some embodiments, collection of epitope tag affinity matrix-bound MEVs may be followed by MEV analyte analysis 306 per the methods indicated in FIG. 1, 105-107.

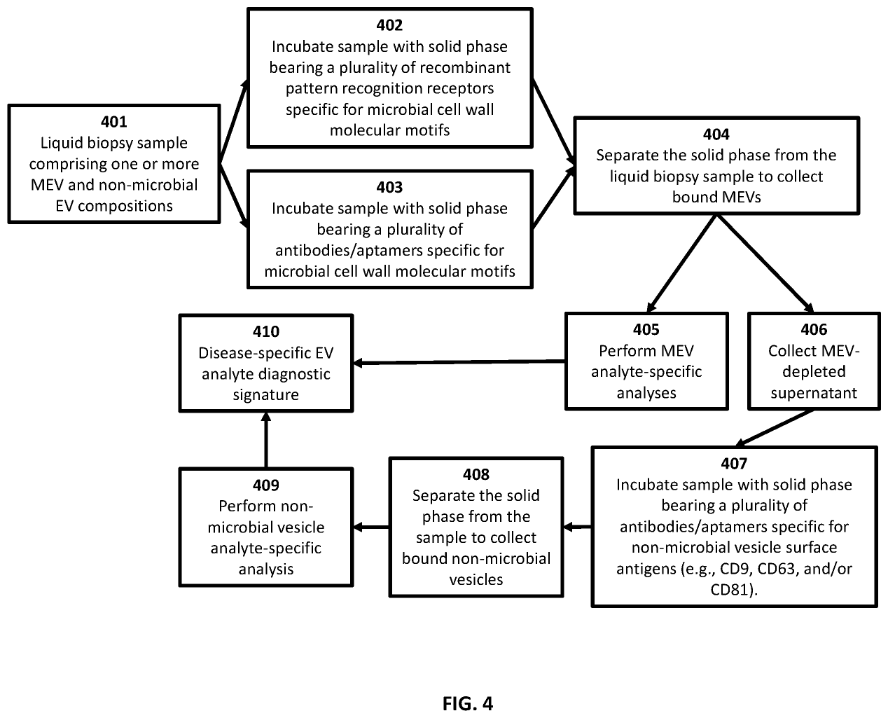

In some embodiments, a disease-specific diagnostic derived from the invention disclosed herein may comprise a combination of EV analyte data obtained from MEVs and non-microbial EVs, as shown in FIG. 4. In some instances, microbial analytes may be detected in non-microbial EVs. In some embodiments, the affinity-based enrichment of MEVs may be performed by incubating a liquid biological sample containing one or more MEV and non-microbial EV compositions 401 with one or more solid phase bearing MEV capture reagents. In some embodiments, the solid phase may be functionalized with recombinant pattern recognition receptors that may interact with microbial or non-microbial cell wall molecular motifs 402, which may generate a MEV affinity matrix. In some cases, the recombinant pattern recognition receptor may be specific to the microbial and/or non-microbial or non-microbial cell wall molecular motifs 402. In some embodiments, the solid phase may be functionalized with antibodies and/or aptamers that interact with the microbial and/or non-microbial or non-microbial cell wall molecular motifs 403, which may generate an MEV affinity matrix. In some cases, the antibodies and/or aptamers may have specificity for the microbial and/or non-microbial or non-microbial cell wall molecular motifs 403. In some embodiments, after incubation of the liquid biological sample 401 with the MEV affinity matrix, the MEV affinity matrix may be separated from the liquid biological sample 404 to collect some or all bound MEVs. In some embodiments, collection of MEV affinity matrix-bound MEVs may be followed by MEV analyte analysis 405 per the methods indicated in FIG. 1, 105-107. In some embodiments, separating the MEV affinity matrix from the liquid biological sample 404 may produce a MEV-depleted supernatant 406 containing non-microbial EVs. In some embodiments, the non-microbial EVs present in the MEV-depleted supernatant 406 may be isolated by incubating the MEV-depleted supernatant 406 with solid phase bearing antibodies and/or aptamers that interact with non-microbial vesicle surface antigens 407. In some cases, the antibodies and/or aptamers may be specific for the non-microbial vesicle surface antigens 407. In some embodiments, after incubation of the MEV-depleted supernatant 406 with the non-microbial EV affinity matrix, the non-microbial EV affinity matrix may be separated from the sample 408 to collect some or all bound non-microbial EVs. In some embodiments, collection of non-microbial EV affinity matrix-bound EVs may be followed by non-microbial EV analyte analysis 409 per the methods indicated in FIG. 1, 105-107. In some embodiments, data derived from MEV analyte-specific analyses 405 and non-microbial EV analyte-specific analyses 409 may be combined. In some cases, combining the data derived from MEV analyte-specific analyses 405 and non-microbial EV analyte-specific analyses 409 may form a signature of disease state, which may be a disease-specific EV analyte diagnostic signature 410.

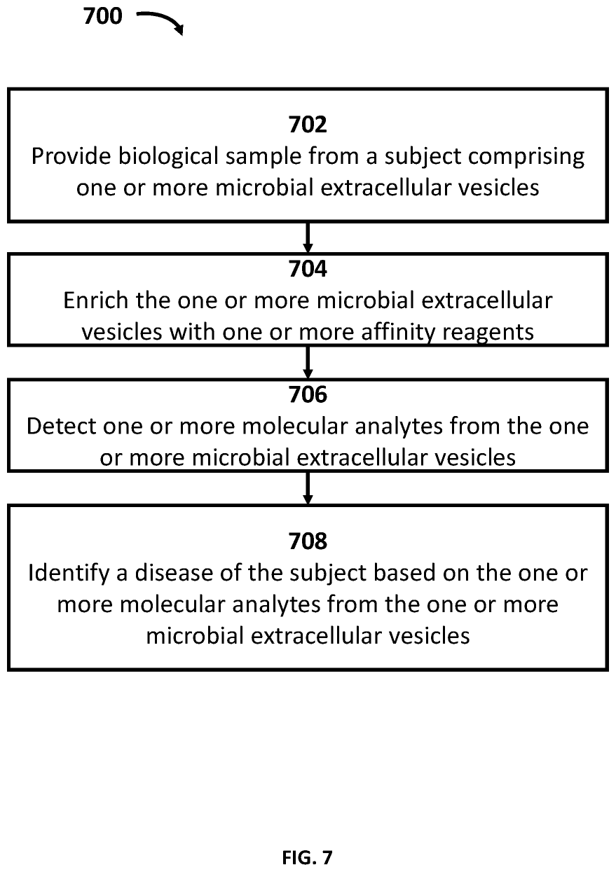

In some embodiments, the disclosure provided herein describes a method of identifying a disease of a subject 700, as seen in FIG. 7. In some cases, the method may comprise: (a) providing a biological sample from a subject comprising one or more microbial extracellular vesicles 702; (b) enriching the one or more microbial extracellular vesicles with one or more affinity reagents 704; (c) detecting one or more molecular analytes from the one or more microbial extracellular vesicles 706; (d) identifying the disease of the subject based on the one or more molecular analytes from the one or more microbial extracellular vesicles 708. In some cases, the biological sample may comprise one or more non-microbial extracellular vesicles and the one or more microbial extracellular vesicles. In some instances, the method may comprise quantifying an abundance of the one or more molecular analytes. The one or more molecular analytes of the one or more microbial extracellular vesicles may comprise vesicle-associated cell-free microbial DNA, cell-free microbial RNA, microbial DNA, microbial RNA microbial proteins, microbial metabolites, microbial lipids, microbial glycans, or any combination thereof. The one or more molecular analytes of the one or more non-microbial extracellular vesicles may comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. The biological sample may comprise a liquid biological sample, tissue biological sample, or any combination thereof. In some cases, the liquid biological sample may comprise plasma, serum, whole blood, urine, cerebral spinal fluid, saliva, sweat, tears, lymphatic fluid, exhaled breath condensate or any dilution, or processed fraction thereof. Whole blood may comprise plasma, white blood cells, red blood cells, platelets, or any combination thereof. In some cases, the subject may comprise a non-human mammal or a human subject. In some cases, the tissue biological sample may comprise tissue homogenate.

In some cases, the one or more affinity reagents may be configured to couple to one or more microbial and/or one or more non-microbial cell wall molecular motifs. The one or more microbial cell wall molecular motifs or the one or more non-microbial cell wall molecular motifs may comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof.

In some cases, the one or more affinity reagents may comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. The recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. The derivatives thereof may comprise an epitope tag and full-length recombinant innate immunity pattern recognition receptors or recombinant soluble ectodomains thereof. The epitope tag may comprise comprises a N- or C-terminal 6×-histidine tag, green fluorescent protein (GFP), myc, hemagglutinin (HA), Fc fusion, biotin, or any combination thereof. In some cases, the one or more affinity reagents may comprise a region to interact with the one or more microbial cell wall molecular motifs or the one or more non-microbial cell wall molecular motifs.

In some cases, the one or more antibodies, aptamers, single-chain variable fragments (scFV), or the single-chain antibodies may comprise an epitope tag. The epitope tag may comprise a N- or C-terminal 6×-histidine tag, green fluorescent protein (GFP), myc, hemagglutinin (HA), Fc fusion, biotin, or any combination thereof.

In some instances, enriching may comprise contact the biological sample with a support comprising covalently immobilized affinity agents. The covalently immobilized affinity agents may comprise a region that interacts with the one or more microbial cell wall molecular motifs or the one or more non-microbial cell wall molecular motifs. The support may comprise a magnetic bead, an agarose bead, non-magnetic latex, functionalized Sepharose, pH-sensitive polymers or any combination thereof.

In some cases, enriching may comprise: (a) contacting said biological sample with the one or more affinity reagents to form capture reagent-molecular motif interaction complexes; (b) contacting the capture reagent-molecular motif interaction complexes with a support; and (c) separating the support from the biological sample to concentrate said capture reagent-molecular motif interaction complexes. In some cases, the one or more affinity reagents may comprise an epitope tag. In some cases, the support may comprise an epitope tag recognition surface. The epitope tag recognition surface may comprise streptavidin, antibodies specific for 6×-histidine tag, GFP, myc, HA, biotin, or any combination thereof. The epitope tag recognition surface may comprise an anti-species antibody.

In some instances, detecting may comprise nucleic acid sequencing polymerase chain reaction (PCR), or hybridization-based analysis of the one or more molecular analytes. The nucleic acid sequencing may comprise whole genome sequencing, shotgun sequencing, next generation sequencing, targeted sequencing, RNA sequencing, methylation sequencing, or any combination thereof. In some cases, detecting may comprise performing mass spectrometry analyses, liquid chromatography-mass spectrometry (LC-MS), or high-performance liquid chromatography (HPLC). In some instances, detecting may comprise performing immunoassay analysis.

In some cases, the disease may comprise cancer. The cancer may comprise a stage I or stage II cancer. The cancer may comprise bone, breast, lung, colon, brain, skin, ovary, pancreas, or any combination thereof type of cancer. The cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprise one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, identifying the cancer may comprise comprises predicting the cancer by a predictive model, wherein the predictive model is trained with one or more subjects' one or more molecular analytes obtained from one or more microbial extracellular vesicles and an associated disease of the one more subjects. The predictive model may be configured to receive the one or more molecular analytes of the subject as an input and output the disease of the subject. The predictive model may be configured to predict one or more cancers, one or more subtypes of cancer, the anatomical location of one or more cancers, or any combination thereof. The predictive model may be configured to predict a stage of the cancer, prognosis of the cancer, mutation status of the cancer, future immunotherapy response of said cancer, an optimal therapy to treat said cancer, or any combination thereof. The predictive model may be configured to predict the cancer among one or more cancer types of the subject to identify a specific cancer type of the one or more cancer types.

In some cases, the predictive model may comprise a machine learning model, wherein the machine learning model comprises a regularized machine learning model, ensemble of machine learning models, or any combination thereof. The predictive model may comprise a random forest, neural network, naïve bayes, support vector machines, learning regression, k-nearest neighbors, k-means, decision tree, logistic regression, gradient boosting, or any combination thereof.

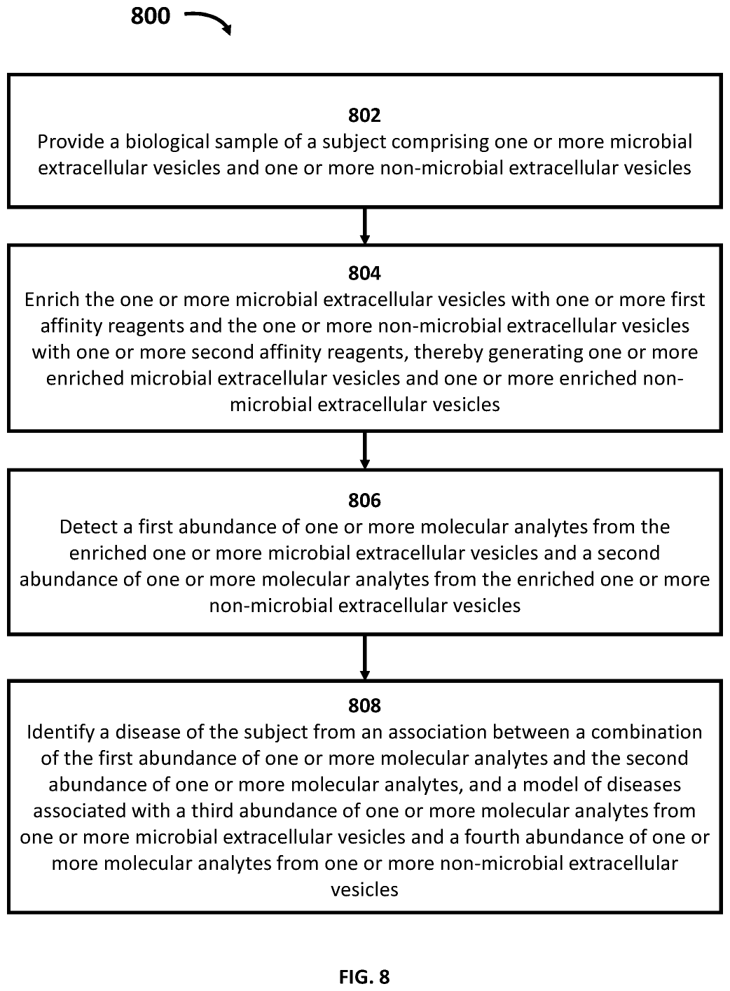

In some embodiments, the disclosure provided herein describes a method of identifying a disease of a subject 800, as seen in FIG. 8. In some cases, the method may comprise: (a) providing a biological sample of a subject comprising one or more microbial extracellular vesicles and one or more non-microbial extracellular vesicles 802; (b) enriching the one or more microbial extracellular vesicles with one or more first affinity reagents and the one or more non-microbial extracellular vesicles with one or more second affinity reagents, thereby generating one or more enriched microbial extracellular vesicles and one or more enriched non-microbial extracellular vesicles 804; (c) detecting a first abundance of one or more molecular analytes from the enriched one or more microbial extracellular vesicles and a second abundance of one or more molecular analytes from the enriched one or more non-microbial extracellular vesicles 806; and (d) identifying the disease of the subject from an association between a combination of the first abundance of one or more molecular analytes and the second abundance of one or more molecular analytes, and a model of diseases associated with a third abundance of one or more molecular analytes from one or more microbial extracellular vesicles and a fourth abundance of one or more molecular analytes from one or more non-microbial extracellular vesicles 808. In some cases, the third abundance and the fourth abundance may differ from the first abundance and the second abundance of one or more molecular analytes. In some instances, detecting may comprise quantifying the first abundance of one or more molecular analytes and the second abundance of one or more molecular analytes. The one or more molecular analytes of the one or more microbial extracellular vesicles may comprise cell-free microbial DNA, cell-free microbial RNA, microbial DNA, microbial RNA, microbial proteins, microbial metabolites, microbial lipids, microbial glycans, or any combination thereof. The one or more molecular analytes of the one or more non-microbial extracellular vesicles may comprise non-microbial cell-free DNA, non-microbial cell-free RNA, non-microbial DNA, non-microbial RNA, non-microbial proteins, non-microbial metabolites, non-microbial lipids, non-microbial glycans, or any combination thereof.

In some instances, the biological sample may comprise a liquid biological sample, a tissue biological sample, or any combination thereof. In some cases, the liquid biological sample may comprise plasma, serum, whole blood, urine, cerebral spinal fluid, saliva, sweat, tears, lymphatic fluid, exhaled breath condensate, or any combination, any dilution, or processed fraction thereof. Whole blood may comprise white blood cells, plasma, red blood cells, platelets, or any combination thereof. In some instances, the tissue biological sample may comprise tissue homogenate. In some cases, the subject may comprise a human or a non-human mammal.

In some cases, enriching may comprise concentrating one or more microbial cell wall molecular motifs or one or more non-microbial cell wall molecular motifs. The one or more microbial cell wall molecular motifs or the one or more non-microbial cell wall molecular motifs may comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof.

In some cases, the one or more first affinity reagents or the one or more second affinity reagents may comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof. The recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof. The derivatives thereof may comprise an epitope tag, full-length recombinant innate immunity pattern recognition receptors, or recombinant soluble ectodomains thereof. The one or more antibodies, the aptamers, the single-chain variable fragments (scFV), and the single-chain antibodies may comprise an epitope tag. The epitope tag may comprise a N- or C-terminal 6×-histidine tag, green fluorescent protein (GFP), myc, hemagglutinin (HA), Fc fusion, biotin, or any combination thereof. In some cases, the one or more first affinity reagents and the one or more second affinity reagents may comprise a region to interact with the one or more microbial cell wall molecular motifs or the one or more non-microbial cell wall molecular motifs. In some cases, the one or more first affinity reagents or the one or more second affinity reagents may be specific to mammalian antigens CD9, CD63, CD81, glypican 1 (GPC1), Mart-1, TYRP2, Human epidermal growth factor receptors (HER) family members, EpCAM, or any combination thereof.

In some cases, enriching may comprise incubating the biological sample with a support comprising covalently immobilized affinity agents. The covalently immobilized affinity agents may comprise a region that interacts with the one or more microbial cell wall molecular motifs or the one or more non-microbial cell wall molecular motifs. In some instances, the support may comprise a magnetic bead, an agarose bead, non-magnetic latex, functionalized Sepharose, pH-sensitive polymers or any combination thereof.

In some cases, detecting may comprise performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with the one or more molecular analytes of the one or more microbial extracellular vesicles or the one or more molecular analytes from the one or more non-microbial extracellular vesicles. The nucleic acid sequencing may comprise whole genome sequencing, shotgun sequencing, next generations sequencing, targeted sequencing, RNA sequencing, methylation sequencing, or any combination thereof.

In some cases, the model may comprise a predictive mode, where the predictive model may be trained with one or more subjects' third abundance of one or more molecular analytes from the one or more microbial extracellular vesicles, fourth abundance of one or more molecular analytes from the one or more non-microbial extracellular vesicles, and a corresponding disease of the one or more subjects. In some instances, the disease may comprise cancer, described elsewhere herein. In some cases, the predictive model may be configured to receive the first abundance of one or more molecular analytes and the second abundance of one or more molecular analytes as an input and output a prediction of the disease of the subject. In some instances, the predictive model may be configured to predict one or more cancers, one or more subtypes of cancer, anatomical locations of the one or more cancers, or any combination thereof in the subject.

In some instances, the disease may comprise cancer. The cancer may comprise a stage I or stage II cancer. In some instances, the cancer may comprise bone, breast, lung, colon, brain, skin, ovary, pancreas, or any combination thereof type of cancer. In some cases, the cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprise one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, the predictive model may comprise a machine learning model. The machine learning model may comprise one or more machine learning models, a regularized machine learning model, an ensemble of machine learning models, or any combination thereof. In some instances, the predictive model may comprise a random forest, neural network, naïve bayes, support vector machines, linear regression, k-nearest neighbors, k-means, decision tree, logistic regression, gradient boosting, or any combination thereof predictive model. In some cases, enriching the one or more microbial extracellular vesicles and the one or more non-microbial extracellular vesicles may improve an accuracy of the predictive model by at least 1%, at least 5%, at least 10%, at least 15%, or at least 20% when predicting the cancer of the subject. In some instances, an area under a receiver operating characteristic curve of the predictive model when predicting the disease of the subject is increased by at 1%, at least 2%, at least 4%, at least 5%, or at least 10%, when said combination of said first abundance of one or more molecular analytes and said second abundance of one or more molecular analytes are provided as said input to said predictive model.

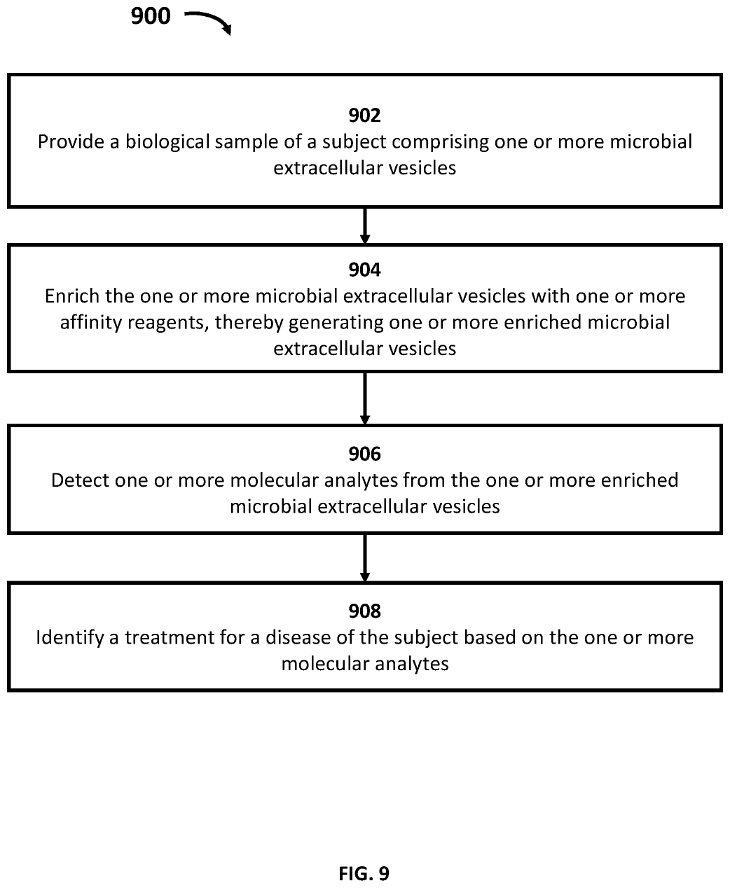

In some embodiments, the disclosure provided herein describes a method of identifying a treatment for a disease of a subject 900, as seen in FIG. 9. In some cases, the method may comprise: (a) providing a biological sample from a subject comprising one or more microbial extracellular vesicles 902; (b) enriching the one or more microbial extracellular vesicles with one or more affinity reagents 904; (c) detecting one or more molecular analytes from the one or more microbial extracellular vesicles 906; (d) identifying the a treatment for a disease of the subject based on the one or more molecular analytes from the one or more microbial extracellular vesicles 908. In some cases, the biological sample may comprise one or more non-microbial extracellular vesicles and the one or more microbial extracellular vesicles. In some instances, the method may comprise quantifying an abundance of the one or more molecular analytes. The one or more molecular analytes of the one or more microbial extracellular vesicles may comprise vesicle-associated cell-free microbial DNA, cell-free microbial RNA, microbial DNA, microbial RNA microbial proteins, microbial metabolites, microbial lipids, microbial glycans, or any combination thereof. The one or more molecular analytes of the one or more non-microbial extracellular vesicles may comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. In some instances, the biological sample may comprise a liquid biological sample, tissue biological sample, or any combination thereof. In some cases, the liquid biological sample may comprise plasma, serum, whole blood, urine, cerebral spinal fluid, saliva, sweat, tears, lymphatic fluid, exhaled breath condensate or any dilution, or processed fraction thereof. Whole blood may comprise plasma, white blood cells, red blood cells, platelets, or any combination thereof. In some cases, the subject may comprise a non-human mammal or a human subject. In some cases, the tissue biological sample comprises tissue homogenate.

In some cases, the one or more affinity reagents may be configured to couple to one or more microbial and/or one or more non-microbial cell wall molecular motifs. The one or more microbial cell wall molecular motifs or the one or more non-microbial cell wall molecular motifs may comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof.

In some cases, the one or more affinity reagents may comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. The recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. The derivatives thereof may comprise an epitope tag and full-length recombinant innate immunity pattern recognition receptors or recombinant soluble ectodomains thereof. The epitope tag may comprise comprises a N- or C-terminal 6×-histidine tag, green fluorescent protein (GFP), myc, hemagglutinin (HA), Fc fusion, biotin, or any combination thereof. In some cases, the one or more affinity reagents may comprise a region to interact with the one or more microbial cell wall molecular motifs or the one or more non-microbial cell wall molecular motifs.

In some cases, the one or more antibodies, aptamers, single-chain variable fragments (scFV), or the single-chain antibodies may comprise an epitope tag. The epitope tag may comprise a N- or C-terminal 6×-histidine tag, green fluorescent protein (GFP), myc, hemagglutinin (HA), Fc fusion, biotin, or any combination thereof.

In some instances, enriching may comprise contacting the biological sample with a support comprising covalently immobilized affinity agents. The covalently immobilized affinity agents may comprise a region that interacts with the one or more microbial cell wall molecular motifs or the one or more non-microbial cell wall molecular motifs. The support may comprise a magnetic bead, an agarose bead, non-magnetic latex, functionalized Sepharose, pH-sensitive polymers or any combination thereof.

In some cases, enriching may comprise: (a) contacting said biological sample with the one or more affinity reagents to form capture reagent-molecular motif interaction complexes; (b) contacting the capture reagent-molecular motif interaction complexes with a support; and (c) separating the support from the biological sample to concentrate said capture reagent-molecular motif interaction complexes. In some cases, the one or more affinity reagents may comprise an epitope tag. In some cases, the support may comprise an epitope tag recognition surface. The epitope tag recognition surface may comprise streptavidin, antibodies specific for 6×-histidine tag, GFP, myc, HA, biotin, or any combination thereof. The epitope tag recognition surface may comprise an anti-species antibody.

In some instances, detecting may comprise nucleic acid sequencing polymerase chain reaction (PCR), or hybridization-based analysis of the one or more molecular analytes. The nucleic acid sequencing may comprise whole genome sequencing, shotgun sequencing, next generation sequencing, targeted sequencing, RNA sequencing, methylation sequencing, or any combination thereof. In some cases, detecting may comprise performing mass spectrometry analyses, liquid chromatography-mass spectrometry (LC-MS), or high-performance liquid chromatography (HPLC). In some instances, detecting may comprise performing immunoassay analysis.

In some cases, the disease may comprise cancer. The cancer may comprise a stage I or stage II cancer. The cancer may comprise bone, breast, lung, colon, brain, skin, ovary, pancreas, or any combination thereof type of cancer. The cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprise one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, identifying the treatment for the disease may comprise comprises predicting the treatment by a predictive model, wherein the predictive model is trained with one or more subjects' one or more molecular analytes obtained from one or more microbial extracellular vesicles and an associated treatment for the disease of the one more subjects. The predictive model may be configured to receive the one or more molecular analytes of the subject as an input and output the treatment for the disease of the subject.

In some cases, the predictive model may comprise a machine learning model, wherein the machine learning model comprises a regularized machine learning model, ensemble of machine learning models, or any combination thereof. The predictive model may comprise a random forest, neural network, naïve bayes, support vector machines, learning regression, k-nearest neighbors, k-means, decision tree, logistic regression, gradient boosting, or any combination thereof.

In some cases, enriching the one or more microbial extracellular vesicles may improve an accuracy of the predictive model by at least 1%, at least 5%, at least 10%, at least 15%, or at least 20% when predicting said treatment of said subject.

In some cases, the treatment may comprise a repurposed treatment, which may or may not have been originally approved for targeting cancer. In some cases, the treatment may comprise a small molecule, a biologic, a probiotic, a virus, a bacteriophage, an immunotherapy, a broad-spectrum antibiotic, or any combination thereof. The probiotic may comprise an engineered bacterium strain or ensemble of engineered bacteria. In some cases, the treatment may comprise an adjuvant given in combination with a primary treatment against the subject's cancer to improve an efficacy of the primary treatment. In some cases, the treatment may comprise adoptive cell transfer to target microbial antigens associated with said cancer or cancer microenvironment. The treatment may comprise a cancer vaccine that exploits microbial antigens associated with the cancer or cancer microenvironment. The treatment may comprise a monoclonal antibody against microbial antigens associated with the cancer or cancer microenvironment. The treatment may comprise an antibody-drug conjugate designed to at least partially target microbial antigens associated with the cancer or cancer microenvironment. The treatment may comprise a multi-valent antibody, antibody fragment, or antibody derivative thereof designed to at least partially target one or more microbial antigens associated with the cancer or cancer microenvironment. The treatment may comprise a targeted antibiotic against a particular kind of microbe or class of functionally or biologically similar microbes. In some cases, two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

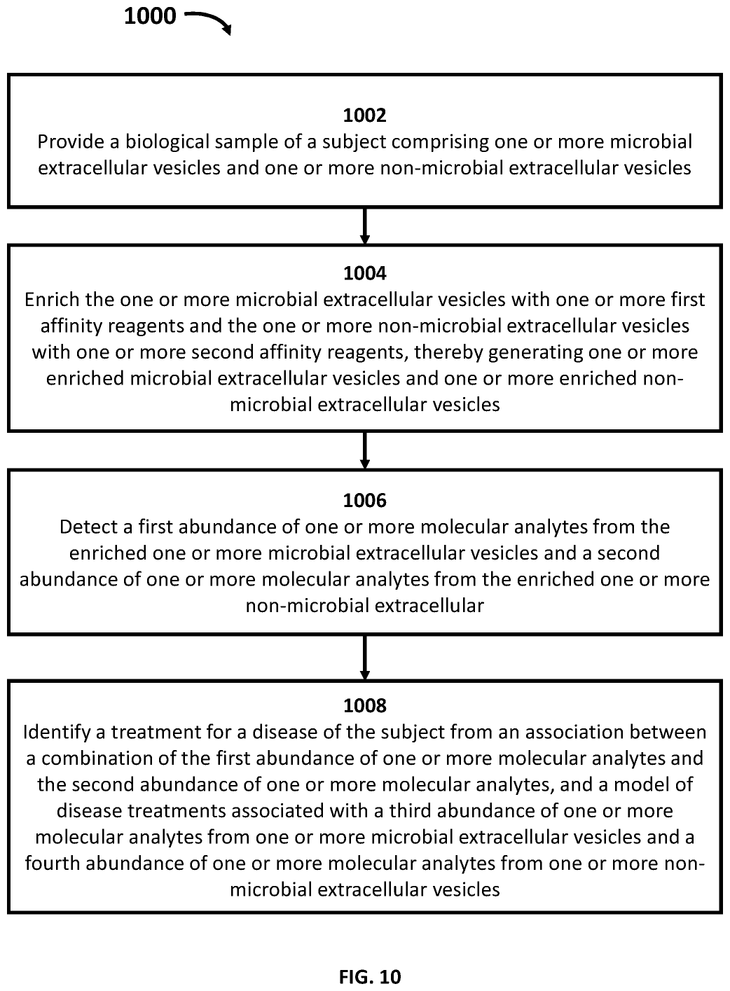

In some embodiments, the disclosure provided herein describes a method of identifying a treatment for a disease of a subject 1000, as seen in FIG. 10. In some cases, the method may comprise: (a) providing a biological sample of a subject comprising one or more microbial extracellular vesicles and one or more non-microbial extracellular vesicles 1002; (b) enriching the one or more microbial extracellular vesicles with one or more first affinity reagents and the one or more non-microbial extracellular vesicles with one or more second affinity reagents, thereby generating one or more enriched microbial extracellular vesicles and one or more enriched non-microbial extracellular vesicles 1004; (c) detecting a first abundance of one or more molecular analytes from the enriched one or more microbial extracellular vesicles and a second abundance of one or more molecular analytes from the enriched one or more non-microbial extracellular vesicles 1006; and (d) identifying a treatment for the disease of the subject from an association between a combination of the first abundance of one or more molecular analytes and the second abundance of one or more molecular analytes, and a model of disease treatments associated with a third abundance of one or more molecular analytes from one or more microbial extracellular vesicles and a fourth abundance of one or more molecular analytes from one or more non-microbial extracellular vesicles 1008. In some cases, the third abundance and the fourth abundance may differ from the first abundance and the second abundance of one or more molecular analytes. In some instances, detecting may comprise quantifying the first abundance of one or more molecular analytes and the second abundance of one or more molecular analytes. The one or more molecular analytes of the one or more microbial extracellular vesicles may comprise cell-free microbial DNA, cell-free microbial RNA, microbial DNA, microbial RNA, microbial proteins, microbial metabolites, microbial lipids, microbial glycans, or any combination thereof. The one or more molecular analytes of the one or more non-microbial extracellular vesicles may comprise non-microbial cell-free DNA, non-microbial cell-free RNA, non-microbial DNA, non-microbial RNA, non-microbial proteins, non-microbial metabolites, non-microbial lipids, non-microbial glycans, or any combination thereof.

In some instances, the biological sample may comprise a liquid biological sample, a tissue biological sample, or any combination thereof. In some cases, the liquid biological sample may comprise plasma, serum, whole blood, urine, cerebral spinal fluid, saliva, sweat, tears, lymphatic fluid, exhaled breath condensate, or any combination, any dilution, or processed fraction thereof. Whole blood may comprise white blood cells, plasma, red blood cells, platelets, or any combination thereof. In some instances, the tissue biological sample may comprise tissue homogenate. In some cases, the subject may comprise a human or a non-human mammal.

In some cases, enriching may comprise concentrating one or more microbial cell wall molecular motifs or one or more non-microbial cell wall molecular motifs. The one or more microbial cell wall molecular motifs or the one or more non-microbial cell wall molecular motifs may comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof.

In some cases, the one or more first affinity reagents or the one or more second affinity reagents may comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof. The recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof. The derivatives thereof may comprise an epitope tag, full-length recombinant innate immunity pattern recognition receptors, or recombinant soluble ectodomains thereof. The one or more antibodies, the aptamers, the single-chain variable fragments (scFV), and the single-chain antibodies may comprise an epitope tag. The epitope tag may comprise a N- or C-terminal 6×-histidine tag, green fluorescent protein (GFP), myc, hemagglutinin (HA), Fc fusion, biotin, or any combination thereof. In some cases, the one or more first affinity reagents and the one or more second affinity reagents may comprise a region to interact with the one or more microbial cell wall molecular motifs or the one or more non-microbial cell wall molecular motifs. In some cases, the one or more first affinity reagents or the one or more second affinity reagents may be specific to mammalian antigens CD9, CD63, CD81, glypican 1 (GPC1), Mart-1, TYRP2, Human epidermal growth factor receptors (HER) family members, EpCAM, or any combination thereof.

In some cases, enriching may comprise incubating the biological sample with a support comprising covalently immobilized affinity agents. The covalently immobilized affinity agents may comprise a region that interacts with the one or more microbial cell wall molecular motifs or the one or more non-microbial cell wall molecular motifs. In some instances, the support may comprise a magnetic bead, an agarose bead, non-magnetic latex, functionalized Sepharose, pH-sensitive polymers or any combination thereof.

In some cases, detecting may comprise performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with the one or more molecular analytes of the one or more microbial extracellular vesicles or the one or more molecular analytes from the one or more non-microbial extracellular vesicles. The nucleic acid sequencing may comprise whole genome sequencing, shotgun sequencing, next generations sequencing, targeted sequencing, RNA sequencing, methylation sequencing, or any combination thereof.

In some cases, the model may comprise a predictive mode, where the predictive model may be trained with one or more subjects' third abundance of one or more molecular analytes from the one or more microbial extracellular vesicles, fourth abundance of one or more molecular analytes from the one or more non-microbial extracellular vesicles, and a corresponding treatment for the disease of the one or more subjects. In some instances, the disease may comprise cancer, described elsewhere herein. In some cases, the predictive model may be configured to receive the first abundance of one or more molecular analytes and the second abundance of one or more molecular analytes as an input and output a prediction of the treatment for the disease of the subject. In some instances, the predictive model may be configured to predict one or more cancers, one or more subtypes of cancer, anatomical locations of the one or more cancers, or any combination thereof in the subject.

In some instances, the disease may comprise cancer. The cancer may comprise a stage I or stage II cancer. In some instances, the cancer may comprise bone, breast, lung, colon, brain, skin, ovary, pancreas, or any combination thereof type of cancer. In some cases, the cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprise one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, the predictive model may comprise a machine learning model. The machine learning model may comprise one or more machine learning models, a regularized machine learning model, an ensemble of machine learning models, or any combination thereof. In some instances, the predictive model may comprise a random forest, neural network, naïve bayes, support vector machines, linear regression, k-nearest neighbors, k-means, decision tree, logistic regression, gradient boosting, or any combination thereof predictive model. In some cases, enriching the one or more microbial extracellular vesicles and the one or more non-microbial extracellular vesicles may improve an accuracy of the predictive model by at least 1%, at least 5%, at least 10%, at least 15%, or at least 20% when predicting the cancer of the subject. In some instances, an area under a receiver operating characteristic curve of the predictive model when predicting the treatment for the disease of the subject is increased by at 1%, at least 2%, at least 4%, at least 5%, or at least 10%, when said combination of said first abundance of one or more molecular analytes and said second abundance of one or more molecular analytes are provided as said input to said predictive model.

In some cases, the treatment may comprise a repurposed treatment, which may or may not have been originally approved for targeting cancer. In some cases, the treatment may comprise a small molecule, a biologic, a probiotic, a virus, a bacteriophage, an immunotherapy, a broad-spectrum antibiotic, or any combination thereof. The probiotic may comprise an engineered bacterium strain or ensemble of engineered bacteria. In some cases, the treatment may comprise an adjuvant given in combination with a primary treatment against the subject's cancer to improve an efficacy of the primary treatment. In some cases, the treatment may comprise adoptive cell transfer to target microbial antigens associated with said cancer or cancer microenvironment. The treatment may comprise a cancer vaccine that exploits microbial antigens associated with the cancer or cancer microenvironment. The treatment may comprise a monoclonal antibody against microbial antigens associated with the cancer or cancer microenvironment. The treatment may comprise an antibody-drug conjugate designed to at least partially target microbial antigens associated with the cancer or cancer microenvironment. The treatment may comprise a multi-valent antibody, antibody fragment, or antibody derivative thereof designed to at least partially target one or more microbial antigens associated with the cancer or cancer microenvironment. The treatment may comprise a targeted antibiotic against a particular kind of microbe or class of functionally or biologically similar microbes. In some cases, two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

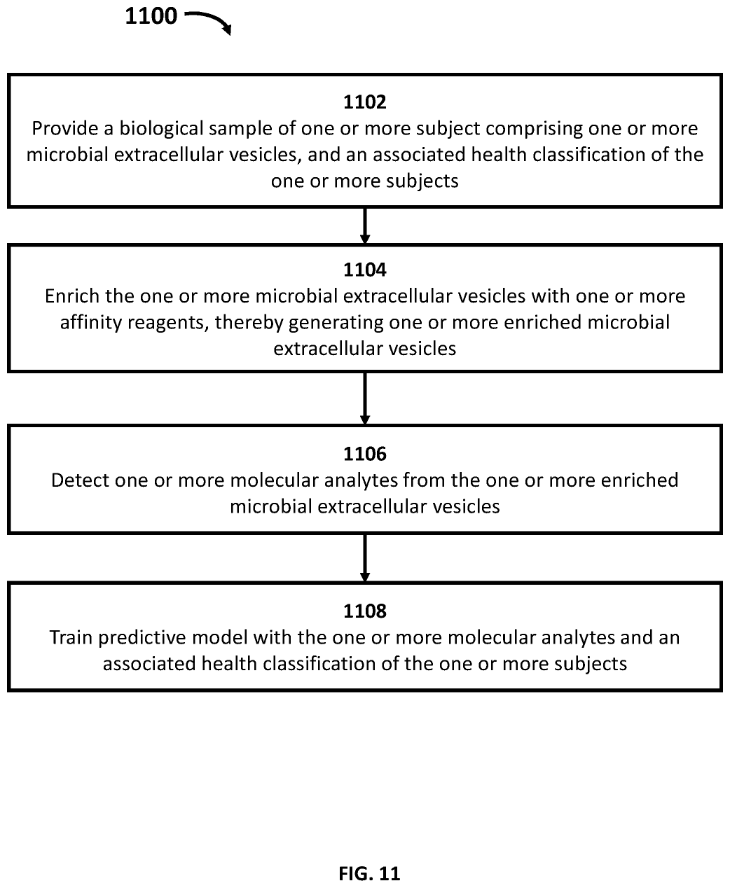

In some embodiments, the disclosure provided herein describes a method training a predictive model 1100, as seen in FIG. 11. In some cases, the method may comprise: (a) providing a biological sample from one or more subjects comprising one or more microbial extracellular vesicles, and an associated health classification of the one or more subjects 1102; (b) enriching the one or more microbial extracellular vesicles with one or more affinity reagents, thereby generating one or more enriched microbial extracellular vesicles 1104; (c) detecting one or more molecular analytes from the one or more enriched microbial extracellular vesicles 1106; (d) training a predictive model with the one or more molecular analytes and an associated health classification of the one or more subjects 1108. In some instances, the trained predictive model may be configured to receive one or more molecular analytes as an input and output a predicted disease of the subject, a treatment for the disease of the subject, or any combination thereof. In some cases, the associated health classification of the one or more subjects may comprise cancerous, non-cancerous disease, non-cancerous and not diseased (e.g., healthy).

In some cases, the biological sample may comprise one or more non-microbial extracellular vesicles and the one or more microbial extracellular vesicles. In some instances, the method may comprise quantifying an abundance of the one or more molecular analytes. The one or more molecular analytes of the one or more microbial extracellular vesicles may comprise vesicle-associated cell-free microbial DNA, cell-free microbial RNA, microbial DNA, microbial RNA microbial proteins, microbial metabolites, microbial lipids, microbial glycans, or any combination thereof. The one or more molecular analytes of the one or more non-microbial extracellular vesicles may comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. In some instances, the biological sample may comprise a liquid biological sample, tissue biological sample, or any combination thereof. In some cases, the liquid biological sample may comprise plasma, serum, whole blood, urine, cerebral spinal fluid, saliva, sweat, tears, lymphatic fluid, exhaled breath condensate or any dilution, or processed fraction thereof. Whole blood may comprise plasma, white blood cells, red blood cells, platelets, or any combination thereof. In some cases, the subject may comprise a non-human mammal or a human subject. In some cases, the tissue biological sample comprises tissue homogenate.

In some cases, the one or more affinity reagents may be configured to couple to one or more microbial and/or one or more non-microbial cell wall molecular motifs. The one or more microbial cell wall molecular motifs or the one or more non-microbial cell wall molecular motifs may comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof.

In some cases, the one or more affinity reagents may comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. The recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. The derivatives thereof may comprise an epitope tag and full-length recombinant innate immunity pattern recognition receptors or recombinant soluble ectodomains thereof. The epitope tag may comprise comprises a N- or C-terminal 6×-histidine tag, green fluorescent protein (GFP), myc, hemagglutinin (HA), Fc fusion, biotin, or any combination thereof. In some cases, the one or more affinity reagents may comprise a region to interact with the one or more microbial cell wall molecular motifs or the one or more non-microbial cell wall molecular motifs.

In some cases, the one or more antibodies, aptamers, single-chain variable fragments (scFV), or the single-chain antibodies may comprise an epitope tag. The epitope tag may comprise a N- or C-terminal 6×-histidine tag, green fluorescent protein (GFP), myc, hemagglutinin (HA), Fc fusion, biotin, or any combination thereof.

In some instances, enriching may comprise contacting the biological sample with a support comprising covalently immobilized affinity agents. The covalently immobilized affinity agents may comprise a region that interacts with the one or more microbial cell wall molecular motifs or the one or more non-microbial cell wall molecular motifs. The support may comprise a magnetic bead, an agarose bead, non-magnetic latex, functionalized Sepharose, pH-sensitive polymers or any combination thereof.

In some cases, enriching may comprise: (a) contacting said biological sample with the one or more affinity reagents to form capture reagent-molecular motif interaction complexes; (b) contacting the capture reagent-molecular motif interaction complexes with a support; and (c) separating the support from the biological sample to concentrate said capture reagent-molecular motif interaction complexes. In some cases, the one or more affinity reagents may comprise an epitope tag. In some cases, the support may comprise an epitope tag recognition surface. The epitope tag recognition surface may comprise streptavidin, antibodies specific for 6×-histidine tag, GFP, myc, HA, biotin, or any combination thereof. The epitope tag recognition surface may comprise an anti-species antibody.

In some instances, detecting may comprise nucleic acid sequencing polymerase chain reaction (PCR), or hybridization-based analysis of the one or more molecular analytes. The nucleic acid sequencing may comprise whole genome sequencing, shotgun sequencing, next generation sequencing, targeted sequencing, RNA sequencing, methylation sequencing, or any combination thereof. In some cases, detecting may comprise performing mass spectrometry analyses, liquid chromatography-mass spectrometry (LC-MS), or high-performance liquid chromatography (HPLC). In some instances, detecting may comprise performing immunoassay analysis.

In some cases, the disease may comprise cancer. The cancer may comprise a stage I or stage II cancer. The cancer may comprise bone, breast, lung, colon, brain, skin, ovary, pancreas, or any combination thereof type of cancer. The cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprise one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, the trained predictive model may be configured to predict one or more cancers, one or more subtypes of cancer, the anatomical location of one or more cancers, or any combination thereof one or more subjects. The trained predictive model may be configured to predict a stage of the cancer, prognosis of the cancer, mutation status of the cancer, future immunotherapy response of the cancer, an optimal therapy to treat the cancer, or any combination thereof. The trained predictive model may be configured to predict the cancer among one or more cancer types of the one or more subjects to identify a specific cancer type of the one or more cancer types. In some cases, the trained predictive model may comprise a machine learning model, wherein the machine learning model comprises a regularized machine learning model, ensemble of machine learning models, or any combination thereof. The trained predictive model may comprise a random forest, neural network, naïve bayes, support vector machines, learning regression, k-nearest neighbors, k-means, decision tree, logistic regression, gradient boosting, or any combination thereof.

In some cases, enriching the one or more microbial extracellular vesicles may improve an accuracy of the trained predictive model by at least 1%, at least 5%, at least 10%, at least 15%, or at least 20% when predicting said treatment of said subject or said disease of said subject.

In some cases, the treatment may comprise a repurposed treatment, which may or may not have been originally approved for targeting cancer. In some cases, the treatment may comprise a small molecule, a biologic, a probiotic, a virus, a bacteriophage, an immunotherapy, a broad-spectrum antibiotic, or any combination thereof. The probiotic may comprise an engineered bacterium strain or ensemble of engineered bacteria. In some cases, the treatment may comprise an adjuvant given in combination with a primary treatment against the subject's cancer to improve an efficacy of the primary treatment. In some cases, the treatment may comprise adoptive cell transfer to target microbial antigens associated with said cancer or cancer microenvironment. The treatment may comprise a cancer vaccine that exploits microbial antigens associated with the cancer or cancer microenvironment. The treatment may comprise a monoclonal antibody against microbial antigens associated with the cancer or cancer microenvironment. The treatment may comprise an antibody-drug conjugate designed to at least partially target microbial antigens associated with the cancer or cancer microenvironment. The treatment may comprise a multi-valent antibody, antibody fragment, or antibody derivative thereof designed to at least partially target one or more microbial antigens associated with the cancer or cancer microenvironment. The treatment may comprise a targeted antibiotic against a particular kind of microbe or class of functionally or biologically similar microbes. In some cases, two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

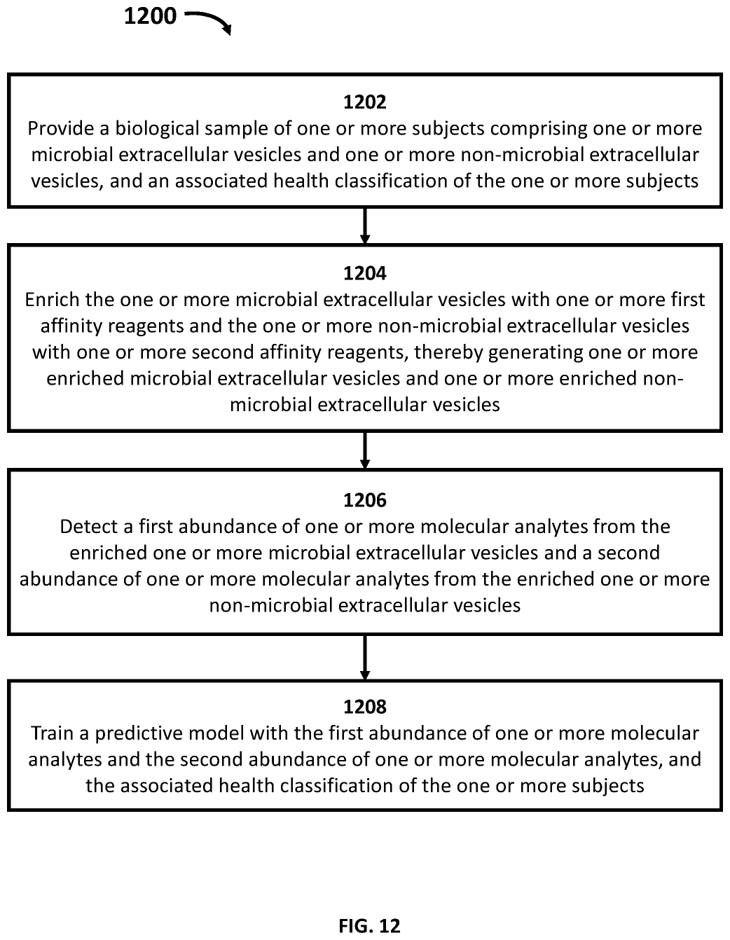

In some embodiments, the disclosure provided herein describes a method of training a predictive model 1200, as seen in FIG. 12. In some cases, the method may comprise: (a) providing a biological sample of one or more subjects comprising one or more microbial extracellular vesicles and one or more non-microbial extracellular vesicles, and an associated health classification of the one or more subjects 1202; (b) enriching the one or more microbial extracellular vesicles with one or more first affinity reagents and the one or more non-microbial extracellular vesicles with one or more second affinity reagents, thereby generating one or more enriched microbial extracellular vesicles and one or more enriched non-microbial extracellular vesicles 1204; (c) detecting a first abundance of one or more molecular analytes from the enriched one or more microbial extracellular vesicles and a second abundance of one or more molecular analytes from the enriched one or more non-microbial extracellular vesicles 1206; and (d) training a predictive model with the first abundance of one or more molecular analytes and the second abundance of one or more molecular analytes, and the associated health classifications of the one or more subjects 1208. In some instances, detecting may comprise quantifying the first abundance of one or more molecular analytes and the second abundance of one or more molecular analytes. In some instances, the trained predictive model may be configured to receive the first abundance of one or more molecular analytes and the second abundance of one or more molecular analytes as an input and output a predicted disease of the subject, a treatment for the disease of the subject, or any combination thereof. In some cases, the associated health classification of the one or more subjects may comprise cancerous, non-cancerous disease, non-cancerous and not diseased (e.g., healthy).

The one or more molecular analytes of the one or more microbial extracellular vesicles may comprise cell-free microbial DNA, cell-free microbial RNA, microbial DNA, microbial RNA, microbial proteins, microbial metabolites, microbial lipids, microbial glycans, or any combination thereof. The one or more molecular analytes of the one or more non-microbial extracellular vesicles may comprise non-microbial cell-free DNA, non-microbial cell-free RNA, non-microbial DNA, non-microbial RNA, non-microbial proteins, non-microbial metabolites, non-microbial lipids, non-microbial glycans, or any combination thereof.

In some instances, the biological sample may comprise a liquid biological sample, a tissue biological sample, or any combination thereof. In some cases, the liquid biological sample may comprise plasma, serum, whole blood, urine, cerebral spinal fluid, saliva, sweat, tears, lymphatic fluid, exhaled breath condensate, or any combination, any dilution, or processed fraction thereof. Whole blood may comprise white blood cells, plasma, red blood cells, platelets, or any combination thereof. In some instances, the tissue biological sample may comprise tissue homogenate. In some cases, the subject may comprise a human or a non-human mammal.

In some cases, enriching may comprise concentrating one or more microbial cell wall molecular motifs or one or more non-microbial cell wall molecular motifs. The one or more microbial cell wall molecular motifs or the one or more non-microbial cell wall molecular motifs may comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof.

In some cases, the one or more first affinity reagents or the one or more second affinity reagents may comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof. The recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof. The derivatives thereof may comprise an epitope tag, full-length recombinant innate immunity pattern recognition receptors, or recombinant soluble ectodomains thereof. The one or more antibodies, the aptamers, the single-chain variable fragments (scFV), and the single-chain antibodies may comprise an epitope tag. The epitope tag may comprise a N- or C-terminal 6×-histidine tag, green fluorescent protein (GFP), myc, hemagglutinin (HA), Fc fusion, biotin, or any combination thereof. In some cases, the one or more first affinity reagents and the one or more second affinity reagents may comprise a region to interact with the one or more microbial cell wall molecular motifs or the one or more non-microbial cell wall molecular motifs. In some cases, the one or more first affinity reagents or the one or more second affinity reagents may be specific to mammalian antigens CD9, CD63, CD81, glypican 1 (GPC1), Mart-1, TYRP2, Human epidermal growth factor receptors (HER) family members, EpCAM, or any combination thereof.

In some cases, enriching may comprise incubating the biological sample with a support comprising covalently immobilized affinity agents. The covalently immobilized affinity agents may comprise a region that interacts with the one or more microbial cell wall molecular motifs or the one or more non-microbial cell wall molecular motifs. In some instances, the support may comprise a magnetic bead, an agarose bead, non-magnetic latex, functionalized Sepharose, pH-sensitive polymers or any combination thereof.

In some cases, detecting may comprise performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with the one or more molecular analytes of the one or more microbial extracellular vesicles or the one or more molecular analytes from the one or more non-microbial extracellular vesicles. The nucleic acid sequencing may comprise whole genome sequencing, shotgun sequencing, next generations sequencing, targeted sequencing, RNA sequencing, methylation sequencing, or any combination thereof.

In some instances, the predictive model may be configured to predict one or more cancers, one or more subtypes of cancer, anatomical locations of the one or more cancers, or any combination thereof one or more subjects.

In some instances, the disease may comprise cancer. The cancer may comprise a stage I or stage II cancer. In some instances, the cancer may comprise bone, breast, lung, colon, brain, skin, ovary, pancreas, or any combination thereof type of cancer. In some cases, the cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprise one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, the trained predictive model may be configured to predict one or more cancers, one or more subtypes of cancer, the anatomical location of one or more cancers, or any combination thereof one or more subjects. The trained predictive model may be configured to predict a stage of the cancer, prognosis of the cancer, mutation status of the cancer, future immunotherapy response of the cancer, an optimal therapy to treat the cancer, or any combination thereof. The trained predictive model may be configured to predict the cancer among one or more cancer types of the one or more subjects to identify a specific cancer type of the one or more cancer types. In some cases, the trained predictive model may comprise a machine learning model. The machine learning model may comprise one or more machine learning models, a regularized machine learning model, an ensemble of machine learning models, or any combination thereof. In some instances, the trained predictive model may comprise a random forest, neural network, naïve bayes, support vector machines, linear regression, k-nearest neighbors, k-means, decision tree, logistic regression, gradient boosting, or any combination thereof predictive model. In some cases, enriching the one or more microbial extracellular vesicles and the one or more non-microbial extracellular vesicles may improve an accuracy of the trained predictive model by at least 1%, at least 5%, at least 10%, at least 15%, or at least 20% when predicting the cancer of the subject. In some instances, an area under a receiver operating characteristic curve of the trained predictive model when predicting the treatment for the disease of one or more subjects is increased by at 1%, at least 2%, at least 4%, at least 5%, or at least 10%, when said combination of said first abundance of one or more molecular analytes and said second abundance of one or more molecular analytes are provided as said input to said trained predictive model.

In some cases, the treatment may comprise a repurposed treatment, which may or may not have been originally approved for targeting cancer. In some cases, the treatment may comprise a small molecule, a biologic, a probiotic, a virus, a bacteriophage, an immunotherapy, a broad-spectrum antibiotic, or any combination thereof. The probiotic may comprise an engineered bacterium strain or ensemble of engineered bacteria. In some cases, the treatment may comprise an adjuvant given in combination with a primary treatment against the subject's cancer to improve an efficacy of the primary treatment. In some cases, the treatment may comprise adoptive cell transfer to target microbial antigens associated with said cancer or cancer microenvironment. The treatment may comprise a cancer vaccine that exploits microbial antigens associated with the cancer or cancer microenvironment. The treatment may comprise a monoclonal antibody against microbial antigens associated with the cancer or cancer microenvironment. The treatment may comprise an antibody-drug conjugate designed to at least partially target microbial antigens associated with the cancer or cancer microenvironment. The treatment may comprise a multi-valent antibody, antibody fragment, or antibody derivative thereof designed to at least partially target one or more microbial antigens associated with the cancer or cancer microenvironment. The treatment may comprise a targeted antibiotic against a particular kind of microbe or class of functionally or biologically similar microbes. In some cases, two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

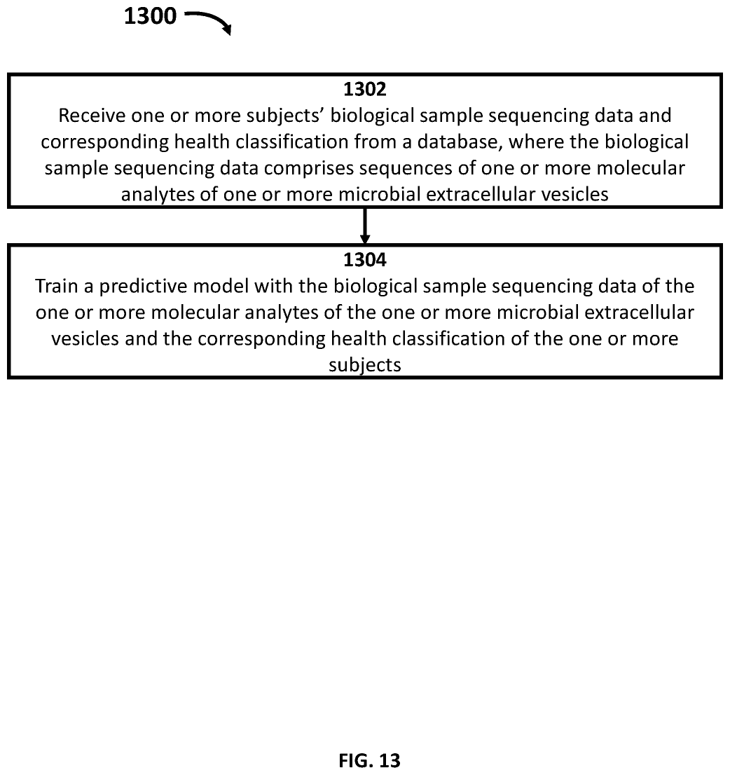

In some embodiments, the disclosure describes a computer-implemented method of training a predictive model 1300, as shown in FIG. 13. In some cases, the method comprises: (a) receiving one or more subjects' biological sample sequencing data and corresponding health classification from a database, where the biological sample sequencing data comprises sequences of one or more molecular analytes of one or more microbial extracellular vesicles 1302; and (b) training a predictive model with the biological sample sequencing data of the one or more molecular analytes of the one or more microbial extracellular vesicles and the corresponding health classification of the one or more subjects 1304. In some cases, the corresponding health classification may comprise cancerous, non-cancerous disease, non-cancerous non-disease (e.g., healthy). The biological sample may comprise a liquid biological sample, a tissue biological sample, or any combination thereof. The liquid biological sample may comprise plasma, serum, whole blood, urine, cerebral spinal fluid, saliva, sweat, tears, lymphatic fluid, exhaled breath condensate or any dilution, or processed fraction thereof. Whole blood may comprise plasma, white blood cells, red blood cells, platelets, or any combination thereof. In some instances, the tissue biological sample may comprise tissue homogenate.

In some cases, the one or more molecular analytes of the one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof.

In some cases, the method may further comprise decontaminating the one or more subjects' biological sample sequencing data, where decontaminating may be completed in-silico or using experimental controls.

In some cases, the method may further comprise aligning the one or more subjects' biological sample sequencing data to a human genome reference library and retaining the one or more sequencing reads that do not align to the human genome reference library thereby generating one or more microbial sequencing reads of the one or more molecular analytes of the one or more microbial extracellular vesicle. In some cases, the method may further comprise quantifying an abundance of the one or more molecular analytes of the one or more microbial extracellular vesicles

In some cases, the one or more subjects' biological sample sequencing data may be generated by whole genome sequencing, shotgun sequencing, next generation sequencing, targeted sequencing, RNA sequencing, methylation sequencing, or any combination thereof.

In some instances, the health classification of cancer may comprise a stage I or stage II cancer. The health classification of cancer may comprise bone, breast, lung, colon, brain, skin, ovary, pancreas, or any combination thereof type of cancers. The cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. The cancer may comprise one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some instances, the trained predictive model may be configured receive one or more subjects' biological sample sequencing data, one or more subjects' abundance of one or more molecular analytes of one or more microbial extracellular vesicles, or any combination thereof, as an input and output a predicted disease of the one or more subjects, a treatment for the disease of the one or more subjects, or any combination thereof. In some cases, the trained predictive model may be configured to predict one or more cancers, one or more subtypes of cancer, the anatomical location of one or more cancers, or any combination thereof one or more subjects. The trained predictive model may be configured to predict a stage of the cancer, prognosis of the cancer, mutation status of the cancer, future immunotherapy response of the cancer, an optimal therapy to treat the cancer, or any combination thereof. The trained predictive model may be configured to predict the cancer among one or more cancer types of the one or more subjects to identify a specific cancer type of the one or more cancer types. In some cases, the trained predictive model may comprise a machine learning model. The machine learning model may comprise one or more machine learning models, a regularized machine learning model, an ensemble of machine learning models, or any combination thereof. In some instances, the trained predictive model may comprise a random forest, neural network, naïve bayes, support vector machines, linear regression, k-nearest neighbors, k-means, decision tree, logistic regression, gradient boosting, or any combination thereof predictive model. In some cases, decontaminating the one or more subjects' biological sample sequencing data may improve an accuracy of the trained predictive model by at least 1%, at least 5%, at least 10%, at least 15%, or at least 20% when predicting the cancer of one or more subjects. In some instances, decontaminating the one or more subjects' biological sample sequencing data may increase an area under a receiver operating characteristic curve by at 1%, at least 2%, at least 4%, at least 5%, or at least 10%, when the trained predictive model when predicts the treatment for the disease of one or more subjects, or a disease of the one or more subjects.

In some cases, the treatment may comprise a repurposed treatment, which may or may not have been originally approved for targeting cancer. In some cases, the treatment may comprise a small molecule, a biologic, a probiotic, a virus, a bacteriophage, an immunotherapy, a broad-spectrum antibiotic, or any combination thereof. The probiotic may comprise an engineered bacterium strain or ensemble of engineered bacteria. In some cases, the treatment may comprise an adjuvant given in combination with a primary treatment against the subject's cancer to improve an efficacy of the primary treatment. In some cases, the treatment may comprise adoptive cell transfer to target microbial antigens associated with said cancer or cancer microenvironment. The treatment may comprise a cancer vaccine that exploits microbial antigens associated with the cancer or cancer microenvironment. The treatment may comprise a monoclonal antibody against microbial antigens associated with the cancer or cancer microenvironment. The treatment may comprise an antibody-drug conjugate designed to at least partially target microbial antigens associated with the cancer or cancer microenvironment. The treatment may comprise a multi-valent antibody, antibody fragment, or antibody derivative thereof designed to at least partially target one or more microbial antigens associated with the cancer or cancer microenvironment. The treatment may comprise a targeted antibiotic against a particular kind of microbe or class of functionally or biologically similar microbes. In some cases, two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

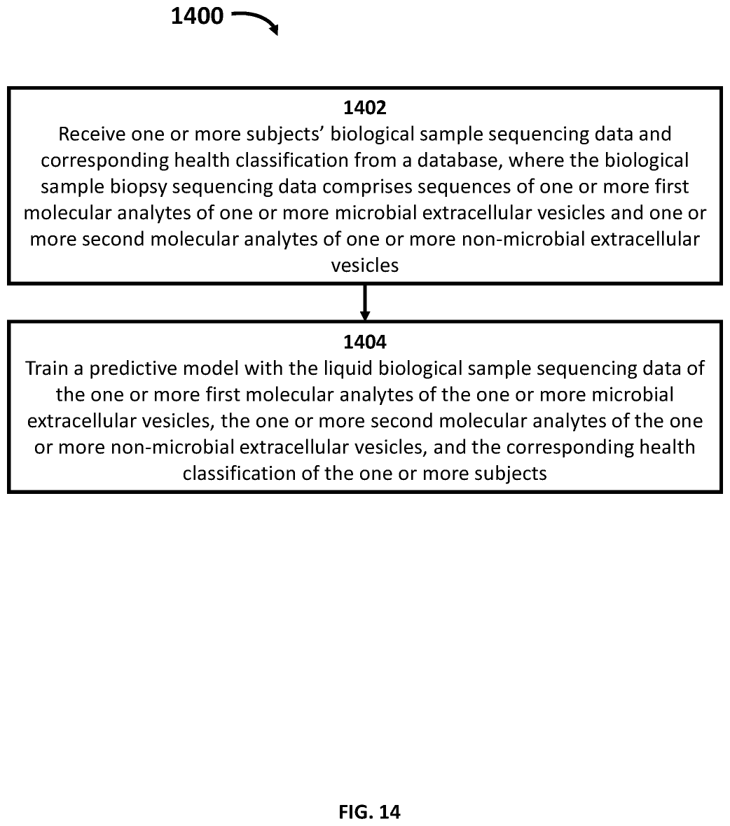

In some embodiments, the disclosure describes a computer-implemented method of training a predictive model 1400, as shown in FIG. 14. In some cases, the method comprises: (a) receiving one or more subjects' biological sample sequencing data and corresponding health classification from a database, where the biological sample sequencing data comprises sequences of one or more first molecular analytes of one or more microbial extracellular vesicles and one or more second molecular analytes of one or more non-microbial extracellular vesicles 1402; and (b) training a predictive model with the biological sample sequencing data of the one or more first molecular analytes of the one or more microbial extracellular vesicles, the one or more second molecular analytes of the one or more non-microbial extracellular vesicles, and the corresponding health classification of the one or more subjects 1404. In some cases, the corresponding health classification may comprise cancerous, non-cancerous disease, non-cancerous non-disease (e.g., healthy). The biological sample may comprise a liquid biological sample, a tissue biological sample, or any combination thereof. The liquid biological sample may comprise plasma, serum, whole blood, urine, cerebral spinal fluid, saliva, sweat, tears, lymphatic fluid, exhaled breath condensate or any dilution, or processed fraction thereof. Whole blood may comprise plasma, white blood cells, red blood cells, platelets, or any combination thereof. In some instances, the tissue biological sample may comprise tissue homogenate.

In some cases, the one or more molecular analytes of the one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. In some cases, the one or more molecular analytes of the one or more non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof.

In some cases, the method may further comprise decontaminating the one or more subjects' biological sample sequencing data, where decontaminating may be completed in-silico or using experimental controls.

In some cases, the method may further comprise quantifying an abundance of the one or more molecular analytes of the one or more microbial extracellular vesicles and/or an abundance of the one or more molecular analytes of the one or more non-microbial extracellular vesicles.

In some cases, the one or more subjects' biological sample sequencing data may be generated by whole genome sequencing, shotgun sequencing, next generation sequencing, targeted sequencing, RNA sequencing, methylation sequencing, or any combination thereof.

In some instances, the health classification of cancer may comprise a stage I or stage II cancer. The health classification of cancer may comprise bone, breast, lung, colon, brain, skin, ovary, pancreas, or any combination thereof type of cancers. The cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. The cancer may comprise one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some instances, the trained predictive model may be configured receive one or more subjects' biological sample sequencing data, one or more subjects' abundance of one or more molecular analytes of one or more microbial extracellular vesicles, one or more subjects' abundance of one or more molecular analytes of one or more non-microbial extracellular vesicles, or any combination thereof, as an input and output a predicted disease of the one or more subjects, a treatment for the disease of the one or more subjects, or any combination thereof. In some cases, the trained predictive model may be configured to predict one or more cancers, one or more subtypes of cancer, the anatomical location of one or more cancers, or any combination thereof one or more subjects. The trained predictive model may be configured to predict a stage of the cancer, prognosis of the cancer, mutation status of the cancer, future immunotherapy response of the cancer, an optimal therapy to treat the cancer, or any combination thereof. The trained predictive model may be configured to predict the cancer among one or more cancer types of the one or more subjects to identify a specific cancer type of the one or more cancer types. In some cases, the trained predictive model may comprise a machine learning model. The machine learning model may comprise one or more machine learning models, a regularized machine learning model, an ensemble of machine learning models, or any combination thereof. In some instances, the trained predictive model may comprise a random forest, neural network, naïve bayes, support vector machines, linear regression, k-nearest neighbors, k-means, decision tree, logistic regression, gradient boosting, or any combination thereof predictive model. In some cases, decontaminating the one or more subjects' biological sample sequencing data may improve an accuracy of the trained predictive model by at least 1%, at least 5%, at least 10%, at least 15%, or at least 20% when the trained predictive model is provided, as an input, a combined dataset of the abundance of one or more first molecular analytes of one or more microbial extracellular vesicles and the abundance of one or more second molecular analytes of one or more non-microbial extracellular vesicles.

In some instances, an area under a receiver operating characteristic curve for the predictive model when predicting a disease or a treatment for a disease may increase by at 1%, at least 2%, at least 4%, at least 5%, or at least 10%, when the trained predictive model is provided, as an input, a combined dataset of the abundance of one or more first molecular analytes of one or more microbial extracellular vesicles and the abundance of one or more second molecular analytes of one or more non-microbial extracellular vesicles.

In some cases, the treatment may comprise a repurposed treatment, which may or may not have been originally approved for targeting cancer. In some cases, the treatment may comprise a small molecule, a biologic, a probiotic, a virus, a bacteriophage, an immunotherapy, a broad-spectrum antibiotic, or any combination thereof. The probiotic may comprise an engineered bacterium strain or ensemble of engineered bacteria. In some cases, the treatment may comprise an adjuvant given in combination with a primary treatment against the subject's cancer to improve an efficacy of the primary treatment. In some cases, the treatment may comprise adoptive cell transfer to target microbial antigens associated with said cancer or cancer microenvironment. The treatment may comprise a cancer vaccine that exploits microbial antigens associated with the cancer or cancer microenvironment. The treatment may comprise a monoclonal antibody against microbial antigens associated with the cancer or cancer microenvironment. The treatment may comprise an antibody-drug conjugate designed to at least partially target microbial antigens associated with the cancer or cancer microenvironment. The treatment may comprise a multi-valent antibody, antibody fragment, or antibody derivative thereof designed to at least partially target one or more microbial antigens associated with the cancer or cancer microenvironment. The treatment may comprise a targeted antibiotic against a particular kind of microbe or class of functionally or biologically similar microbes. In some cases, two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

Although the above steps of the methods disclosed herein illustrate various embodiments of the disclosure, a person of ordinary skill in the art will recognize many variations based on the teaching described herein. In some cases, the steps may be completed in a different order. In some instances, steps may be added or omitted. In some embodiments, some of the steps may comprise sub-steps. Many of the steps may be repeated as often as beneficial.

### Predictive Models

The methods and systems of the present disclosure may utilize or access external capabilities of artificial intelligence, predictive models, and/or machine learning techniques to identify features of one or more analytes from MEV that may predict cancer. In some cases, the artificial intelligence techniques may identify features of one or more analytes from MEVs and one or more analytes of non-microbial EVs that may predict a cancer of one or more subjects. In some cases, the features may be used to train one or more predictive models, described elsewhere herein. These features may be used to accurately predict diseases or disorders. In some cases, the diseases or disorders may comprise cancer, as described elsewhere herein. Using such a predictive capability, health care providers (e.g., physicians) may be able to make informed, accurate risk-based decisions, thereby improving quality of care and monitoring provided to patients.

The methods and systems of the present disclosure may analyze the presence and abundance of a microbiome of sample through one or more analytes of MEVs, or one or more analytes of MEVs in combination with one or more non-microbial EVs to determine one or more microbial features and/or non-microbial features that may predict a disease of one or more subjects. In some cases, the methods, and systems, described elsewhere herein, may train a predictive model with the one or more microbial features and/or non-microbial features indicative of cancer of a subject. In some cases, the trained predictive model may then be used to generate a likelihood (e.g., a prediction) of cancer of one or more subjects that differ from the one or more subjects utilized to train the predictive model. The trained predictive model may comprise an artificial intelligence-based model, such as a machine learning based classifier, configured to process the one or more analytes of one or more MEVs, or the combined one or more analytes of one or more MEVs and one or more non-microbial EVs to generate the likelihood of the subject having the disease or disorder. The model may be trained using presence or abundance of the one or more analytes from one or more cohorts of patients, e.g., cancer patients, patients with non-cancerous diseases, patients with no disease and no cancer, cancer patients receiving a treatment for a cancer, patients receiving treatment for a non-cancerous disease, or any combination thereof. In some cases, the predictive model may be trained to provide a treatment prediction to treat a cancer of one or more patients that are not part of the training dataset of the predictive model. Such a predictive model may output a treatment recommendation for the one or more patients that are not part of the training dataset when provided an input of the patient's presence and abundance of one or more analytes from one or more MEVs, or a combined presence and abundance of one or more analytes from one or more MEVs and non-microbial EVs.

The model may comprise one or more predictive models. The model may comprise one or more machine learning algorithms. Examples of machine learning algorithms may include a support vector machine (SVM), a naïve Bayes classification, a random forest, a neural network (such as a deep neural network (DNN), a recurrent neural network (RNN), a deep RNN, a long short-term memory (LSTM) recurrent neural network (RNN), a gated recurrent unit (GRU), a gradient boosting machine, a random forest, or other supervised learning algorithm or unsupervised machine learning, statistical, linear regression, k-nearest neighbors, k-means, decision tree, logistic regression, or any combination thereof. The model may be used for classification or regression. The model may likewise involve the estimation of ensemble models, comprised of multiple predictive models, and utilize techniques such as gradient boosting, for example in the construction of gradient-boosting decision trees. The model may be trained using one or more training datasets corresponding to patient data e.g., patient medical history, family medical history, blood pressure, pulse, temperature, oxygen saturation or any combination thereof.

Training datasets may be generated from, for example, one or more cohorts of patients having common clinical disease or disorder diagnosis. Training datasets may comprise a set of MEVs, non-microbial EVs, or any combination thereof features in the form of presence and/or abundance of the one or more analytes of the MEVs or non-microbial EVs of a biological sample of one or more subjects. Features may comprise a corresponding cancer diagnosis of one or more subjects to aforementioned MEVs, non-microbial EVs, or any combination thereof features. In some cases, features may comprise patient information such as patient age, patient medical history, other medical conditions, current or past medications, clinical risk scores, and time since the last observation. For example, a set of features collected from a given patient at a given time point may collectively serve as a signature, which may be indicative of a health state or status of the patient at the given time point.

Labels may comprise clinical outcomes such as, for example, a presence, absence, diagnosis, or prognosis of a disease or disorder in the subject (e.g., patient). Clinical outcomes may comprise treatment efficacy (e.g., whether a subject is a positive responder to a cancer-based treatment).

Input features may be structured by aggregating the data into bins or alternatively using a one-hot encoding. Inputs may also include feature values or vectors derived from the previously mentioned inputs, such as cross-correlations.

Training records may be constructed from presence and/or abundance features of the one or more analytes of MEVs or a combination of the one or more analytes of MEVs and one or more analytes of non-microbial EVs.

The model may process the input features to generate output values comprising one or more classifications, one or more predictions, or a combination thereof. For example, such classifications or predictions may include a binary classification of a cancer or no cancer present in a subject (e.g., absence of a disease or disorder), a classification between a group of categorical labels (e.g., ‘no disease or disorder’, ‘apparent disease or disorder’, and ‘likely disease or disorder’), a likelihood (e.g., relative likelihood or probability) of developing a particular disease or disorder, a score indicative of a presence of disease or disorder, a ‘risk factor’ for the likelihood of mortality of the patient, and a confidence interval for any numeric predictions. Various machine learning techniques may be cascaded such that the output of a machine learning technique may also be used as input features to subsequent layers or subsections of the model.

In order to train the model (e.g., by determining weights and correlations of the model) to generate real-time classifications or predictions, the model can be trained using datasets, described elsewhere herein. Such datasets may be sufficiently large to generate statistically significant classifications or predictions. For example, datasets may comprise: databases of data including fungal and/or non-fungal microbial presence and/or abundance of one or more subjects' biological samples.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. In some embodiments, leave one out cross validation may be employed. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

To improve the accuracy of model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered or batch corrected to remove or mitigate confounding factors. For example, within a database, a subset of patients may be excluded.

The model may comprise one or more neural networks, such as a neural network, a convolutional neural network (CNN), a deep neural network (DNN), a recurrent neural network (RNN), or a deep RNN. The recurrent neural network may comprise units which can be long short-term memory (LSTM) units or gated recurrent units (GRU). For example, the model may comprise an algorithm architecture comprising a neural network with a set of input features such as vital sign and other measurements, patient medical history, and/or patient demographics. Neural network techniques, such as dropout or regularization, may be used during training the model to prevent overfitting. The neural network may comprise a plurality of sub-networks, each of which is configured to generate a classification or prediction of a different type of output information (e.g., which may be combined to form an overall output of the neural network). The machine learning model may alternatively utilize statistical or related algorithms including random forest, classification and regression trees, support vector machines, discriminant analyses, regression techniques, as well as ensemble and gradient-boosted variations thereof.

When the model generates a classification or a prediction of a disease or disorder, a notification (e.g., alert or alarm) may be generated and transmitted to a health care provider, such as a physician, nurse, or other member of the patient's treating team within a hospital. Notifications may be transmitted via an automated phone call, a short message service (SMS) or multimedia message service (MMS) message, an e-mail, or an alert within a dashboard. The notification may comprise output information such as a prediction of a disease or disorder, a likelihood of the predicted disease or disorder, a time until an expected onset of the disease or disorder, a confidence interval of the likelihood or time, or a recommended course of treatment for the disease or disorder.

To validate the performance of the model, different performance metrics may be generated. For example, an area under the receiver-operating characteristic curve (AUROC) may be used to determine the diagnostic capability of the model. For example, the model may use classification thresholds which are adjustable, such that specificity and sensitivity are tunable, and the receiver-operating characteristic curve (ROC) can be used to identify the different operating points corresponding to different values of specificity and sensitivity.

In some cases, such as when datasets are not sufficiently large, cross-validation may be performed to assess the robustness of a model across different training and testing datasets.

To calculate performance metrics such as sensitivity, specificity, accuracy, positive predictive value (PPV), negative predictive value (NPV), area under the precision-recall curve (AUPR), AUROC, or similar, the following definitions may be used. A “false positive” may refer to an outcome in which a positive outcome or result has been incorrectly or prematurely generated (e.g., before the actual onset of, or without any onset of, the disease or disorder). A “true positive” may refer to an outcome in which positive outcome or result has been correctly generated, when the patient has the disease or disorder (e.g., the patient shows symptoms of the disease or disorder, or the patient's record indicates the disease or disorder). A “false negative” may refer to an outcome in which a negative outcome or result has been generated, but the patient has the disease or disorder (e.g., the patient shows symptoms of the disease or disorder, or the patient's record indicates the disease or disorder). A “true negative” may refer to an outcome in which a negative outcome or result has been generated (e.g., before the actual onset of, or without any onset of, the disease or disorder).

The model may be trained until certain pre-determined conditions for accuracy or performance are satisfied, such as having minimum desired values corresponding to diagnostic accuracy measures. For example, the diagnostic accuracy measure may correspond to prediction of a likelihood of occurrence of a disease or disorder in the subject. As another example, the diagnostic accuracy measure may correspond to prediction of a likelihood of deterioration or recurrence of a disease or disorder for which the subject has previously been treated. Examples of diagnostic accuracy measures may include sensitivity, specificity, positive predictive value (PPV), negative predictive value (NPV), accuracy, AUPR, and AUROC corresponding to the diagnostic accuracy of detecting or predicting a disease or disorder.

For example, such a pre-determined condition may be that the sensitivity of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the disease or disorder comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPR) of predicting the disease or disorder comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the disease or disorder with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the disease or disorder with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the disease or disorder with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the disease or disorder with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the disease or disorder with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the disease or disorder with an area under the precision-recall curve (AUPR) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

The training data sets may be collected from training subjects (e.g., humans). Each training has a diagnostic status indicating that they have either been diagnosed with the biological condition or have not been diagnosed with the biological condition.

In some embodiments, the model is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, independent component analysis (ICA) is used to de-dimensionalize the data, such as that described in Lee, T.-W. (1998): Independent component analysis: Theory and applications, Boston, Mass: Kluwer Academic Publishers, ISBN 0-7923-8261-7, and Hyvarinen, A.; Karhunen, J.; Oja, E. (2001): Independent Component Analysis, New York: Wiley, ISBN 978-0-471-40540-5, which is hereby incorporated by reference in its entirety.

In some embodiments, principal component analysis (PCA) is used to de-dimensionalize the data, such as that described in Jolliffe, I. T. (2002). Principal Component Analysis. Springer Series in Statistics. New York: Springer-Verlag. doi:10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the model makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein; these gradient-boosting models are described in Boehmke, Bradley; Greenwell, Brandon (2019). “Gradient Boosting”. Hands-On Machine Learning with R. Chapman & Hall. pp. 221-245. ISBN 978-1-138-49568-5, which is hereby incorporated by reference in its entirety. In some embodiments, ensemble modeling techniques are used; these ensemble modeling techniques are described in the implementation of classification models herein, and are described in Zhou Zhihua (2012). Ensemble Methods: Foundations and Algorithms. Chapman and Hall/CRC. ISBN 978-1-439-83003-1, which is hereby incorporated by reference in its entirety. In some embodiments, the machine learning analysis is performed by a device executing one or more programs (e.g., one or more programs stored in the Non-Persistent Memory or in Persistent Memory) including instructions to perform the data analysis. In some embodiments, the data analysis is performed by a system comprising at least one processor (e.g., a processing core) and memory (e.g., one or more programs stored in Non-Persistent Memory or in the Persistent Memory) comprising instructions to perform the data analysis.

### Systems

The present disclosure provides computer systems that are programmed to implement methods of the disclosure. FIG. 15 shows a computer system 1501 that is programmed or otherwise configured to predict cancer, train a predictive model, generate a recommended therapeutic, or any combination thereof methods, described elsewhere herein. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1504 (e.g., random-access memory, read-only memory, flash memory), electronic storage unit 1506 (e.g., hard disk), communication interface 1508 (e.g., network adapter) for communicating with one or more other systems, and peripheral devices 1507, such as cache, other memory, data storage and/or electronic display adapters. The memory 1504, storage unit 1506, interface 1508 and peripheral devices 1507 are in communication with the CPU 1505 through a communication bus (solid lines), such as a motherboard. The storage unit 1506 can be a data storage unit (or data repository) for storing data. The computer system 1501 can be operatively coupled to a computer network (“network”) 1500 with the aid of the communication interface 1508. The network 1500 can be the Internet, an internet and/or extranet, or an intranet and/or extranet that is in communication with the Internet. The network 1500 in some cases is a telecommunication and/or data network. The network 1500 can include one or more computer servers, which can enable distributed computing, such as cloud computing. The network 1500, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1504. The instructions can be directed to the CPU 1505, which can subsequently program or otherwise configure the CPU 1505 to implement methods of the present disclosure, described elsewhere herein. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1506 can store files, such as drivers, libraries, and saved programs. The storage unit 1506 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1500. For instance, the computer system 1501 can communicate with a remote computer system of a user. Examples of remote computer systems may include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1500.

Methods as described herein can be implemented by way of machine (e.g., computer processor) executable code stored on an electronic storage location of the computer system 1501, such as, for example, on the memory 1504 or electronic storage unit 1506. The machine executable or machine-readable code can be provided in the form of software. During use, the code can be executed by the processor 1505. In some cases, the code can be retrieved from the storage unit 1506 and stored on the memory 1504 for ready access by the processor 1505. In some situations, the electronic storage unit 1506 can be precluded, and machine-executable instructions are stored on memory 1504.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1502 that comprises a user interface (UI) 1503 for providing, for example, a display for visualization of prediction results or an interface for training a predictive model. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, predict cancer of a subject or subjects, determine a tailored treatment and/or therapeutic to treat a subject's or subjects' cancer, or any combination thereof.

In some cases, the computer system may comprise a computer system configured to identify a disease of a subject. In some instances, the computer system, may comprise: (a) one or more processors; and (b) a non-transient computer readable storage medium including software, where the software comprises executable instructions that, as a result of execution, cause the one or more processors of the computer system to: (i) receive a liquid biological sample of a subject comprising one or more microbial extracellular vesicles; (ii) enrich the one or more microbial extracellular vesicles with one or more affinity reagents; (iii) detect one or more molecular analytes from the one or more microbial extracellular vesicles; and (iv) identify the disease of the subject based on the one or more molecular analytes obtained from the one or more microbial extracellular vesicles. In some cases, the liquid biological sample may further comprise one or more non-microbial extracellular vesicles. In some cases, the executable instructions may further comprise quantifying an abundance of the one or more molecular analytes. In some instances, the one or more molecular analytes comprise vesicle-associated cell-free microbial DNA, microbial RNA, non-microbial DNA, non-microbial RNA, microbial proteins, non-microbial proteins, microbial metabolites, non-microbial metabolites, microbial lipids, non-microbial lipids, microbial glycans, non-microbial glycans, or any combination thereof. In some cases, the subject may comprise a non-human mammal or a human subject.

In some cases, the liquid biological sample may comprise plasma, serum, whole blood, urine, cerebral spinal fluid, saliva, sweat, tears, lymphatic fluid, exhaled breath condensate, or any dilution, or processed fraction thereof. In some instances, whole blood comprises plasma, white blood cells, red blood cells, platelets, or any combination thereof.

In some cases, the one or more affinity reagents are configured to couple to one or more microbial or one or more non-microbial cell wall molecular motifs. The one or more microbial cell wall molecular motifs or the one or more non-microbial cell wall molecular motifs may comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combination thereof. In some instances, the one or more affinity reagents may comprise recombinant innate immunity patterns recognition receptors or one or more antibodies, where the one or more antibodies may comprise polyclonal antibodies, monoclonal antibodies, recombinant antibodies, aptamers, single-chain variable fragment (scFv), single-chain antibodies, or any combination thereof. In some cases, the recombinant innate immunity pattern recognition receptors may comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. The derivative thereof may comprise an epitope tag and full-length recombinant innate immunity pattern recognition receptors or recombinant soluble ectodomains thereof. The epitope tag may comprise a N- or C-terminal 6× histidine tag, green fluorescent protein (GFP), myc, hemagglutinin (HA), Fc fusion, biotin or any combination thereof.

In some cases, the one or more antibodies, aptamers, single-chain variable fragments (scFv), or said single-chain antibodies may comprise an epitope tag. In some cases, the epitope tag may comprise a N- or C-terminal 6×-histidine tag, green fluorescent protein (GFP), myc, hemagglutinin (HA), Fc fusion, biotin, or any combination thereof.

In some instances, the one or more affinity reagents may comprise a region to interact with the one or more microbial cell wall molecular motifs or the one or more non-microbial cell wall molecular motifs. In some cases, the one or more affinity reagents may comprise an epitope tag.

In some cases, enrich the one or more microbial extracellular vesicles may comprise contacting the liquid biological sample with a support comprising covalently immobilized affinity agents. In some instances, the covalently immobilized affinity agents may comprise a region that interacts with the one or more microbial cell wall molecular motifs or the one or more non-microbial cell wall molecular motifs. In some cases, the support may comprise a magnetic bead, an agarose bead, non-magnetic latex, functionalized Sepharose, pH-sensitive polymers or any combinations thereof. In some instances, the support may comprise an epitope tag recognition surface. In some instances, the epitope tag recognition surface may comprise streptavidin, antibodies specific for 6×-histidine tag, GFP, myc, HA, biotin, or any combination thereof. IN some cases, the epitope tag recognition surface may comprise an anti-species antibody.

In some cases, enrich may comprise: (a) contact the liquid biological sample with the one or more affinity reagents to form capture reagent-molecular motif interaction complexes; (b) contact the capture reagent-molecular motif interaction complexes with a support; and (c) separate the support from the liquid biological sample to concentrate the capture reagent-molecular motif complexes.

In some instances, detect the one or more analytes may comprise nucleic acid sequencing, polymerase chain reaction (PCR), or hybridization-based analysis for nucleic acid analytes. In some cases, nucleic acid sequencing may comprise whole genome sequencing, shotgun sequencing, next generation sequencing, targeted sequencing, RNA sequencing, methylation sequencing, or any combination thereof. In some cases, detect the one or more analytes may comprise performing mass spectrometry analysis, liquid chromatography-mass spectrometry (LC-MS), or high-performance liquid chromatography (HPLC). In some cases, detect the one or more analytes may comprise performing immunoassay analysis.

In some instances, the disease may comprise cancer. The cancer may comprise a stage I or stage II cancer. In some instances, the cancer may comprise bone, breast, lung, colon, brain, skin, ovary, pancreas, or any combination thereof type of cancer. In some instances, the cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprise a one or more cancer types outside a subject's intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, identify the disease of the subject based on the one or more molecular analytes may comprise predicting the cancer of a subject by a predictive model, where the predictive model may be trained with one or more subjects' one or more molecular analytes obtained from one or more microbial extracellular vesicles and an associated disease of the one or more subjects. In some cases, the associated disease of the one or more subjects may comprise cancer, as described elsewhere herein. In some instances, the predictive model may be configured to receive the one or more molecular analytes of the subject as an input and output the disease of the subject. In some cases, the predictive model may be configured to predict one or more cancers, one or more subtypes of cancer, the anatomical locations of one or more cancers, or any combination thereof of said subject. In some cases, the predictive model is configured to predict a stage of the cancer, prognosis of the cancer, mutation status of the cancer, future immunotherapy response of the cancer, an optimal therapy to treat the cancer, or any combination thereof. In some instances, the predictive model may be configured to predict the cancer among one or more cancer types of the subject to identify a specific cancer type of the one or more cancer types.

In some instances, the predictive model may comprise a machine learning model, described elsewhere herein, where the machine learning model may comprise a regularized machine learning model, ensemble of machine learning models, or any combination thereof. In some cases, the predictive model may comprise a random forest, neural network, naïve bayes, support vector machines, learning regression, k-nearest neighbors, k-means, decision tree, logistic regression, gradient boosting, or any combination thereof.

In some cases, enrich the one or more microbial extracellular vesicles with one or more affinity reagents may improve an accuracy of the predictive model by at least 1%, at least 5%, at least 10%, at least 15%, or at least 20% when predicting the cancer of the subject.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “microbial extracellular vesicle” (“MEV”) generally describes non-replicative, membrane enclosed structures derived from parent microbial cells. MEVs may contain molecular constituents derived from a parent cell; in the case of microbial colonization of non-microbial cells or tissues, MEVs may also contain molecular constituents derived from their host cell or host tissue. “Microbial extracellular vesicle” (“MEV”) may also generally describe any non-replicative, microbially derived structure, such as soluble or insoluble aggregates, comprising molecular features (e.g., lipopolysaccharide, microbial glycoproteins, or lipids). MEVs can be affinity-enriched by the methods of the present invention.

The terms “microbes” or “microbial” are used interchangeably herein to refer to types of microorganisms. The microorganisms may comprise, e.g., viruses, bacteria, fungi, archaea, or any combination thereof. Accordingly, a “microbe” may comprise a single microorganism of a plurality of microbes.

The term “non-microbial extracellular vesicle” (“non-microbial EV” or “non-microbial EVs”) generally describes non-replicative, lipid bilayer membrane enclosed structures derived from parent subject cells. Non-microbial EVs may be about 30 to 2,000 nm in diameter. Non-microbial EVs may contain molecular constituents derived from a parent subject cell. Non-microbial EVs may also contain molecular constituents derived from microbial sources (for example, in the case of tissue colonization by microbes).

The term “extracellular vesicle” (“EV”) generally describes non-replicative membrane enclosed structures. EVs may be derived from microbial and/or non-microbial parent cells.

The term “in vivo” generally describes an event that takes place in a subject's body.

The term “ex vivo” generally describes an event that takes place outside of a subject's body. An ex vivo assay is generally not performed on a subject. Rather, it may be performed upon a sample separate from a subject. An example of an ex vivo assay performed on a sample may be an “in vitro” assay.

The term “in vitro” generally describes an event that takes places outside of subject's biological context. In some cases, the context may be contained area in a laboratory, such that the material being studied may be separated from the biological source. In some cases, the contained area may be a fume hood, glove box, Petri dish, test tubes, flasks, etc. In vitro assays can encompass cell-based assays in which living or dead cells may be employed. In vitro assays can also encompass a cell-free assay in which no intact cells may be employed.

As used herein, the term “about” a number refers to that number plus or minus 10% of that number. The term “about” a range refers to that range minus 10% of its lowest value and plus 10% of its greatest value.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Embodiments

Numbered embodiment 1 comprises a method of identifying a disease of a subject, comprising: providing a biological sample from a subject comprising one or more microbial extracellular vesicles; enriching said one or more microbial extracellular vesicles with one or more affinity reagents; detecting one or more molecular analytes from said one or more microbial extracellular vesicles; and identifying said disease of said subject based on said one or more molecular analytes from said one or more microbial extracellular vesicles. Numbered embodiment 2 comprises the method of embodiment 1, wherein said biological sample comprises non-microbial extracellular vesicles. Numbered embodiment 3 comprises the method as in embodiments 1 or 2, further comprising quantifying an abundance of said one or more molecular analytes. Numbered embodiment 4 comprises the method as in any of embodiments 1-3, wherein said one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. Numbered embodiment 5 comprises the method as in any of embodiments 1-4, wherein said one or more molecular analytes of said one or more non-microbial extracellular comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. Numbered embodiment 6 comprises the method as in any of embodiments 1-5, wherein said biological sample comprises a liquid biological sample, tissue biological sample, or any combination thereof. Numbered embodiment 7 comprises the method as in any of embodiments 1-6, wherein said liquid biological sample comprises plasma, serum, whole blood, urine, cerebral spinal fluid, saliva, sweat, tears, lymphatic fluid, exhaled breath condensate or any dilution, or processed fraction thereof. Numbered embodiment 8 comprises the method as in any of embodiments 1-7, wherein said whole blood comprises plasma, white blood cells, red blood cells, platelets, or any combination thereof. Numbered embodiment 9 comprises the method as in any of embodiments 1-6, wherein said tissue biological sample comprises tissue homogenate. Numbered embodiment 10 comprises the method as in any of embodiments 1-9, wherein said one or more affinity reagents are configured to couple to one or more microbial or one or more non-microbial cell wall molecular motifs. Numbered embodiment 11 comprises the method as in any of embodiments 1-10, wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof. Numbered embodiment 12 comprises the method as in any of embodiments 1-11, wherein said one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. Numbered embodiment 13 comprises the method as in any of embodiments 1-12, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. Numbered embodiment 14 comprises the method as in any of embodiments 1-13, wherein said derivatives thereof comprise an epitope tag and full-length recombinant innate immunity pattern recognition receptors or recombinant soluble ectodomains thereof. Numbered embodiment 15 comprises the method as in any of embodiments 1-14, wherein said epitope tag comprises a N- or C-terminal 6×-histidine tag, green fluorescent protein (GFP), myc, hemagglutinin (HA), Fc fusion, biotin or any combination thereof. Numbered embodiment 16 comprises the method as in any of embodiments 1-12, wherein said one or more antibodies, aptamers, single-chain variable fragments (scFv), or said single-chain antibodies comprise an epitope tag. Numbered embodiment 17 comprises the method as in embodiments 1-12, or 16, wherein said epitope tag comprises a N- or C-terminal 6×-histidine tag, green fluorescent protein (GFP), myc, hemagglutinin (HA), Fc fusion, biotin or any combination thereof. Numbered embodiment 18 comprises the method as in any of embodiments 1-17, wherein said one or more affinity reagents comprise a region to interact with said one or more microbial cell wall molecular motifs or said one or more non-microbial cell wall molecular motifs. Numbered embodiment 19 comprises the method as in any of embodiments 1-18, wherein said enriching comprises contacting said biological sample with a support comprising covalently immobilized affinity agents. Numbered embodiment 20 comprises the method as in any of embodiments 1-19, wherein said covalently immobilized affinity agents comprise a region that interacts with said one or more microbial cell wall molecular motifs or said one or more non-microbial cell wall molecular motifs. Numbered embodiment 21 comprises the method as in any of embodiments 1-20, wherein said supports comprise a magnetic bead, an agarose bead, non-magnetic latex, functionalized Sepharose, pH-sensitive polymers or any combination thereof.

Numbered embodiment 22 comprises the method as in any of embodiment 1-21, wherein said enriching comprises:
contacting said biological sample with said one or more affinity reagents to form capture reagent-molecular motif interaction complexes; contacting said capture reagent-molecular motif interaction complexes with a support; and separating said support from said biological sample to concentrate said capture reagent-molecular motif interaction complexes. Numbered embodiment 23 comprises the method as in any of embodiments 1-22, wherein said one or more affinity reagents comprise an epitope tag. Numbered embodiment 24 comprises the method as in any of embodiments 1-23, wherein said support comprises an epitope tag recognition surface. Numbered embodiment 25 comprises the method as in any of embodiments 1-24, wherein said epitope tag recognition surface comprises streptavidin, antibodies specific for 6×-histidine tag, green fluorescent protein (GFP), myc, hemagglutinin (HA), biotin, or any combination thereof. Numbered embodiment 26 comprises the method as in any of embodiments 1-25, wherein said epitope tag recognition surface comprises an anti-species antibody. Numbered embodiment 27 comprises the method as in any of embodiments 1-26, wherein said detecting comprises nucleic acid sequencing, polymerase chain reaction (PCR), or hybridization-based analysis of the one or more molecular analytes. Numbered embodiment 28 comprises the method as in any of embodiments 1-27, wherein said nucleic acid sequencing comprises whole genome sequencing, shotgun sequencing, next generations sequencing, targeted sequencing, RNA sequencing, methylation sequencing, or any combination thereof. Numbered embodiment 29 comprises the method as in any of embodiments 1-28, wherein said detecting comprises performing mass spectrometry analyses, liquid chromatography-mass spectrometry (LC-MS), or high-performance liquid chromatography (HPLC). Numbered embodiment 30 comprises the method as in any of embodiments 1-29, wherein said detecting comprises performing immunoassay analysis. Numbered embodiment 31 comprises the method as in any of embodiments 1-30, wherein said disease comprises cancer. Numbered embodiment 32 comprises the method as in any of embodiments 1-31, wherein said cancer comprises a stage I or stage II cancer. Numbered embodiment 33 comprises the method as in any of embodiments 1-32, wherein said cancer comprises bone, breast, lung, colon, brain, skin, ovary, pancreas, or any combination thereof type of cancer. Numbered embodiment 34 comprises the method as in any of embodiments 1-33, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 35 comprises the method as in any of embodiments 1-34, wherein said cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 36 comprises the method as in any of embodiments 1-35, wherein said identifying said cancer comprises predicting said cancer by a predictive model, wherein said predictive model is trained with one or more subjects' one or more molecular analytes obtained from one or more microbial extracellular vesicles and an associated disease of said one more subjects. Numbered embodiment 37 comprises the method as in any of embodiments 1-36, wherein said predictive model is configured to receive said one or more molecular analytes of said subject as an input, and output said disease of said subject. Numbered embodiment 38 comprises the method as in any of embodiments 1-37, wherein said predictive model is configured to predict one or more cancers, one or more subtypes of cancer, the anatomical locations of one or more cancers, or any combination thereof of said subject. Numbered embodiment 39 comprises the method as in any of embodiments 1-38, wherein said predictive model is configured to predict a stage of said cancer, prognosis of said cancer, mutation status of said cancer, future immunotherapy response of said cancer, an optimal therapy to treat said cancer, or any combination thereof. Numbered embodiment 40 comprises the method as in any of embodiments 1-39, wherein said predictive model is configured to predict said cancer among one or more cancer types of said subject to identify a specific cancer type of said one or more cancer types. Numbered embodiment 41 comprises the method as in any of embodiments 1-40, wherein said predictive model comprises a machine learning model, wherein said machine learning model comprises a regularized machine learning model, ensemble of machine learning models, or any combination thereof. Numbered embodiment 42 comprises the method as in any of embodiments 1-41, wherein said predictive model comprises a random forest, neural network, naïve bayes, support vector machines, learning regression, k-nearest neighbors, k-means, decision tree, logistic regression, gradient boosting, or any combination thereof. Numbered embodiment 43 comprises the method as in any of embodiments 1-42, wherein enriching said microbial extracellular vesicles improves an accuracy of said predictive model by at least 1%, at least 5%, at least 10%, at least 15%, or at least 20% when predicting said cancer of said subject. Numbered embodiment 44 comprises the method as in any of embodiments 1-43, wherein said subject comprises a non-human mammal or a human subject.

Numbered embodiment 45 comprises a method of identifying a disease of a subject comprising: providing biological sample of a subject comprising one or more microbial extracellular vesicles and one or more non-microbial extracellular vesicles; enriching said one or more microbial extracellular vesicles with one or more first affinity reagents and said one or more non-microbial extracellular vesicles with one or more second affinity reagents, thereby generating one or more enriched microbial extracellular vesicles and one or more enriched non-microbial extracellular vesicles; detecting a first abundance of one or more molecular analytes from said enriched one or more microbial extracellular vesicles and a second abundance of one or more molecular analytes from said enriched one or more non-microbial extracellular vesicles; and identifying said disease of said subject from an association between a combination of said first abundance of one or more molecular analytes and said second abundances of one or more molecular analytes, and a model of diseases associated with a third abundance of one or more molecular analytes from one or more microbial extracellular vesicles and a fourth abundance of one or more molecular analytes from one or more non-microbial extracellular vesicles. Numbered embodiment 46 comprises the method of embodiment 45, wherein detecting comprises quantifying said first abundance of one or more molecular analytes and said second abundance of one or more molecular analytes. Numbered embodiment 47 comprises the method as in embodiments 45 or 46, wherein said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. Numbered embodiment 48 comprises the method as in any of embodiments 45-47, wherein said one or more molecular analytes of said one or more non-microbial extracellular vesicles comprises cell-free non-microbial DNA, cell-free non-microbial RNA, non-microbial DNA, non-microbial RNA, non-microbial proteins, non-microbial metabolites, non-microbial lipids, non-microbial glycans, or any combination thereof. Numbered embodiment 49 comprises the method as in any of embodiments 45-48, wherein said biological sample comprises a liquid biological sample, tissue biological sample, or any combination thereof. Numbered embodiment 50 comprises the method as in any of embodiments 45-49, wherein said liquid biological sample comprises plasma, serum, whole blood, urine, cerebral spinal fluid, saliva, sweat, tears, lymphatic fluid, exhaled breath condensate, or any combination, any dilution, or processed fraction thereof. Numbered embodiment 51 comprises the method as in any of embodiments 45-50, wherein said whole blood comprises white blood cells, plasma, red blood cells, platelets, or any combination thereof. Numbered embodiment 52 comprises the method as in any of embodiments 45-49, wherein said tissue biological sample comprises tissue homogenate. Numbered embodiment 53 comprises the method as in any of embodiments 45-52, wherein enriching comprises concentrating one or more microbial cell wall molecular motifs or one or more non-microbial cell wall molecular motifs. Numbered embodiment 54 comprises the method as in any of embodiments 45-53 wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof. Numbered embodiment 55 comprises the method as in any of embodiments 45-54, wherein said one or more first affinity reagents or said one or more second affinity reagents comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof. Numbered embodiment 56 comprises the method as in any of embodiments 45-55, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof. Numbered embodiment 57 comprises the method as in any of embodiments 45-56, wherein said derivatives thereof comprise an epitope tag, full-length recombinant innate immunity pattern recognition receptors, or recombinant soluble ectodomains thereof. Numbered embodiment 58 comprises the method as in any of embodiments 45-57, wherein said one or more antibodies, said aptamers, said single-chain variable fragments (scFv), and said single-chain antibodies comprise an epitope tag. Numbered embodiment 59 comprises the method as in any of embodiments 45-58, wherein said epitope tag comprises a N- or C-terminal 6×-histidine tag, green fluorescent protein (GFP), myc, hemagglutinin (HA), Fc fusion, biotin or any combination thereof. Numbered embodiment 60 comprises the method as in any of embodiments 45-59, wherein said one or more first affinity reagents and said one or more second affinity reagents comprise a region to interact with said one or more microbial cell wall molecular motifs or said one or more non-microbial cell wall molecular motifs. Numbered embodiment 61 comprises the method as in any of embodiments 45-60, wherein enriching comprises incubating said biological sample with a support comprising covalently immobilized affinity agents. Numbered embodiment 62 comprises the method as in any of embodiments 45-61, wherein said covalently immobilized affinity agents comprise a region that interacts with said one or more microbial cell wall molecular motifs or said one or more non-microbial cell wall molecular motifs. Numbered embodiment 63 comprises the method as in any of embodiments 45-62, wherein said supports comprise a magnetic bead, an agarose bead, non-magnetic latex, functionalized Sepharose, pH-sensitive polymers or any combinations thereof. Numbered embodiment 64 comprises the method as in any of embodiments 45-63, wherein said one or more first affinity reagents or said one or more second affinity reagents are specific to mammalian antigens CD9, CD63, CD81, glypican 1 (GPC1), Mart-1, TYRP2, Human epidermal growth factor receptors (HER) family members, EpCAM, or any combination thereof. Numbered embodiment 65 comprises the method as in any of embodiments 45-64, wherein detecting comprises performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with said one or more molecular analytes of said one or more microbial extracellular vesicles or said one or more molecular analytes from said one or more non-microbial extracellular vesicles. Numbered embodiment 66 comprises the method as in any of embodiments 45-65, wherein nucleic acid sequencing comprises whole genome sequencing, shotgun sequencing, next generations sequencing, targeted sequencing, RNA sequencing, methylation sequencing, or any combination thereof. Numbered embodiment 67 comprises the method as in any of embodiments 45-66, wherein said model comprises a predictive model, wherein the predictive model is trained with one or more subjects' said third abundance of one or more molecular analytes from said one or more microbial extracellular vesicles, said fourth abundance of one or more molecular analytes from said one or more non-microbial extracellular vesicles, and a corresponding disease of said one or more subjects. Numbered embodiment 68 comprises the method as in any of embodiments 45-67, wherein said predictive model is configured to receive said first abundance of one or more molecular analytes and said second abundance of one or more molecular analytes as an input and output a prediction of said disease of said subject. Numbered embodiment 69 comprises the method as in any of embodiments 45-68, wherein said disease comprises cancer. Numbered embodiment 70 comprises the method as in any of embodiments 45-69, wherein said cancer comprises a stage I or stage II cancer. Numbered embodiment 71 comprises the method as in any of embodiments 45-70, wherein said cancer comprises bone, breast, lung, colon, brain, skin, ovary, pancreas, or any combination thereof type of cancer. Numbered embodiment 72 comprises the method as in any of embodiments 45-71, wherein said predictive model is configured to predict one or more cancers, one or more subtypes of cancer, anatomical locations of one or more cancers, or any combination thereof in said subject. Numbered embodiment 73 comprises the method as in any of embodiments 45-72, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 74 comprises the method as in any of embodiments 45-73, wherein said cancer comprises one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 75 comprises the method as in any of embodiments 45-74, wherein said predictive model comprises a machine learning model. Numbered embodiment 76 comprises the method as in any of embodiments 45-75, wherein said machine learning model comprises one or more machine learning models, a regularized machine learning model, an ensemble of machine learning models, or any combination thereof. Numbered embodiment 77 comprises the method as in any of embodiments 45-76, wherein said predictive model comprises a random forest, neural network, naïve bayes, support vector machines, linear regression, k-nearest neighbors, k-means, decision tree, logistic regression, gradient boosting, or any combination thereof predictive model. Numbered embodiment 78 comprises the method as in any of embodiments 45-77, wherein said subject comprises a human or a non-human mammal. Numbered embodiment 79 comprises the method as in any of embodiments 45-78, wherein enriching said one or more microbial extracellular vesicles and said one or more non-microbial extracellular vesicles improves an accuracy of said predictive model by at least 1%, at least 5%, at least 10%, at least 15%, or at least 20% when predicting said cancer of said subject. Numbered embodiment 80 comprises the method as in any of embodiments 45-79, wherein an area under a receiver operating characteristic curve of said predictive model when predicting said disease of said subject is increase by at least 1%, at least 2%, at least 4%, at least 5%, or at least 10%, when said combination of said first abundance of one or more molecular analytes and said second abundance of one or more molecular analytes are provided as said input to said predictive model.

Numbered embodiment 81 comprises a method of identifying a treatment for a disease of a subject, comprising: providing a biological sample of a subject comprising one or more microbial extracellular vesicles; enriching said one or more microbial extracellular vesicles with one or more affinity reagents, thereby generating one or more enriched microbial extracellular vesicles; detecting one or more molecular analytes from said one or more enriched microbial extracellular vesicles; and identifying said treatment for said disease of said subject based on said one or more molecular analytes. Numbered embodiment 82 comprises the method of embodiment 81, wherein said biological sample comprises non-microbial extracellular vesicles and said one or more microbial extracellular vesicles. Numbered embodiment 83 comprises the method as in embodiments 81 or 82, further comprising quantifying an abundance of said one or more molecular analytes. Numbered embodiment 84 comprises the method as in any of embodiments 81-83, wherein said one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. Numbered embodiment 85 comprises the method as in any of embodiments 81-84, wherein said one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. Numbered embodiment 86 comprises the method as in any of embodiments 81-85, wherein said biological sample comprises a liquid biological sample, tissue biological sample, or any combination thereof. Numbered embodiment 87 comprises the method as in any of embodiments 81-86, wherein said liquid biological sample comprises plasma, serum, whole blood, urine, cerebral spinal fluid, saliva, sweat, tears, lymphatic fluid, exhaled breath condensate or any dilution, or processed fraction thereof. Numbered embodiment 88 comprises the method as in any of embodiments 81-87, wherein said whole blood comprises plasma, white blood cells, red blood cells, platelets, or any combination thereof. Numbered embodiment 89 comprises the method as in any of embodiments 81-86, wherein said tissue biological sample comprises tissue homogenate. Numbered embodiment 90 comprises the method as in any of embodiments 81-89, wherein said one or more affinity reagents are configured to couple to one or more microbial or one or more non-microbial cell wall molecular motifs. Numbered embodiment 91 comprises the method as in any of embodiments 81-90, wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof. Numbered embodiment 92 comprises the method as in any of embodiments 81-91, wherein said one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. Numbered embodiment 93 comprises the method as in any of embodiments 81-92, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. Numbered embodiment 94 comprises the method as in any of embodiments 81-93, wherein said derivatives thereof comprise an epitope tag and full-length recombinant innate immunity pattern recognition receptors or recombinant soluble ectodomains thereof. Numbered embodiment 95 comprises the method as in any of embodiments 81-94, wherein said epitope tag comprises a N- or C-terminal 6×-histidine tag, green fluorescent protein (GFP), myc, hemagglutinin (HA), Fc fusion, biotin, or any combination thereof. Numbered embodiment 96 comprises the method as in any of embodiments 81-92, wherein said one or more antibodies, aptamers, single-chain variable fragments (scFv), or said single-chain antibodies comprise an epitope tag. Numbered embodiment 97 comprises the method as in embodiments 81-92, or 96, wherein said epitope tag comprises a N- or C-terminal 6×-histidine tag, green fluorescent protein (GFP), myc, hemagglutinin (HA), Fc fusion, biotin or any combination thereof. Numbered embodiment 98 comprises the method as in any of embodiments 81-97, wherein said one or more affinity reagents comprise a region to interact with said one or more microbial cell wall molecular motifs or said one or more non-microbial cell wall molecular motifs. Numbered embodiment 99 comprises the method as in any of embodiments 81-98, wherein said enriching comprises contacting said biological sample with a support comprising covalently immobilized affinity agents. Numbered embodiment 100 comprises the method as in any of embodiments 81-99, wherein said covalently immobilized affinity agents comprise a region that interacts with said one or more microbial cell wall molecular motifs or said one or more non-microbial cell wall molecular motifs. Numbered embodiment 101 comprises the method as in any of embodiments 81-100, wherein said supports comprise a magnetic bead, an agarose bead, non-magnetic latex, functionalized Sepharose, pH-sensitive polymers or any combinations thereof.

Numbered embodiment 102 comprises a method as in any of embodiment 81-101, wherein said enriching comprises: contacting said biological sample with said one or more affinity reagents to form capture reagent-molecular motif interaction complexes; contacting said capture reagent-molecular motif interaction complexes with a support; and separating said support from said biological sample to concentrate said capture reagent-molecular motif interaction complexes. Numbered embodiment 103 comprises the method as in any of embodiments 81-102, wherein said one or more affinity reagents comprise an epitope tag. Numbered embodiment 104 comprises the method as in any of embodiments 81-103, wherein said support comprises an epitope tag recognition surface. Numbered embodiment 105 comprises the method as in any of embodiments 81-104, wherein said epitope tag recognition surface comprises streptavidin, antibodies specific for 6×-histidine tag, green fluorescent protein (GFP), myc, hemagglutinin (HA), biotin, or any combination thereof. Numbered embodiment 106 comprises the method as in any of embodiments 81-105, wherein said epitope tag recognition surface comprises an anti-species antibody. Numbered embodiment 107 comprises the method as in any of embodiments 81-106, wherein said detecting comprises nucleic acid sequencing, polymerase chain reaction (PCR)-based, or hybridization-based analysis for nucleic acid analytes. Numbered embodiment 108 comprises the method as in any of embodiments 81-107, wherein said nucleic acid sequencing comprises whole genome sequencing, shotgun sequencing, next generations sequencing, targeted sequencing, RNA sequencing, methylation sequencing, or any combination thereof. Numbered embodiment 109 comprises the method as in any of embodiments 81-108, wherein said detecting comprises performing mass spectrometry analyses, liquid chromatography-mass spectrometry (LC-MS), or high-performance liquid chromatography (HPLC). Numbered embodiment 110 comprises the method as in any of embodiments 81-109, wherein said detecting comprises performing immunoassay analysis. Numbered embodiment 111 comprises the method as in any of embodiments 81-110, wherein said disease comprises cancer. Numbered embodiment 112 comprises the method as in any of embodiments 81-111, wherein said cancer comprises a stage I or stage II cancer. Numbered embodiment 113 comprises the method as in any of embodiments 81-112, wherein said cancer comprises bone, breast, lung, colon, brain, skin, ovary, pancreas, or any combination thereof type of cancer. Numbered embodiment 114 comprises the method as in any of embodiments 81-113, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 115 comprises the method as in any of embodiments 81-114, wherein said cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 116 comprises the method as in any of embodiments 81-115, wherein said identifying said treatment for said disease comprises predicting said treatment by a predictive model, wherein said predictive model is trained with one or more subjects' one or more molecular analytes obtained from one or more microbial extracellular vesicles and an associated treatment for said disease of said one more subjects. Numbered embodiment 117 comprises the method as in any of embodiments 81-116, wherein said predictive model is configured to receive said one or more molecular analytes of said subject an input, and output said treatment for said disease of said subject. Numbered embodiment 118 comprises the method as in any of embodiments 81-117, wherein said predictive model comprises a machine learning model, wherein said machine learning model comprises a regularized machine learning model, ensemble of machine learning models, or any combination thereof. Numbered embodiment 119 comprises the method as in any of embodiments 81-118, wherein said predictive model comprises a random forest, neural network, naïve bayes, support vector machines, learning regression, k-nearest neighbors, k-means, decision tree, logistic regression, gradient boosting, or any combination thereof. Numbered embodiment 120 comprises the method as in any of embodiments 81-119, wherein enriching said one or more microbial extracellular vesicles improves an accuracy of said predictive model by at least 1%, at least 5%, at least 10%, at least 15%, or at least 20% when predicting said treatment of said subject. Numbered embodiment 121 comprises the method as in any of embodiments 81-120, wherein said subject comprises a non-human mammal or a human subject. Numbered embodiment 122 comprises the method as in any of embodiments 81-121, wherein said treatment comprises a repurposed treatment, which may or may not have been originally approved for targeting cancer. Numbered embodiment 123 comprises the method as in any of embodiments 81-122, wherein said treatment comprises a small molecule, a biologic, a probiotic, a virus, a bacteriophage, an immunotherapy, a broad-spectrum antibiotic, or any combination thereof. Numbered embodiment 124 comprises the method as in any of embodiments 81-123, wherein said probiotic comprises an engineered bacterium strain or ensemble of engineered bacteria. Numbered embodiment 125 comprises the method as in any of embodiments 81-122, wherein said treatment comprises an adjuvant given in combination with a primary treatment against said cancer to improve an efficacy of said primary treatment. Numbered embodiment 126 comprises the method as in any of embodiments 81-122, wherein said treatment comprises adoptive cell transfer to target microbial antigens associated with said cancer or cancer microenvironment. Numbered embodiment 127 comprises the method as in any of embodiments 81-122, wherein said treatment comprises a cancer vaccine that exploits microbial antigens associated with said cancer or cancer microenvironment. Numbered embodiment 128 comprises the method as in any of embodiments 81-122, wherein said treatment comprises a monoclonal antibody against microbial antigens associated with said cancer or cancer microenvironment. Numbered embodiment 129 comprises the method as in any of embodiments 81-122, wherein said treatment comprises an antibody-drug conjugate designed to at least partially target microbial antigens associated with said cancer or cancer microenvironment. Numbered embodiment 130 comprises the method as in any of embodiments 81-122, wherein said treatment comprises a multi-valent antibody, antibody fragment, or antibody derivative thereof designed to at least partially target one or more microbial antigens associated with said cancer or cancer microenvironment. Numbered embodiment 131 comprises the method as in any of embodiments 81-122, wherein said treatment comprises a targeted antibiotic against a particular kind of microbe or class of functionally or biologically similar microbes. Numbered embodiment 132 comprises the method as in any of embodiments 81-122, wherein two or more of the following treatment types are combined such that at least one type exploits said cancer microbial presence or abundance to enhance overall therapeutic efficacy: small molecules, biologics, engineered host-derived cell types, probiotics, engineered bacteria, natural-but-selective viruses, engineered viruses, and bacteriophages.

Numbered embodiment 133 comprises a method of identifying a treatment for a disease of a subject, comprising: providing a biological sample of a subject comprising one or more microbial extracellular vesicles and one or more non-microbial extracellular vesicles; enriching said one or more microbial extracellular vesicles with one or more first affinity reagents and said one or more non-microbial extracellular vesicles with one or more second affinity reagents, thereby generating one or more enriched microbial extracellular vesicles and one or more enriched non-microbial extracellular vesicles; detecting a first abundance of one or more molecular analytes from said enriched one or more microbial extracellular vesicles and a second abundance of one or more molecular analytes from said enriched one or more non-microbial extracellular vesicles; and identifying said treatment for said disease of said subject from an association between a combination of said first abundance of one or more molecular analytes and said second abundance of one or more molecular analytes, and a model of disease treatments associated with a third abundance of one or more molecular analytes from one or more microbial extracellular vesicles and a fourth abundance of one or more molecular analytes from one or more non-microbial extracellular vesicles. Numbered embodiment 134 comprises the method of embodiment 133, wherein detecting comprises quantifying said first abundance of one or more molecular analytes and said second abundance of one or more molecular analytes. Numbered embodiment 135 comprises the method as in embodiments 133 or 134, wherein said one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. Numbered embodiment 136 comprises the method as in any of embodiments 133-135, wherein said one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. Numbered embodiment 137 comprises the method as in any of embodiments 133-136, wherein said biological sample comprises a liquid biological sample, a tissue biological sample, or any combination thereof. Numbered embodiment 138 comprises the method as in any of embodiments 133-137, wherein said liquid biological sample comprises plasma, serum, whole blood, urine, cerebral spinal fluid, saliva, sweat, tears, lymphatic fluid, exhaled breath condensate, or any combination, any dilution, or processed fraction thereof. Numbered embodiment 139 comprises the method as in any of embodiments 133-138, wherein whole blood comprises white blood cells, plasma, red blood cells, platelets, or any combination thereof. Numbered embodiment 140 comprises the method as in any of embodiments 133-137, wherein the tissue biological sample comprises tissue homogenate. Numbered embodiment 141 comprises the method as in any of embodiments 133-140, wherein enriching comprises concentrating one or more microbial or one or more non-microbial cell wall molecular motifs. Numbered embodiment 142 comprises the method as in any of embodiments 133-141 wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof. Numbered embodiment 143 comprises the method as in any of embodiments 133-142, wherein said one or more first affinity reagents or said one or more second affinity reagents comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof. Numbered embodiment 144 comprises the method as in any of embodiments 133-143, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof. Numbered embodiment 145 comprises the method as in any of embodiments 133-144, wherein said derivatives thereof comprise an epitope tag, full-length recombinant innate immunity pattern recognition receptors, or recombinant soluble ectodomains thereof. Numbered embodiment 146 comprises the method as in any of embodiments 133-145, wherein said one or more antibodies, said aptamers, said single-chain variable fragments (scFv), and said single-chain antibodies comprise an epitope tag. Numbered embodiment 147 comprises the method as in any of embodiments 133-146, wherein said epitope tag comprises a N- or C-terminal 6×-histidine tag, green fluorescent protein (GFP), myc, hemagglutinin (HA), Fc fusion, biotin, or any combination thereof. Numbered embodiment 148 comprises the method as in any of embodiments 133-147, wherein said one or more first affinity reagents and said one or more second affinity reagents comprise a region to interact with said one or more microbial cell wall molecular motifs or said one or more non-microbial cell wall molecular motifs. Numbered embodiment 149 comprises the method as in any of embodiments 133-148, wherein enriching comprises incubating said liquid biological sample with a support comprising covalently immobilized affinity agents. Numbered embodiment 150 comprises the method as in any of embodiments 133-149, wherein said covalently immobilized affinity agents comprise a region that interacts with said one or more microbial cell wall molecular motifs or said one or more non-microbial cell wall molecular motifs. Numbered embodiment 151 comprises the method as in any of embodiments 133-150, wherein said supports comprise a magnetic bead, an agarose bead, non-magnetic latex, functionalized Sepharose, pH-sensitive polymers or any combinations thereof. Numbered embodiment 152 comprises the method as in any of embodiments 133-151, wherein said one or more second affinity reagents comprise polyclonal, monoclonal, recombinant antibodies, aptamers, single-chain variable fragment (scFv), single-chain antibodies, or any combination thereof. Numbered embodiment 153 comprises the method as in any of embodiments 133-152, wherein said one or more first affinity reagents or said one or more second affinity reagents are specific to mammalian antigens CD9, CD63, CD81, glypican 1 (GPC1), Mart-1, TYRP2, Human epidermal growth factor receptors (HER) family members, EpCAM, or any combination thereof. Numbered embodiment 154 comprises the method as in any of embodiments 133-153, wherein detecting comprises performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with said one or more molecular analytes of said one or more microbial extracellular vesicles or said one or more molecular analytes from said one or more non-microbial extracellular vesicles. Numbered embodiment 155 comprises the method as in any of embodiments 133-154, wherein nucleic acid sequencing comprises whole genome sequencing, shotgun sequencing, next generations sequencing, targeted sequencing, RNA sequencing, methylation sequencing, or any combination thereof. Numbered embodiment 156 comprises the method as in any of embodiments 133-155, wherein said model comprises a predictive model, wherein said predictive model is trained with one or more subjects' said third abundance of one or more molecular analytes from said one or more microbial extracellular vesicles, said fourth abundance of one or more molecular analytes from said one or more non-microbial extracellular vesicles, and a corresponding treatment for said disease of said one or more subjects. Numbered embodiment 157 comprises the method as in any of embodiments 133-156, wherein said predictive model is configured to receive said first abundance of one or more molecular analytes and said second abundance of one or more molecular analytes as an input and output a prediction of said treatment for said disease of said subject. Numbered embodiment 158 comprises the method as in any of embodiments 133-157, wherein said disease comprises cancer. Numbered embodiment 159 comprises the method as in any of embodiments 133-158, wherein said cancer comprises a stage I or stage II cancer. Numbered embodiment 160 comprises the method as in any of embodiments 133-159, wherein said cancer comprises bone, breast, lung, colon, brain, skin, ovary, pancreas, or any combination thereof type of cancer. Numbered embodiment 161 comprises the method as in any of embodiments 133-160, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 162 comprises the method as in any of embodiments 133-160, wherein said cancer comprises one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 163 comprises the method as in any of embodiments 133-162, wherein said predictive model comprises a machine learning model. Numbered embodiment 164 comprises the method as in any of embodiments 133-163, wherein said machine learning model comprises one or more machine learning models, a regularized machine learning model, an ensemble of machine learning models, or any combination thereof. Numbered embodiment 165 comprises the method as in any of embodiments 133-164, wherein said predictive model comprises a random forest, neural network, naïve bayes, support vector machines, linear regression, k-nearest neighbors, k-means, decision tree, logistic regression, gradient boosting, or any combination thereof predictive model. Numbered embodiment 166 comprises the method as in any of embodiments 133-165, wherein said subject comprises a human or a non-human mammal. Numbered embodiment 167 comprises the method as in any of embodiments 133-166, wherein enriching said one or more microbial extracellular vesicles and said one or more non-microbial extracellular vesicles improves an accuracy of said predictive model by at least 1%, at least 5%, at least 10%, at least 15%, or at least 20% when predicting said treatment for said cancer of said subject. Numbered embodiment 168 comprises the method as in any of embodiments 133-167, wherein an area under a receiver operating characteristic curve of said predictive model predicting said treatment for said disease of said subject is increase by at least 1%, at least 2%, at least 4%, at least 5%, or at least 10%, when said combination of said first abundance of one or more molecular analytes and said second abundance of one or more molecular analytes are provided as said input to said predictive model. Numbered embodiment 169 comprises the method as in any of embodiments 133-168, wherein said treatment comprises a repurposed treatment, which may or may not have been originally approved for targeting cancer. Numbered embodiment 170 comprises the method as in any of embodiments 133-169, wherein said treatment comprises a small molecule, a biologic, a probiotic, a virus, a bacteriophage, an immunotherapy, a broad-spectrum antibiotic, or any combination thereof. Numbered embodiment 171 comprises the method as in any of embodiments 133-170, wherein said probiotic comprises an engineered bacterium strain or ensemble of engineered bacteria. Numbered embodiment 172 comprises the method as in any of embodiments 133-169, wherein said treatment comprises an adjuvant given in combination with a primary treatment against said cancer to improve an efficacy of said primary treatment. Numbered embodiment 173 comprises the method as in any of embodiments 133-169, wherein said treatment comprises adoptive cell transfer to target microbial antigens associated with said cancer or cancer microenvironment. Numbered embodiment 174 comprises the method as in any of embodiments 133-169, wherein said treatment comprises a cancer vaccine that exploits microbial antigens associated with said cancer or cancer microenvironment. Numbered embodiment 175 comprises the method as in any of embodiments 133-169, wherein said treatment comprises a monoclonal antibody against microbial antigens associated with said cancer or cancer microenvironment. Numbered embodiment 176 comprises the method as in any of embodiments 133-169, wherein said treatment comprises an antibody-drug conjugate designed to at least partially target microbial antigens associated with said cancer or cancer microenvironment. Numbered embodiment 177 comprises the method as in any of embodiments 133-169, wherein said treatment comprises a multi-valent antibody, antibody fragment, or antibody derivative thereof designed to at least partially target one or more microbial antigens associated with said cancer or cancer microenvironment. Numbered embodiment 178 comprises the method as in any of embodiments 133-169, wherein said treatment comprises a targeted antibiotic against a particular kind of microbe or class of functionally or biologically similar microbes. Numbered embodiment 179 comprises the method as in any of embodiments 133-169, wherein two or more of the following treatment types are combined such that at least one type exploits said cancer microbial presence or abundance to enhance overall therapeutic efficacy: small molecules, biologics, engineered host-derived cell types, probiotics, engineered bacteria, natural-but-selective viruses, engineered viruses, and bacteriophages.

Numbered embodiment 180 comprises a method of training a predictive model, comprising: providing a biological sample of one or more subjects comprising one or more microbial extracellular vesicles, and an associated health classification of said one or more subjects; enriching said one or more microbial extracellular vesicles with one or more affinity reagents, thereby generating one or more enriched microbial extracellular vesicles; detecting one or more molecular analytes from said enriched one or more microbial extracellular vesicles; and training said predictive model with said one or more molecular analytes and said associated health classification of said one or more subjects. Numbered embodiment 181 comprises the method of embodiment 180, wherein said biological sample comprises non-microbial extracellular vesicles. Numbered embodiment 182 comprises the method as in embodiments 180 or 181, further comprising quantifying an abundance of said one or more molecular analytes. Numbered embodiment 183 comprises the method as in any of embodiments 180-182, wherein said one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof. Numbered embodiment 184 comprises the method as in any of embodiments 180-183, wherein said one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof. Numbered embodiment 185 comprises the method as in any of embodiments 180-184, wherein said biological sample comprises a liquid biological sample, tissue biological sample, or any combination thereof. Numbered embodiment 186 comprises the method as in any of embodiments 180-185, wherein said liquid biological sample comprises plasma, serum, whole blood, urine, cerebral spinal fluid, saliva, sweat, tears, lymphatic fluid, exhaled breath condensate or any dilution, or processed fraction thereof. Numbered embodiment 187 comprises the method as in any of embodiments 180-186, wherein said whole blood comprises plasma, white blood cells, red blood cells, platelets, or any combination thereof. Numbered embodiment 188 comprises the method as in any of embodiments 180-185, wherein said tissue biological sample comprises tissue homogenate. Numbered embodiment 189 comprises the method as in any of embodiments 180-188, wherein said one or more affinity reagents are configured to couple to one or more microbial cell wall molecular motifs or one or more non-microbial cell wall molecular motifs. Numbered embodiment 190 comprises the method as in any of embodiments 180-189, wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof. Numbered embodiment 191 comprises the method as in any of embodiments 180-190, wherein said one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof. Numbered embodiment 192 comprises the method as in any of embodiments 180-191, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof. Numbered embodiment 193 comprises the method as in any of embodiments 180-192, wherein said derivatives thereof comprise an epitope tag and full-length recombinant innate immunity pattern recognition receptors or recombinant soluble ectodomains thereof. Numbered embodiment 194 comprises the method as in any of embodiments 180-193, wherein said epitope tag comprises a N- or C-terminal 6×-histidine tag, green fluorescent protein (GFP), myc, hemagglutinin (HA), Fc fusion, biotin, or any combination thereof. Numbered embodiment 195 comprises the method as in any of embodiments 180-191, wherein said one or more antibodies, aptamers, single-chain variable fragments (scFv), or said single-chain antibodies comprise an epitope tag. Numbered embodiment 196 comprises the method as in embodiments 180-191, or 195, wherein said epitope tag comprises a N- or C-terminal 6×-histidine tag, green fluorescent protein (GFP), myc, hemagglutinin (HA), Fc fusion, biotin or any combination thereof. Numbered embodiment 197 comprises the method as in any of embodiments 180-196, wherein said one or more affinity reagents comprise a region to interact with said one or more microbial cell wall molecular motifs or said one or more non-microbial cell wall molecular motifs. Numbered embodiment 198 comprises the method as in any of embodiments 180-197, wherein said enriching comprises contacting said liquid biological sample with a support comprising covalently immobilized affinity agents. Numbered embodiment 199 comprises the method as in any of embodiments 180-198, wherein said covalently immobilized affinity agents comprise a region that interacts with said one or more microbial cell wall molecular motifs or said one or more non-microbial cell wall molecular motifs. Numbered embodiment 200 comprises the method as in any of embodiments 180-199, wherein said supports comprise a magnetic bead, an agarose bead, non-magnetic latex, functionalized Sepharose, pH-sensitive polymers or any combinations thereof. Numbered embodiment 201 comprises a method as in any of embodiment 180-200, wherein said enriching comprises: contacting said liquid biological sample with said one or more affinity reagents to form capture reagent-molecular motif interaction complexes; contacting said capture reagent-molecular motif interaction complexes with a support; and separating said support from said liquid biological sample to concentrate said capture reagent-molecular motif interaction complexes. Numbered embodiment 202 comprises the method as in any of embodiments 180-201, wherein said one or more affinity reagents comprise an epitope tag. Numbered embodiment 203 comprises the method as in any of embodiments 180-202, wherein said support comprises an epitope tag recognition surface. Numbered embodiment 204 comprises the method as in any of embodiments 180-203, wherein said epitope tag recognition surface comprises streptavidin, antibodies specific for 6×-histidine tag, green fluorescent protein (GFP), myc, hemagglutinin (HA), biotin, or any combination thereof. Numbered embodiment 205 comprises the method as in any of embodiments 180-204, wherein said epitope tag recognition surface comprises an anti-species antibody. Numbered embodiment 206 comprises the method as in any of embodiments 180-205, wherein said detecting comprises nucleic acid sequencing, polymerase chain reaction (PCR)-based, or hybridization-based analysis for nucleic acid analytes. Numbered embodiment 207 comprises the method as in any of embodiments 180-206, wherein said nucleic acid sequencing comprises whole genome sequencing, shotgun sequencing, next generations sequencing, targeted sequencing, RNA sequencing, methylation sequencing, or any combination thereof. Numbered embodiment 208 comprises the method as in any of embodiments 180-207, wherein said detecting comprises performing mass spectrometry analyses, liquid chromatography-mass spectrometry (LC-MS), or high-performance liquid chromatography (HPLC). Numbered embodiment 209 comprises the method as in any of embodiments 180-208, wherein said detecting comprises performing immunoassay analysis. Numbered embodiment 210 comprises the method as in any of embodiments 180-209, wherein said trained predictive model is configured to receive one or more molecular analytes of a subject as an input and output a predicted disease of said subject, a treatment for said disease of said subject, or any combination thereof. Numbered embodiment 211 comprises the method as in any of embodiments 180-210, wherein said disease comprises cancer. Numbered embodiment 212 comprises the method as in any of embodiments 180-211, wherein said cancer comprises a stage I or stage II cancer. Numbered embodiment 213 comprises the method as in any of embodiments 180-212, wherein said cancer comprises bone, breast, lung, colon, brain, skin, ovary, pancreas, or any combination thereof type of cancer. Numbered embodiment 214 comprises the method as in any of embodiments 180-213, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 215 comprises the method as in any of embodiments 180-214, wherein said cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 216 comprises the method as in any of embodiments 180-215, wherein said trained predictive model is configured to predict one or more cancers, one or more subtypes of cancer, the anatomical locations of one or more cancers, or any combination thereof of said subject. Numbered embodiment 217 comprises the method as in any of embodiments 180-216, wherein said trained predictive model is configured to predict a stage of said cancer, prognosis of said cancer, mutation status of said cancer, future immunotherapy response of said cancer, an optimal therapy to treat said cancer, or any combination thereof. Numbered embodiment 218 comprises the method as in any of embodiments 180-217, wherein said trained predictive model is configured to predict said cancer among one or more cancer types of said subject to identify a specific cancer type of said one or more cancer types. Numbered embodiment 219 comprises the method as in any of embodiments 180-218, wherein said trained predictive model comprises a machine learning model, wherein said machine learning model comprises a regularized machine learning model, ensemble of machine learning models, or any combination thereof. Numbered embodiment 220 comprises the method as in any of embodiments 180-219, wherein said trained predictive model comprises a random forest, neural network, naïve bayes, support vector machines, learning regression, k-nearest neighbors, k-means, decision tree, logistic regression, gradient boosting, or any combination thereof. Numbered embodiment 221 comprises the method as in any of embodiments 180-220, wherein enriching said microbial extracellular vesicles improves an accuracy of said trained predictive model by at least 1%, at least 5%, at least 10%, at least 15%, or at least 20% when predicting said cancer of said subject. Numbered embodiment 222 comprises the method as in any of embodiments 180-221, wherein said subject comprises a non-human mammal or a human subject. Numbered embodiment 223 comprises the method as in any of embodiments 180-222, wherein said health classification comprises cancerous, non-cancerous disease, or non-cancerous non-disease.

### EXAMPLES

### Example 1: Differential Centrifugation of Plasma and Orthogonal Characterization of Cell-Free Nucleic Acids and Vesicular Content from Each Fraction

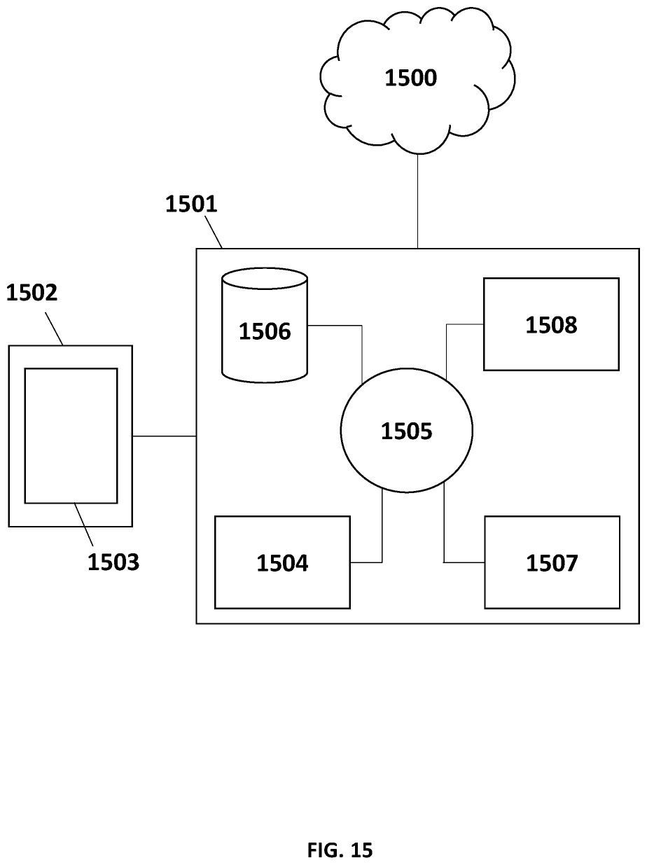

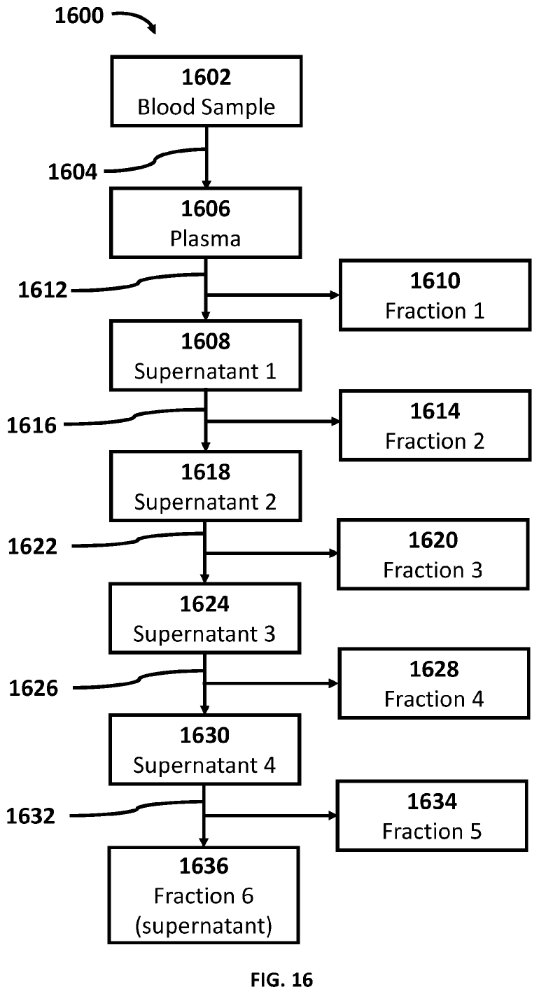

The presence of microbial extracellular vesicles and the number of unique microbial genera in blood plasma of small cell lung cancer (SCLC) patients was analyzed by differential plasma centrifugation 1600, as shown in FIG. 16. Blood samples of SCLC patients 1602 were subjected to centrifugation at 3,000 rotations per minute (rpm) for 10 minutes 1604 yielding a volume of about 1 mL of plasma 1606. The plasma was then subjected to increasing centrifugation speed and/or specific treatment of the supernatant fraction (1610-1636) to generate sequential precipitation of different plasma components (1610, 1614, 1620, 1628, 1634). The serial plasma centrifugation steps are briefly described. After isolating plasma 1606 from the blood sample(s) 1602, the plasma was then centrifuged at 500 g for 5 minutes (1612) to precipitate a first fraction 1610 of cells and cellular debris at approximately 7-10 kilobase (kb) length of nucleic acid molecules (e.g., DNA). The resulting supernatant 1608, was then centrifuged at 2,000 g for 10 minutes (1616) to precipitate a second fraction (1614) of small cell debris and apoptotic bodies of sizes from about 1-5 micrometers (μm). The resulting second supernatant (1618) was then centrifuged at 10,000 g for 30 minutes (1622) to precipitate large microvesicles of sizes of about 100 nanometer (nm)-1 μm (1620). The resulting third supernatant (1624) was then treated with thrombin (1626) to precipitate fibrin (1628). The resulting fourth supernatant (1630) was then incubated overnight at 4 degrees Celsius with ExoQuick reagent, then centrifuged at 1,500 g for 30 minutes (1632) to separate human exosomes of sizes of about 20 to 200 nm (1634) from a remaining supernatant expected to contain truly cell-free DNA of average length 140-160 base pairs (1636).

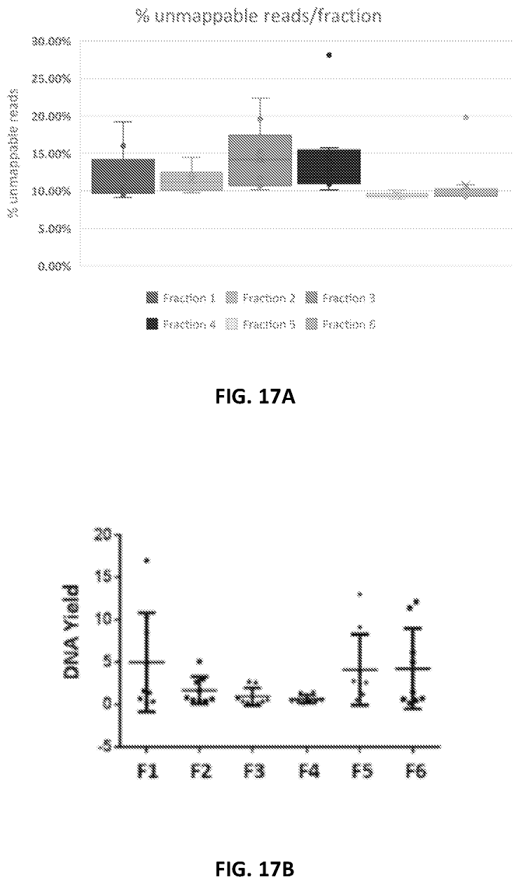

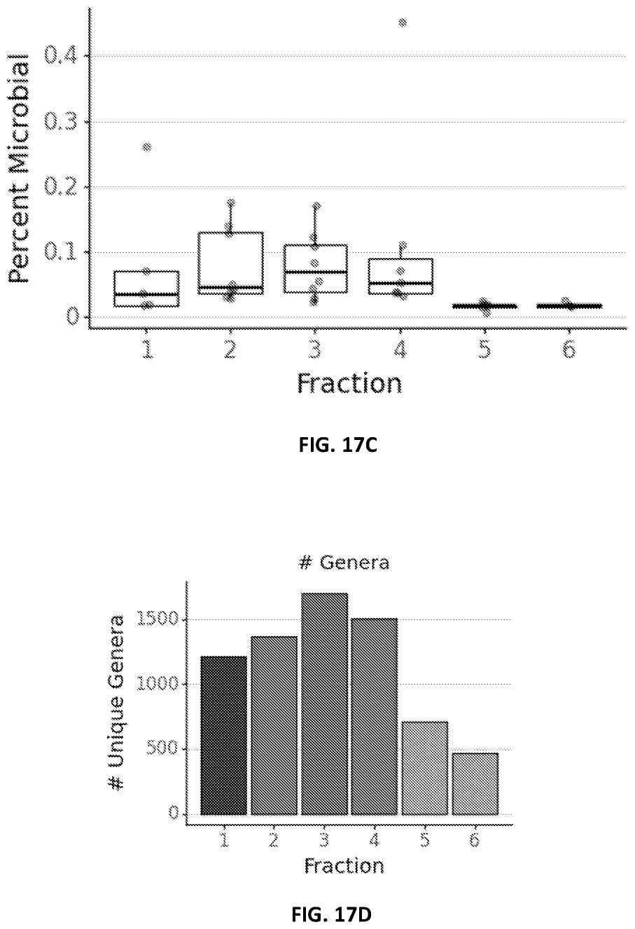

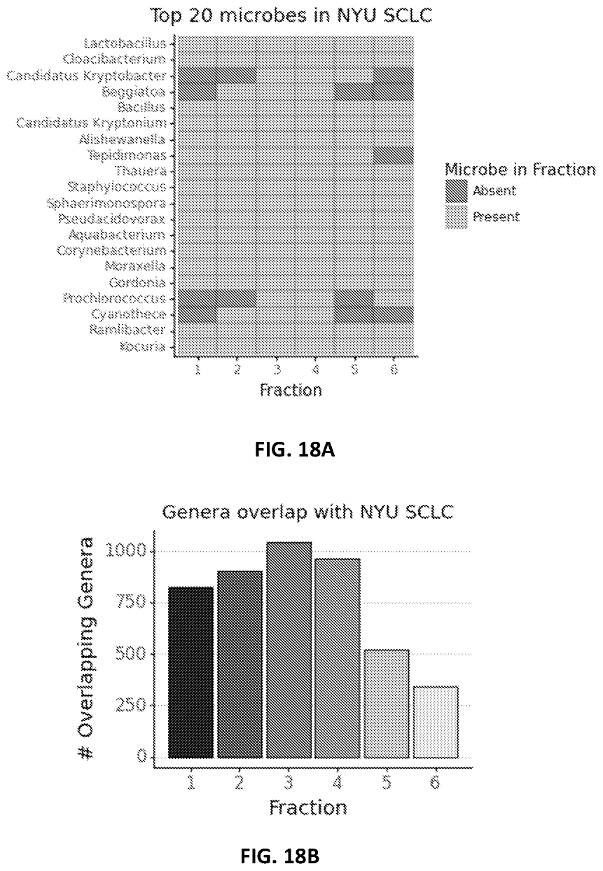

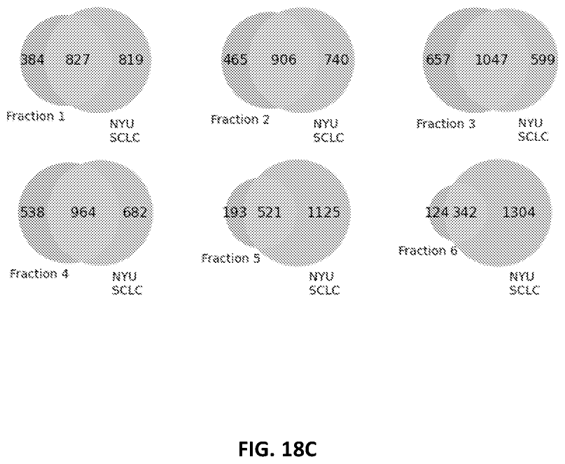

The plasma components isolated as described above were then analyzed for the presence and abundance of microbial nucleic acids, with the results shown in FIGS. 17A-17C, and compared to a second in-house SCLC dataset from New York University shown in FIGS. 18A-18B. The various plasma components isolated in the various fractions, as described above, were prepared in nucleic acid sequencing libraries for next generation sequencing (e.g., sequencing-by-synthesis). The DNA yield (ng/mL) per plasma fraction was measured and is shown in FIG. 17B. After sequencing the various nucleic acid molecules of each fraction, the sequencing reads were mapped to a human reference genome (hg19). The percentage of unmapped nucleic acid sequencing reads for each fraction, indicating non-human i.e., microbial sequencing reads, are shown in FIG. 17A. From the graph shown in FIG. 17A, it appears that although fractions 3 and 4 contained the lowest DNA, fractions 3 and 4 had the highest percentage of unmappable (i.e., non-human) reads, suggesting that these fractions are enriched in microbial DNA.

To further validate the presence of enriched microbial DNA in fractions 3 and 4, the raw nucleic acid sequencing reads from each fraction were further analyzed with respect to microbial composition of the various fractions and whether the microbial genre identified are stable features of a SCLC plasma microbiome across related SCLC datasets. Microbial composition was determined by aligning the raw nucleic acid sequencing reads from each fraction to a human reference genome database (hg38) to obtain human-filter reads. Reads that did not map to the human genome were then aligned to the Web of Life microbial reference genome database to determine the percent of microbial reads present in each fraction, the results of which are shown in FIG. 17C. From FIG. 17, it can be seen that fractions 3 and 4 contain the highest percent of microbial reads compared to all other fractions. Furthermore, the number of unique genera of microbes present in each fraction was calculated from the aligned sequencing reads aligned to the Web of Life microbial reference genome database, as shown in FIG. 17D. From FIG. 17D, it can be seen that fractions 3 and 4 have the highest number of unique microbial genera compared to the other fractions.

To determine the whether the identified microbial signature in fractions 3 and 4 are stable features of a SCLC plasma microbiome, the microbial signatures across all fractions were compared to an independent database of microbial plasma signatures from SCLC patients collected at New York University (hereinafter “NYU SCLC dataset”) (FIGS. 18A-18D). FIG. 18A shows a presence/absence heatmap of identified genera across the plasma fractions, described above and the microbes identified in the NYU SCLC dataset. From FIG. 18A it can be seen that fractions 3 and 4 were found to have all the top 20 microbes in the NYU SCLC dataset compared to fractions 1, 2, 5, and 6. Moreover, the overlap between the two dataset's microbial genera was also compared, as shown in FIGS. 18B-18C. From FIGS. 18B-18C it can be seen that there is significant genera overlap between the two datasets within fraction 3 and 4, represented in both a number of overlapping genera per fraction (FIG. 18B) and in a Venn diagram representation as can be seen in FIG. 18C, indicating that the microbial signature of fraction 3 and 4 are stable features of a SCLC plasma microbiome.

### Example 2: Flow Cytometry-Based Analysis of Human Protein Markers Exposed on Vesicular Membranes

Based on the analysis of microbial abundance in each fraction of Example 1, fractions 3 and 4 are enriched in non-human DNA. Therefore, fractions 3, 4, and 5 (human exosomes) can server as the basis for flow-cytometry-based diagnostic, where cancer-derived human exosomes and microbial extracellular vesicles are quantified on basis of disease-characteristic protein and/or glycan biomarkers.

For the evaluation of human protein markers exposed on vesicular membranes, flow cytometry analysis is performed on the pellet fractions from fractions 3 and 5, which are resuspended in 100 μL of phosphate buffered saline and incubated with anti-CD63, CD9, CD81, and hSP70 specific monoclonal antibodies with fluorescent direct labeling.

For the evaluation of microbial vesicle surface markers, surface staining can be directed to canonical microbial molecular features (e.g., LPS, LTA, n-acetylglucosamine) or can be directed to novel glycan features identified by the methods of the present invention (Example 3 and 4).

In addition, the nucleic acid content (DNA and/or RNA) of the isolated MEVs is purified using the QIAmp Circulating Nucleic Acid Kits. Final DNA eluents (50 μL) and is quantified using Qubit HS dsDNA assay and their DNA fragment size distribution is analyzed on a 4200 TapeStation instruction. The cfDNA is then used for the generation of NGS metagenomic libraries for shotgun sequencing analysis on a NovaSeq6000 instrument. The sequencing data analysis is successively performed to estimate the relative sample composition of human and microbial DNA and determine taxonomy of the most abundance microbial genome equivalents. At this stage, the percentage of circulating tumor DNA (relative to the total cfDNA) in each plasma sample and plasma fraction recovered is also estimated. The sequencing data generated by this method is then further analyzed by the methods of the invention, described elsewhere herein.

### Example 3: Cocktail of Anti-LTA and Anti-LPS Antibodies for the Enrichment of Microbial Extracellular Vesicles

The separation of microbial extracellular vesicles from plasma is achieved by targeting the major bacterial cell-wall constituents via immunoaffinity methods, as described elsewhere herein. Primary monoclonal antibodies that have been engineered to recognize the core region of lipopolysaccharides (LPS) and lipoteichoic acid (LTA) are employed in this example. A polyclonal antibody targeting the Lipid A domain of the LPS, and an anti-LTA monoclonal antibody are tested singularly, or as a cocktail mixture for the immunoprecipitation of microbial vesicles from plasma. The same antibodies are also tested in the presence of LPS and LTA positive controls (alone in solution, or spiked-in plasma), respectively.

The immunoprecipitation is performed with antibody-agarose conjugate prepared from protein A or G agarose/Sepharose beads. 100 mg of beads are incubated in 1 mL 0.1 M PBS for 1 hour, then centrifuged, and the supernatant is discarded. The precipitate is resuspended in 1 mL PBS 0.1% BSA and mixed for one hour on rotation, finally rinsed twice in PBS. After removing this supernatant, a volume of 400 μL of buffer containing protease inhibitors is added. This slurry is stored at 4° C. until ready to proceed. A volume of 100 μL of slurry of Protein A-, or G-conjugate is mixed with 10 μL of primary antibody, incubated for 4 hours at 4° C. on shaking, successively centrifuged at 1,500 g for 2 min at 4° C. and the supernatant is discarded. Due to the high immunoglobulin concentration of plasma, the anti-LPS and/or anti-LTA antibodies are first covalently immobilized to Protein A, Protein G or Protein A/G beads via chemical crosslinking with dimethyl pimelidate dihydrochloride or other short-length homobifunctional, amine-reactive crosslinkers. The crosslinked antibody-Protein A/G beads are pelleted and washed twice with 1 mL of an appropriate buffer and centrifuged at 3,000 g for 2 min at 4° C. The bead/antibody conjugate mixture is then added to 1 mL of plasma sample and incubated at 4° C. under rotary agitation overnight to allow formation of the antibody/protein target complex. At the end of the incubation, the tubes are centrifuged, the supernatant discarded, and the beads washed with buffer three times to remove non-specific binding and centrifuged at 4° C. in between each wash to collect and discard the supernatant. The elution of the antibody/protein marker off the beads is performed using 50 μL of a low-pH glycine buffer: 0.2 M glycine pH 2.6 (1:1) by incubating the sample for 10 minutes with frequent agitation before gentle centrifugation. The eluted sample is immediately neutralized with Tris, pH 8.0-8.5.

The eluted complex contains intact extracellular bodies (larger and smaller vesicles) associated with the targeted LPS and LTA analytes. The sample is now ready for downstream analyses including nanoparticles tracking analysis, electron microscopy, flow cytometry, and DNA isolation for the preparation of metagenomic libraries and shallow shotgun sequencing analysis, as described in Examples 1 and 2.

### Example 4: Lectin Microarray-Mediated Identification of MEV Glycan Signatures

A lectin-based glycan profiling in plasma samples is achieved with the use of a lectin microarray assay (e.g., Creative Proteomics, NY, USA or Raybiotech Glycan Array 300), that utilizes a panel of lectins immobilized on well-defined solid surface for high-throughput analysis of glycans and glycoproteins, where the target molecules in the sample are fluorescently labeled.

MEVs isolated by the method outlined in Example 1 and 2 are washed in 1×PBS and incubated with NHS-activated fluorescent dye (e.g., NHS-fluorescein) to fluorescently label the MEVs. Labeled MEVs are then incubated with lectin arrays to determine what carbohydrate structures are present on MEVs. The identified lectins from the spots of the greatest fluorescence intensity can then be used as specific MEV enrichment reagents. For example, immobilization of the MEV-specific lectins to a solid surface can enable direct lectin-mediated enrichment of MEVs from a complex plasma sample on the basis of lectin-MEV glycan recognition events.

### Example 5: Lectin Microarray-Mediated Identification of MEV Glycan Signatures, Cancer vs. Non-Caner Conditions

Using the above MEV labeling method from Example 4, MEVs isolated from subjects with cancer vs. subjects with non-cancer conditions of the same organ/tissue (e.g., small cell lung cancer vs. pulmonary granulomas) are labeled with fluorophores as above and incubated with lectin microarrays. The specific glycans identified by the lectin array (cancer vs. non-cancer) can be further exploited as quantifiable biomarkers to discriminate cancer vs. non-cancer conditions or can be utilized as a means of targeted enrichment of cancer vs. non-cancer MEVs, as shown in Example 4. Once isolated, the glycan targeted MEVs can be further analyzed via, for example, NGS analysis.

### Example 6: Engineered Toll-Like Receptor (TLR) Proteins for the Enrichment of Microbial Vesicles Via Immunoaffinity Capture

Isolation and purification of microbial vesicles is achieved via in-solution immunoaffinity capture using recombinant TLR proteins, mammalian innate immunity receptors that engage structural motifs of the microbial lipopolysaccharide (LPS, gram negative bacteria) and lipoteichoic acid (LTA, gram positive bacteria). The portion of a gene expressing the N-terminal region of a human TLR extracellular domain is fused to the Fc portion of a human immunoglobulin (domain 3 and 4 of the Ig heavy-chain) as an insert within an optimized plasmid construct. The plasmid construct is used to transfect human cells, e.g., HEK293, which can express the TLR-Fc chimeric protein. Purified TLR-Fc proteins are employed for the immunoaffinity capture of entire MEV as the binding between TLR proteins and their specific ligands occurs. Ideally, a cocktail of different TLR sub-families are used to increase the diversity of the targeted MEV. For example, a mixture of TLR-2, TLR-4, TLR-5, or TLR-1/TLR-2 and TLR-2/TLR-6 heterodimers could be used. Due to the high immunoglobulin concentration of plasma, TLR-Fc complexes are first covalently immobilized to Protein A, Protein G or Protein A/G beads via chemical crosslinking with dimethyl pimelidate dihydrochloride or other short-length homobifunctional, amine-reactive crosslinkers. The resulting TLR-Fc-Protein A/G bead complexes are then incubated with plasma to form TLR-MEV complexes. The resulting TLR-MEV complexes are then separated from the solution via magnetic concentration or centrifugation to pellet the functionalized beads. After capture, MEV can be isolated and the dissociation of TLR-MEV complexes can be performed by a cleavage protease enzyme that acts with high specificity on a cleavage site inserted between the Fc-tag and the TLR protein in the initial plasmid construct design (examples are: enterokinase (EK), HRV-3C (human rhinovirus protease), TEV). Alternatively, the isolated TLR-MEV complexes can be used directly for nucleic acid (or other analyte) isolation.

What is claimed is:

1. A method of identifying a disease of a subject, comprising:
(a) providing a biological sample from a subject comprising one or more microbial extracellular vesicles;
(b) enriching said one or more microbial extracellular vesicles with one or more affinity reagents;
(c) detecting one or more molecular analytes from said one or more microbial extracellular vesicles; and
(d) identifying said disease of said subject based on said one or more molecular analytes from said one or more microbial extracellular vesicles.

2. The method of claim 1, wherein said biological sample comprises non-microbial extracellular vesicles.

3. The method of claim 2, further comprising quantifying an abundance of said one or more molecular analytes.

4. The method of claim 1, wherein said one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof.

5. The method of claim 2, wherein said one or more molecular analytes of said one or more non-microbial extracellular comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof.

6. The method of claim 1, wherein said biological sample comprises a liquid biological sample, tissue biological sample, or any combination thereof.

7. The method of claim 6, wherein said liquid biological sample comprises plasma, serum, whole blood, urine, cerebral spinal fluid, saliva, sweat, tears, lymphatic fluid, exhaled breath condensate or any dilution, or processed fraction thereof.

8. The method of claim 7, wherein said whole blood comprises plasma, white blood cells, red blood cells, platelets, or any combination thereof.

9. The method of claim 6, wherein said tissue biological sample comprises tissue homogenate.

10. The method of claim 1, wherein said one or more affinity reagents are configured to couple to one or more microbial or one or more non-microbial cell wall molecular motifs.

11. The method of claim 10, wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof.

12. The method of claim 1, wherein said one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof.

13. The method of claim 12, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof.

14. The method of claim 13, wherein said derivatives thereof comprise an epitope tag and full-length recombinant innate immunity pattern recognition receptors or recombinant soluble ectodomains thereof.

15. The method of claim 14, wherein said epitope tag comprises a N- or C-terminal 6×-histidine tag, green fluorescent protein (GFP), myc, hemagglutinin (HA), Fc fusion, biotin or any combination thereof.

16. The method of claim 12, wherein said one or more antibodies, aptamers, single-chain variable fragments (scFv), or said single-chain antibodies comprise an epitope tag.

17. The method of claim 16, wherein said epitope tag comprises a N- or C-terminal 6×-histidine tag, green fluorescent protein (GFP), myc, hemagglutinin (HA), Fc fusion, biotin or any combination thereof.

18. The method of claim 1, wherein said one or more affinity reagents comprise a region to interact with said one or more microbial cell wall molecular motifs or said one or more non-microbial cell wall molecular motifs.

19. The method of claim 1, wherein said enriching comprises contacting said biological sample with a support comprising covalently immobilized affinity agents.

20. The method of claim 19, wherein said covalently immobilized affinity agents comprise a region that interacts with said one or more microbial cell wall molecular motifs or said one or more non-microbial cell wall molecular motifs.

21. The method of claim 19, wherein said supports comprise a magnetic bead, an agarose bead, non-magnetic latex, functionalized Sepharose, pH-sensitive polymers or any combination thereof.

22. The method of claim 1, wherein said enriching comprises:
(a) contacting said biological sample with said one or more affinity reagents to form capture reagent-molecular motif interaction complexes;
(b) contacting said capture reagent-molecular motif interaction complexes with a support; and
(c) separating said support from said biological sample to concentrate said capture reagent-molecular motif interaction complexes.

23. The method of claim 1, wherein said one or more affinity reagents comprise an epitope tag.

24. The method of claim 19, wherein said support comprises an epitope tag recognition surface.

25. The method of claim 24, wherein said epitope tag recognition surface comprises streptavidin, antibodies specific for 6×-histidine tag, green fluorescent protein (GFP), myc, hemagglutinin (HA), biotin, or any combination thereof.

26. The method of claim 25, wherein said epitope tag recognition surface comprises an anti-species antibody.

27. The method of claim 1, wherein said detecting comprises nucleic acid sequencing, polymerase chain reaction (PCR), or hybridization-based analysis of the one or more molecular analytes.

28. The method of claim 27, wherein said nucleic acid sequencing comprises whole genome sequencing, shotgun sequencing, next generations sequencing, targeted sequencing, RNA sequencing, methylation sequencing, or any combination thereof.

29. The method of claim 27, wherein said detecting comprises performing mass spectrometry analyses, liquid chromatography-mass spectrometry (LC-MS), or high-performance liquid chromatography (HPLC).

30. The method of claim 1, wherein said detecting comprises performing immunoassay analysis.

31. The method of claim 1, wherein said disease comprises cancer.

32. The method of claim 31, wherein said cancer comprises a stage I or stage II cancer.

33. The method of claim 31, wherein said cancer comprises bone, breast, lung, colon, brain, skin, ovary, pancreas, or any combination thereof type of cancer.

34. The method of claim 31, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

35. The method of claim 31, wherein said cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

36. The method of claim 1, wherein said identifying said cancer comprises predicting said cancer by a predictive model, wherein said predictive model is trained with one or more subjects' one or more molecular analytes obtained from one or more microbial extracellular vesicles and an associated disease of said one more subjects.

37. The method of claim 36, wherein said predictive model is configured to receive said one or more molecular analytes of said subject as an input, and output said disease of said subject.

38. The method of claim 36, wherein said predictive model is configured to predict one or more cancers, one or more subtypes of cancer, the anatomical locations of one or more cancers, or any combination thereof of said subject.

39. The method of claim 36, wherein said predictive model is configured to predict a stage of said cancer, prognosis of said cancer, mutation status of said cancer, future immunotherapy response of said cancer, an optimal therapy to treat said cancer, or any combination thereof.

40. The method of claim 36, wherein said predictive model is configured to predict said cancer among one or more cancer types of said subject to identify a specific cancer type of said one or more cancer types.

41. The method of claim 36, wherein said predictive model comprises a machine learning model, wherein said machine learning model comprises a regularized machine learning model, ensemble of machine learning models, or any combination thereof.

42. The method of claim 36, wherein said predictive model comprises a random forest, neural network, naïve bayes, support vector machines, learning regression, k-nearest neighbors, k-means, decision tree, logistic regression, gradient boosting, or any combination thereof.

43. The method of claim 36, wherein enriching said microbial extracellular vesicles improves an accuracy of said predictive model by at least 1%, at least 5%, at least 10%, at least 15%, or at least 20% when predicting said cancer of said subject.

44. The method of claim 1, wherein said subject comprises a non-human mammal or a human subject.

45. A method of identifying a disease of a subject comprising:
(a) providing biological sample of a subject comprising one or more microbial extracellular vesicles and one or more non-microbial extracellular vesicles;
(b) enriching said one or more microbial extracellular vesicles with one or more first affinity reagents and said one or more non-microbial extracellular vesicles with one or more second affinity reagents, thereby generating one or more enriched microbial extracellular vesicles and one or more enriched non-microbial extracellular vesicles;
(c) detecting a first abundance of one or more molecular analytes from said enriched one or more microbial extracellular vesicles and a second abundance of one or more molecular analytes from said enriched one or more non-microbial extracellular vesicles; and
(d) identifying said disease of said subject from an association between a combination of said first abundance of one or more molecular analytes and said second abundances of one or more molecular analytes, and a model of diseases associated with a third abundance of one or more molecular analytes from one or more microbial extracellular vesicles and a fourth abundance of one or more molecular analytes from one or more non-microbial extracellular vesicles.

46. The method of claim 45, wherein detecting comprises quantifying said first abundance of one or more molecular analytes and said second abundance of one or more molecular analytes.

47. The method of claim 45, wherein said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof.

48. The method of claim 45, wherein said one or more molecular analytes of said one or more non-microbial extracellular vesicles comprises cell-free non-microbial DNA, cell-free non-microbial RNA, non-microbial DNA, non-microbial RNA, non-microbial proteins, non-microbial metabolites, non-microbial lipids, non-microbial glycans, or any combination thereof.

49. The method of claim 45, wherein said biological sample comprises a liquid biological sample, tissue biological sample, or any combination thereof.

50. The method of claim 49, wherein said liquid biological sample comprises plasma, serum, whole blood, urine, cerebral spinal fluid, saliva, sweat, tears, lymphatic fluid, exhaled breath condensate, or any combination, any dilution, or processed fraction thereof.

51. The method of claim 50, wherein said whole blood comprises white blood cells, plasma, red blood cells, platelets, or any combination thereof.

52. The method of claim 49, wherein said tissue biological sample comprises tissue homogenate.

53. The method of claim 45, wherein enriching comprises concentrating one or more microbial cell wall molecular motifs or one or more non-microbial cell wall molecular motifs.

54. The method of claim 53, wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof.

55. The method of claim 45, wherein said one or more first affinity reagents or said one or more second affinity reagents comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof.

56. The method of claim 55, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof.

57. The method of claim 56, wherein said derivatives thereof comprise an epitope tag, full-length recombinant innate immunity pattern recognition receptors, or recombinant soluble ectodomains thereof.

58. The method of claim 55, wherein said one or more antibodies, said aptamers, said single-chain variable fragments (scFv), and said single-chain antibodies comprise an epitope tag.

59. The method of claim 58, wherein said epitope tag comprises a N- or C-terminal 6×-histidine tag, green fluorescent protein (GFP), myc, hemagglutinin (HA), Fc fusion, biotin or any combination thereof.

60. The method of claim 45, wherein said one or more first affinity reagents and said one or more second affinity reagents comprise a region to interact with said one or more microbial cell wall molecular motifs or said one or more non-microbial cell wall molecular motifs.

61. The method as in any of claim 45, wherein enriching comprises incubating said biological sample with a support comprising covalently immobilized affinity agents.

62. The method of claim 61, wherein said covalently immobilized affinity agents comprise a region that interacts with said one or more microbial cell wall molecular motifs or said one or more non-microbial cell wall molecular motifs.

63. The method of claim 61, wherein said supports comprise a magnetic bead, an agarose bead, non-magnetic latex, functionalized Sepharose, pH-sensitive polymers or any combinations thereof.

64. The method of claim 45, wherein said one or more first affinity reagents or said one or more second affinity reagents are specific to mammalian antigens CD9, CD63, CD81, glypican 1 (GPC1), Mart-1, TYRP2, Human epidermal growth factor receptors (HER) family members, EpCAM, or any combination thereof.

65. The method of claim 45, wherein detecting comprises performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with said one or more molecular analytes of said one or more microbial extracellular vesicles or said one or more molecular analytes from said one or more non-microbial extracellular vesicles.

66. The method of claim 65, wherein nucleic acid sequencing comprises whole genome sequencing, shotgun sequencing, next generations sequencing, targeted sequencing, RNA sequencing, methylation sequencing, or any combination thereof.

67. The method of claim 45, wherein said model comprises a predictive model, wherein the predictive model is trained with one or more subjects' said third abundance of one or more molecular analytes from said one or more microbial extracellular vesicles, said fourth abundance of one or more molecular analytes from said one or more non-microbial extracellular vesicles, and a corresponding disease of said one or more subjects.

68. The method of claim 67, wherein said predictive model is configured to receive said first abundance of one or more molecular analytes and said second abundance of one or more molecular analytes as an input and output a prediction of said disease of said subject.

69. The method of claim 45, wherein said disease comprises cancer.

70. The method of claim 69, wherein said cancer comprises a stage I or stage II cancer.

71. The method of claim 69, wherein said cancer comprises bone, breast, lung, colon, brain, skin, ovary, pancreas, or any combination thereof type of cancer.

72. The method of claim 67, wherein said predictive model is configured to predict one or more cancers, one or more subtypes of cancer, anatomical locations of one or more cancers, or any combination thereof in said subject.

73. The method of claim 69, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

74. The method of claim 69, wherein said cancer comprises one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

75. The method of claim 67, wherein said predictive model comprises a machine learning model.

76. The method of claim 75, wherein said machine learning model comprises one or more machine learning models, a regularized machine learning model, an ensemble of machine learning models, or any combination thereof.

77. The method of claim 67, wherein said predictive model comprises a random forest, neural network, naïve bayes, support vector machines, linear regression, k-nearest neighbors, k-means, decision tree, logistic regression, gradient boosting, or any combination thereof predictive model.

78. The method of claim 45, wherein said subject comprises a human or a non-human mammal.

79. The method of claim 67, wherein enriching said one or more microbial extracellular vesicles and said one or more non-microbial extracellular vesicles improves an accuracy of said predictive model by at least 1%, at least 5%, at least 10%, at least 15%, or at least 20% when predicting said cancer of said subject.

80. The method of claim 67, wherein an area under a receiver operating characteristic curve of said predictive model when predicting said disease of said subject is increase by at least 1%, at least 2%, at least 4%, at least 5%, or at least 10%, when said combination of said first abundance of one or more molecular analytes and said second abundance of one or more molecular analytes are provided as said input to said predictive model.

81. A method of identifying a treatment for a disease of a subject, comprising:
(a) providing a biological sample of a subject comprising one or more microbial extracellular vesicles;
(b) enriching said one or more microbial extracellular vesicles with one or more affinity reagents, thereby generating one or more enriched microbial extracellular vesicles;
(c) detecting one or more molecular analytes from said one or more enriched microbial extracellular vesicles; and
(d) identifying said treatment for said disease of said subject based on said one or more molecular analytes.

82. The method of claim 81, wherein said biological sample comprises non-microbial extracellular vesicles and said one or more microbial extracellular vesicles.

83. The method of claim 81, further comprising quantifying an abundance of said one or more molecular analytes.

84. The method of claim 81, wherein said one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof.

85. The method of claim 82, wherein said one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof.

86. The method of claim 81, wherein said biological sample comprises a liquid biological sample, tissue biological sample, or any combination thereof.

87. The method of claim 86, wherein said liquid biological sample comprises plasma, serum, whole blood, urine, cerebral spinal fluid, saliva, sweat, tears, lymphatic fluid, exhaled breath condensate or any dilution, or processed fraction thereof.

88. The method of claim 87, wherein said whole blood comprises plasma, white blood cells, red blood cells, platelets, or any combination thereof.

89. The method of claim 86, wherein said tissue biological sample comprises tissue homogenate.

90. The method of claim 81, wherein said one or more affinity reagents are configured to couple to one or more microbial or one or more non-microbial cell wall molecular motifs.

91. The method of claim 90, wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof.

92. The method of claim 81, wherein said one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof.

93. The method of claim 92, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof.

94. The method of claim 93, wherein said derivatives thereof comprise an epitope tag and full-length recombinant innate immunity pattern recognition receptors or recombinant soluble ectodomains thereof.

95. The method of claim 94, wherein said epitope tag comprises a N- or C-terminal 6×-histidine tag, green fluorescent protein (GFP), myc, hemagglutinin (HA), Fc fusion, biotin, or any combination thereof.

96. The method of claim 92, wherein said one or more antibodies, aptamers, single-chain variable fragments (scFv), or said single-chain antibodies comprise an epitope tag.

97. The method of claim 96, wherein said epitope tag comprises a N- or C-terminal 6×-histidine tag, green fluorescent protein (GFP), myc, hemagglutinin (HA), Fc fusion, biotin or any combination thereof.

98. The method of claim 81, wherein said one or more affinity reagents comprise a region to interact with said one or more microbial cell wall molecular motifs or said one or more non-microbial cell wall molecular motifs.

99. The method of claim 81, wherein said enriching comprises contacting said biological sample with a support comprising covalently immobilized affinity agents.

100. The method of claim 99, wherein said covalently immobilized affinity agents comprise a region that interacts with said one or more microbial cell wall molecular motifs or said one or more non-microbial cell wall molecular motifs.

101. The method of claim 99, wherein said supports comprise a magnetic bead, an agarose bead, non-magnetic latex, functionalized Sepharose, pH-sensitive polymers or any combinations thereof.

102. The method as in any of claim 81, wherein said enriching comprises:
(a) contacting said biological sample with said one or more affinity reagents to form capture reagent-molecular motif interaction complexes;
(b) contacting said capture reagent-molecular motif interaction complexes with a support; and
(c) separating said support from said biological sample to concentrate said capture reagent-molecular motif interaction complexes.

103. The method of claim 81, wherein said one or more affinity reagents comprise an epitope tag.

104. The method of claim 102, wherein said support comprises an epitope tag recognition surface.

105. The method of claim 104, wherein said epitope tag recognition surface comprises streptavidin, antibodies specific for 6×-histidine tag, green fluorescent protein (GFP), myc, hemagglutinin (HA), biotin, or any combination thereof.

106. The method of claim 104, wherein said epitope tag recognition surface comprises an anti-species antibody.

107. The method of claim 81, wherein said detecting comprises nucleic acid sequencing, polymerase chain reaction (PCR)-based, or hybridization-based analysis for nucleic acid analytes.

108. The method of claim 107, wherein said nucleic acid sequencing comprises whole genome sequencing, shotgun sequencing, next generations sequencing, targeted sequencing, RNA sequencing, methylation sequencing, or any combination thereof.

109. The method of claim 81, wherein said detecting comprises performing mass spectrometry analyses, liquid chromatography-mass spectrometry (LC-MS), or high-performance liquid chromatography (HPLC).

110. The method of claim 81, wherein said detecting comprises performing immunoassay analysis.

111. The method of claim 81, wherein said disease comprises cancer.

112. The method of claim 111, wherein said cancer comprises a stage I or stage II cancer.

113. The method of claim 111, wherein said cancer comprises bone, breast, lung, colon, brain, skin, ovary, pancreas, or any combination thereof type of cancer.

114. The method of claim 111, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

115. The method of claim 111, wherein said cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

116. The method of claim 81, wherein said identifying said treatment for said disease comprises predicting said treatment by a predictive model, wherein said predictive model is trained with one or more subjects' one or more molecular analytes obtained from one or more microbial extracellular vesicles and an associated treatment for said disease of said one more subjects.

117. The method of claim 116, wherein said predictive model is configured to receive said one or more molecular analytes of said subject an input, and output said treatment for said disease of said subject.

118. The method of claim 116, wherein said predictive model comprises a machine learning model, wherein said machine learning model comprises a regularized machine learning model, ensemble of machine learning models, or any combination thereof.

119. The method of claim 118, wherein said predictive model comprises a random forest, neural network, naïve bayes, support vector machines, learning regression, k-nearest neighbors, k-means, decision tree, logistic regression, gradient boosting, or any combination thereof.

120. The method of claim 116, wherein enriching said one or more microbial extracellular vesicles improves an accuracy of said predictive model by at least 1%, at least 5%, at least 10%, at least 15%, or at least 20% when predicting said treatment of said subject.

121. The method of claim 81, wherein said subject comprises a non-human mammal or a human subject.

122. The method of claim 81, wherein said treatment comprises a repurposed treatment, which may or may not have been originally approved for targeting cancer.

123. The method of claim 81, wherein said treatment comprises a small molecule, a biologic, a probiotic, a virus, a bacteriophage, an immunotherapy, a broad-spectrum antibiotic, or any combination thereof.

124. The method of claim 123, wherein said probiotic comprises an engineered bacterium strain or ensemble of engineered bacteria.

125. The method of claim 111, wherein said treatment comprises an adjuvant given in combination with a primary treatment against said cancer to improve an efficacy of said primary treatment.

126. The method of claim 111, wherein said treatment comprises adoptive cell transfer to target microbial antigens associated with said cancer or cancer microenvironment.

127. The method of claim 111, wherein said treatment comprises a cancer vaccine that exploits microbial antigens associated with said cancer or cancer microenvironment.

128. The method of claim 111, wherein said treatment comprises a monoclonal antibody against microbial antigens associated with said cancer or cancer microenvironment.

129. The method of claim 111, wherein said treatment comprises an antibody-drug conjugate designed to at least partially target microbial antigens associated with said cancer or cancer microenvironment.

130. The method of claim 111, wherein said treatment comprises a multi-valent antibody, antibody fragment, or antibody derivative thereof designed to at least partially target one or more microbial antigens associated with said cancer or cancer microenvironment.

131. The method of claim 81, wherein said treatment comprises a targeted antibiotic against a particular kind of microbe or class of functionally or biologically similar microbes.

132. The method of claim 111, wherein two or more of the following treatment types are combined such that at least one type exploits said cancer microbial presence or abundance to enhance overall therapeutic efficacy: small molecules, biologics, engineered host-derived cell types, probiotics, engineered bacteria, natural-but-selective viruses, engineered viruses, and bacteriophages.

133. A method of identifying a treatment for a disease of a subject, comprising:
(a) providing a biological sample of a subject comprising one or more microbial extracellular vesicles and one or more non-microbial extracellular vesicles;
(b) enriching said one or more microbial extracellular vesicles with one or more first affinity reagents and said one or more non-microbial extracellular vesicles with one or more second affinity reagents, thereby generating one or more enriched microbial extracellular vesicles and one or more enriched non-microbial extracellular vesicles;
(c) detecting a first abundance of one or more molecular analytes from said enriched one or more microbial extracellular vesicles and a second abundance of one or more molecular analytes from said enriched one or more non-microbial extracellular vesicles; and
(d) identifying said treatment for said disease of said subject from an association between a combination of said first abundance of one or more molecular analytes and said second abundance of one or more molecular analytes, and a model of disease treatments associated with a third abundance of one or more molecular analytes from one or more microbial extracellular vesicles and a fourth abundance of one or more molecular analytes from one or more non-microbial extracellular vesicles.

134. The method of claim 133, wherein detecting comprises quantifying said first abundance of one or more molecular analytes and said second abundance of one or more molecular analytes.

135. The method of claim 133, wherein said one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof.

136. The method of claim 133, wherein said one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof.

137. The method of claim 133, wherein said biological sample comprises a liquid biological sample, a tissue biological sample, or any combination thereof.

138. The method of claim 137, wherein said liquid biological sample comprises plasma, serum, whole blood, urine, cerebral spinal fluid, saliva, sweat, tears, lymphatic fluid, exhaled breath condensate, or any combination, any dilution, or processed fraction thereof.

139. The method of claim 138, wherein whole blood comprises white blood cells, plasma, red blood cells, platelets, or any combination thereof.

140. The method of claim 137, wherein the tissue biological sample comprises tissue homogenate.

141. The method of claim 133, wherein enriching comprises concentrating one or more microbial or one or more non-microbial cell wall molecular motifs.

142. The method of claim 141, wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, glycosylphosphatidylinositol (GPI)-anchored proteins, or any combination thereof.

143. The method of claim 133, wherein said one or more first affinity reagents or said one or more second affinity reagents comprise recombinant innate immunity pattern recognition receptors or one or more antibodies, wherein said one or more antibodies comprise polyclonal antibodies, monoclonal antibodies, recombinant antibodies, and aptamers, single-chain variable fragment (scFv), single-chain antibodies or any combination thereof.

144. The method of claim 143, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, or any derivatives thereof.

145. The method of claim 144, wherein said derivatives thereof comprise an epitope tag, full-length recombinant innate immunity pattern recognition receptors, or recombinant soluble ectodomains thereof.

146. The method of claim 143, wherein said one or more antibodies, said aptamers, said single-chain variable fragments (scFv), and said single-chain antibodies comprise an epitope tag.

147. The method of claim 146, wherein said epitope tag comprises a N- or C-terminal 6×-histidine tag, green fluorescent protein (GFP), myc, hemagglutinin (HA), Fc fusion, biotin, or any combination thereof.

148. The method of claim 133, wherein said one or more first affinity reagents and said one or more second affinity reagents comprise a region to interact with said one or more microbial cell wall molecular motifs or said one or more non-microbial cell wall molecular motifs.

149. The method of claim 133, wherein enriching comprises incubating said liquid biological sample with a support comprising covalently immobilized affinity agents.

150. The method of claim 149, wherein said covalently immobilized affinity agents comprise a region that interacts with said one or more microbial cell wall molecular motifs or said one or more non-microbial cell wall molecular motifs.

151. The method of claim 149, wherein said supports comprise a magnetic bead, an agarose bead, non-magnetic latex, functionalized Sepharose, pH-sensitive polymers or any combinations thereof.

152. The method of claim 133, wherein said one or more second affinity reagents comprise polyclonal, monoclonal, recombinant antibodies, aptamers, single-chain variable fragment (scFv), single-chain antibodies, or any combination thereof.

153. The method of claim 133, wherein said one or more first affinity reagents or said one or more second affinity reagents are specific to mammalian antigens CD9, CD63, CD81, glypican 1 (GPC1), Mart-1, TYRP2, Human epidermal growth factor receptors (HER) family members, EpCAM, or any combination thereof.

154. The method of claim 133, wherein detecting comprises performing nucleic acid sequencing, polymerase chain reaction (PCR), mass spectrometry analysis, liquid chromatography-mass spectrometry (LC-MS), high-performance liquid chromatography (HPLC), immunoassay analysis, or any combination thereof, with said one or more molecular analytes of said one or more microbial extracellular vesicles or said one or more molecular analytes from said one or more non-microbial extracellular vesicles.

155. The method of claim 154, wherein nucleic acid sequencing comprises whole genome sequencing, shotgun sequencing, next generations sequencing, targeted sequencing, RNA sequencing, methylation sequencing, or any combination thereof.

156. The method of claim 133, wherein said model comprises a predictive model, wherein said predictive model is trained with one or more subjects' said third abundance of one or more molecular analytes from said one or more microbial extracellular vesicles, said fourth abundance of one or more molecular analytes from said one or more non-microbial extracellular vesicles, and a corresponding treatment for said disease of said one or more subjects.

157. The method of claim 156, wherein said predictive model is configured to receive said first abundance of one or more molecular analytes and said second abundance of one or more molecular analytes as an input and output a prediction of said treatment for said disease of said subject.

158. The method of claim 133, wherein said disease comprises cancer.

159. The method of claim 158, wherein said cancer comprises a stage I or stage II cancer.

160. The method of claim 158, wherein said cancer comprises bone, breast, lung, colon, brain, skin, ovary, pancreas, or any combination thereof type of cancer.

161. The method of claim 158, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

162. The method of claim 158, wherein said cancer comprises one or more cancer types outside an intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

163. The method of claim 156, wherein said predictive model comprises a machine learning model.

164. The method of claim 163, wherein said machine learning model comprises one or more machine learning models, a regularized machine learning model, an ensemble of machine learning models, or any combination thereof.

165. The method of claim 156, wherein said predictive model comprises a random forest, neural network, naïve bayes, support vector machines, linear regression, k-nearest neighbors, k-means, decision tree, logistic regression, gradient boosting, or any combination thereof predictive model.

166. The method of claim 133, wherein said subject comprises a human or a non-human mammal.

167. The method of claim 156, wherein enriching said one or more microbial extracellular vesicles and said one or more non-microbial extracellular vesicles improves an accuracy of said predictive model by at least 1%, at least 5%, at least 10%, at least 15%, or at least 20% when predicting said treatment for said cancer of said subject.

168. The method of claim 156, wherein an area under a receiver operating characteristic curve of said predictive model predicting said treatment for said disease of said subject is increase by at least 1%, at least 2%, at least 4%, at least 5%, or at least 10%, when said combination of said first abundance of one or more molecular analytes and said second abundance of one or more molecular analytes are provided as said input to said predictive model.

169. The method of claim 133, wherein said treatment comprises a repurposed treatment, which may or may not have been originally approved for targeting cancer.

170. The method of claim 133, wherein said treatment comprises a small molecule, a biologic, a probiotic, a virus, a bacteriophage, an immunotherapy, a broad-spectrum antibiotic, or any combination thereof.

171. The method of claim 133, wherein said probiotic comprises an engineered bacterium strain or ensemble of engineered bacteria.

172. The method of claim 158, wherein said treatment comprises an adjuvant given in combination with a primary treatment against said cancer to improve an efficacy of said primary treatment.

173. The method of claim 158, wherein said treatment comprises adoptive cell transfer to target microbial antigens associated with said cancer or cancer microenvironment.

174. The method of claim 158, wherein said treatment comprises a cancer vaccine that exploits microbial antigens associated with said cancer or cancer microenvironment.

175. The method of claim 158, wherein said treatment comprises a monoclonal antibody against microbial antigens associated with said cancer or cancer microenvironment.

176. The method of claim 158, wherein said treatment comprises an antibody-drug conjugate designed to at least partially target microbial antigens associated with said cancer or cancer microenvironment.

177. The method of claim 158, wherein said treatment comprises a multi-valent antibody, antibody fragment, or antibody derivative thereof designed to at least partially target one or more microbial antigens associated with said cancer or cancer microenvironment.

178. The method of claim 133, wherein said treatment comprises a targeted antibiotic against a particular kind of microbe or class of functionally or biologically similar microbes.

179. The method of claim 158, wherein two or more of the following treatment types are combined such that at least one type exploits said cancer microbial presence or abundance to enhance overall therapeutic efficacy: small molecules, biologics, engineered host-derived cell types, probiotics, engineered bacteria, natural-but-selective viruses, engineered viruses, and bacteriophages.

180. A method of training a predictive model, comprising:
(a) providing a biological sample of one or more subjects comprising one or more microbial extracellular vesicles, and an associated health classification of said one or more subjects;
(b) enriching said one or more microbial extracellular vesicles with one or more affinity reagents, thereby generating one or more enriched microbial extracellular vesicles;
(c) detecting one or more molecular analytes from said enriched one or more microbial extracellular vesicles; and
(d) training said predictive model with said one or more molecular analytes and said associated health classification of said one or more subjects.

181. The method of claim 180, wherein said biological sample comprises non-microbial extracellular vesicles.

182. The method of claim 180, further comprising quantifying an abundance of said one or more molecular analytes.

183. The method of claim 180, wherein said one or more molecular analytes of said one or more microbial extracellular vesicles comprise vesicle-associated cell-free microbial DNA, cell-free microbial RNA, microbial DNA, microbial RNA, microbial proteins, microbial metabolites, microbial lipids, microbial glycans, or any combination thereof.

184. The method of claim 180, wherein said one or more molecular analytes of said non-microbial extracellular vesicles comprise vesicle-associated cell-free non-microbial DNA, cell-free non-microbial RNA, non-microbial DNA, non-microbial RNA, non-microbial proteins, non-microbial metabolites, non-microbial lipids, non-microbial glycans, or any combination thereof.

185. The method of claim 180, wherein said biological sample comprises a liquid biological sample, tissue biological sample, or any combination thereof.

186. The method of claim 185, wherein said liquid biological sample comprises plasma, serum, whole blood, urine, cerebral spinal fluid, saliva, sweat, tears, lymphatic fluid, exhaled breath condensate or any dilution, or processed fraction thereof.

187. The method of claim 186, wherein said whole blood comprises plasma, white blood cells, red blood cells, platelets, or any combination thereof.

188. The method of claim 185, wherein said tissue biological sample comprises tissue homogenate.

189. The method of claim 180, wherein said one or more affinity reagents are configured to couple to one or more microbial cell wall molecular motifs or one or more non-microbial cell wall molecular motifs.

190. The method of claim 189, wherein said one or more microbial cell wall molecular motifs or said one or more non-microbial cell wall molecular motifs comprise canonical cell wall components such as lipopolysaccharides (LPS), lipoproteins, lipopeptides, lipoteichoic acid (LTA), lipoarabinomannan, chitin, beta-glucans, zymosan, and glycosylphosphatidylinositol (GPI)-anchored proteins or any combinations thereof.

191. The method of claim 180, wherein said one or more affinity reagents comprise recombinant innate immunity pattern recognition receptors or one or more antibodies, wherein said one or more antibodies comprise polyclonal antibodies, monoclonal antibodies, recombinant antibodies, aptamers, single-chain variable fragment (scFv), single-chain antibodies, or any combination thereof.

192. The method of claim 191, wherein said recombinant innate immunity pattern recognition receptors comprise Toll-like receptor 1 (TLR1, CD281), Toll-like receptor 2 (TLR2, CD282), Toll-like receptor 4 (TLR4, CD284), Toll-like receptor 5 (TLR5), Toll-like receptor 6 (TLR6, CD286), Toll-like receptor 10 (TLR10, CD290), CD14, Lipopolysaccharide binding protein, Dectin-1, Dectin-2, Mannose Receptor (CD206), DC-SIGN, SIGNR1, Langerin, mannose binding lectin, ficolin-1, ficolin-2, ficolin-3, any combination thereof, or any derivatives thereof.

193. The method of claim 192, wherein said derivatives thereof comprise an epitope tag and full-length recombinant innate immunity pattern recognition receptors or recombinant soluble ectodomains thereof.

194. The method of claim 193, wherein said epitope tag comprises a N- or C-terminal 6×-histidine tag, green fluorescent protein (GFP), myc, hemagglutinin (HA), Fc fusion, biotin, or any combination thereof.

195. The method of claim 191, wherein said one or more antibodies, aptamers, single-chain variable fragments (scFv), or said single-chain antibodies comprise an epitope tag.

196. The method of claim 195, wherein said epitope tag comprises a N- or C-terminal 6×-histidine tag, green fluorescent protein (GFP), myc, hemagglutinin (HA), Fc fusion, biotin or any combination thereof.

197. The method of claim 180, wherein said one or more affinity reagents comprise a region to interact with said one or more microbial cell wall molecular motifs or said one or more non-microbial cell wall molecular motifs.

198. The method of claim 180, wherein said enriching comprises contacting said liquid biological sample with a support comprising covalently immobilized affinity agents.

199. The method of claim 198, wherein said covalently immobilized affinity agents comprise a region that interacts with said one or more microbial cell wall molecular motifs or said one or more non-microbial cell wall molecular motifs.

200. The method of claim 198, wherein said supports comprise a magnetic bead, an agarose bead, non-magnetic latex, functionalized Sepharose, pH-sensitive polymers or any combinations thereof.

201. The method of claim 180, wherein said enriching comprises:
(a) contacting said liquid biological sample with said one or more affinity reagents to form capture reagent-molecular motif interaction complexes;
(b) contacting said capture reagent-molecular motif interaction complexes with a support; and
(c) separating said support from said liquid biological sample to concentrate said capture reagent-molecular motif interaction complexes.

202. The method of claim 201, wherein said one or more affinity reagents comprise an epitope tag.

203. The method of claim 201, wherein said support comprises an epitope tag recognition surface.

204. The method of claim 203, wherein said epitope tag recognition surface comprises streptavidin, antibodies specific for 6×-histidine tag, green fluorescent protein (GFP), myc, hemagglutinin (HA), biotin, or any combination thereof.

205. The method of claim 204, wherein said epitope tag recognition surface comprises an anti-species antibody.

206. The method of claim 180, wherein said detecting comprises nucleic acid sequencing, polymerase chain reaction (PCR)-based, or hybridization-based analysis for nucleic acid analytes.

207. The method of claim 206, wherein said nucleic acid sequencing comprises whole genome sequencing, shotgun sequencing, next generations sequencing, targeted sequencing, RNA sequencing, methylation sequencing, or any combination thereof.

208. The method of claim 180, wherein said detecting comprises performing mass spectrometry analyses, liquid chromatography-mass spectrometry (LC-MS), or high-performance liquid chromatography (HPLC).

209. The method of claim 180, wherein said detecting comprises performing immunoassay analysis.

210. The method of claim 180, wherein said trained predictive model is configured to receive one or more molecular analytes of a subject as an input and output a predicted disease of said subject, a treatment for said disease of said subject, or any combination thereof.

211. The method of claim 210, wherein said disease comprises cancer.

212. The method of claim 211, wherein said cancer comprises a stage I or stage II cancer.

213. The method of claim 211, wherein said cancer comprises bone, breast, lung, colon, brain, skin, ovary, pancreas, or any combination thereof type of cancer.

214. The method of claim 211, wherein said cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, small cell lung carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

215. The method of claim 211, wherein said cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, lung small cell carcinoma, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

216. The method of claim 211, wherein said trained predictive model is configured to predict one or more cancers, one or more subtypes of cancer, the anatomical locations of one or more cancers, or any combination thereof of said subject.

217. The method of claim 211, wherein said trained predictive model is configured to predict a stage of said cancer, prognosis of said cancer, mutation status of said cancer, future immunotherapy response of said cancer, an optimal therapy to treat said cancer, or any combination thereof.

218. The method of claim 211, wherein said trained predictive model is configured to predict said cancer among one or more cancer types of said subject to identify a specific cancer type of said one or more cancer types.

219. The method of claim 180, wherein said trained predictive model comprises a machine learning model, wherein said machine learning model comprises a regularized machine learning model, ensemble of machine learning models, or any combination thereof.

220. The method of claim 219, wherein said trained predictive model comprises a random forest, neural network, naïve bayes, support vector machines, learning regression, k-nearest neighbors, k-means, decision tree, logistic regression, gradient boosting, or any combination thereof.

221. The method of claim 180, wherein enriching said microbial extracellular vesicles improves an accuracy of said trained predictive model by at least 1%, at least 5%, at least 10%, at least 15%, or at least 20% when predicting said cancer of said subject.

222. The method of claim 180, wherein said subject comprises a non-human mammal or a human subject.

223. The method of claim 180, wherein said health classification comprises cancerous, non-cancerous disease, or non-cancerous non-disease.

224. A computer-implemented method of training a predictive model, comprising:
(a) receiving one or more subjects' biological sample sequencing data and corresponding health classifications from a database, wherein said biological sample sequencing data comprises sequences of one or more analytes of one or more microbial extracellular vesicles; and
(b) training said predictive model with said biological sample sequencing data and said corresponding health classification of said one or more subjects.

225. A computer system configured to identify a disease of a subject, comprising:
(a) one or more processors; and
(b) a non-transient computer readable storage medium including software, wherein said software comprises executable instruction that, as a result of execution, cause said one or more processors of said computer system to:
(i) receive a biological sample of a subject comprising one or more microbial extracellular vesicles;
(ii) enrich said one or more microbial extracellular vesicles with one or more affinity reagents;
(iii) detect one or more molecular analytes from said one or more microbial extracellular vesicles; and
(iv) identify said disease of said subject based on said one or more molecular analytes obtained from said one or more microbial extracellular vesicles.
